SEQUENCE LISTING (29 sequences)

SEQ ID NO: 1 - DNA, 24 bp, synthetic construct
  source 1..24: mol_type = other DNA
tcagccatcaactctgctaccaac

SEQ ID NO: 2 - DNA, 26 bp, synthetic construct
  source 1..26: mol_type = other DNA
atctgaaacagccgtatatcatcttg

SEQ ID NO: 3 - DNA, 33 bp, synthetic construct
  source 1..33: mol_type = other DNA
ttctctgtcagctagccaatgatatcttcgagc

SEQ ID NO: 4 - DNA, 22 bp, synthetic construct
  source 1..22: mol_type = other DNA
gaggtcgagaagctgatattgg

SEQ ID NO: 5 - DNA, 22 bp, synthetic construct
  source 1..22: mol_type = other DNA
gtctcttcctatgcgggttatc

SEQ ID NO: 6 - DNA, 24 bp, synthetic construct
  source 1..24: mol_type = other DNA
acgtcactttgagggccaactgat

SEQ ID NO: 7 - DNA, 24 bp, synthetic construct
  source 1..24: mol_type = other DNA
aggtcttcaaggaactcagcaaga

SEQ ID NO: 8 - DNA, 21 bp, synthetic construct
  source 1..21: mol_type = other DNA
cggcaatcggtttgttgtaaa

SEQ ID NO: 9 - DNA, 20 bp, synthetic construct
  source 1..20: mol_type = other DNA
ccgtcagccattctctcggc

SEQ ID NO: 10 - DNA, 22 bp, synthetic construct
  source 1..22: mol_type = other DNA
gaggtcgagaagctgatattgg

SEQ ID NO: 11 - DNA, 22 bp, synthetic construct
  source 1..22: mol_type = other DNA
gtctcttcctatgcgggttatc

SEQ ID NO: 12 - DNA, 24 bp, synthetic construct
  source 1..24: mol_type = other DNA
acgtcactttgagggccaactgat

SEQ ID NO: 13 - DNA, 23 bp, synthetic construct
  source 1..23: mol_type = other DNA
cagccatcaactctgctaccaac

SEQ ID NO: 14 - DNA, 26 bp, synthetic construct
  source 1..26: mol_type = other DNA
atctgaaacagccgtatatcatcttg

SEQ ID NO: 15 - DNA, 33 bp, synthetic construct; identical to SEQ ID NO: 3
  source 1..33: mol_type = other DNA

SEQ ID NO: 16 - DNA, 20 bp, synthetic construct
  source 1..20: mol_type = other DNA
cccaggagtcccagtagtca

SEQ ID NO: 17 - DNA, 20 bp, synthetic construct
  source 1..20: mol_type = other DNA
cagttcctcgccttaggttg

SEQ ID NO: 18 - DNA, 23 bp, synthetic construct
  source 1..23: mol_type = other DNA
ttccccagatacacgacagaatc

SEQ ID NO: 19 - DNA, 17 bp, synthetic construct
  source 1..17: mol_type = other DNA
cggagcgcaggctacct

SEQ ID NO: 20 - DNA, 17 bp, synthetic construct
  source 1..17: mol_type = other DNA
cctacgtgttcgtcgac

SEQ ID NO: 21 - DNA, 25 bp, synthetic construct
  source 1..25: mol_type = other DNA
gttagggtataccgatgtgcgtgat

SEQ ID NO: 22 - DNA, 25 bp, synthetic construct
  source 1..25: mol_type = other DNA
gttagggtataccgatgtgcgtgat

SEQ ID NO: 23 - DNA, 27 bp, synthetic construct
  source 1..27: mol_type = other DNA
tccgaaacaactgtctgactggcaaaa

SEQ ID NO: 24 - DNA, 21 bp, synthetic construct
  source 1..21: mol_type = other DNA
agtggatgggcagcctatgta

SEQ ID NO: 25 - DNA, 20 bp, synthetic construct
  source 1..20: mol_type = other DNA
tcatatctgggtcccctgga

SEQ ID NO: 26 - DNA, 33 bp, synthetic construct
  source 1..33: mol_type = other DNA
aggtagaagaggttagggtgtttgatggcacag

SEQ ID NO: 27 - DNA, 21 bp, synthetic construct
  source 1..21: mol_type = other DNA
ctggtcatgtggatgctgtca

SEQ ID NO: 28 - DNA, 21 bp, synthetic construct
  source 1..21: mol_type = other DNA
gccagcaggctgttgatactg

SEQ ID NO: 29 - DNA, 17 bp, synthetic construct
  source 1..17: mol_type = other DNA
ccctttgtttggctgct

(19) United States
(12) Patent Application Publication
(10) Pub. No.: US 2025/0092080 A1
(43) Pub. Date: Mar. 20, 2025

(54) FLEX-NUCLEOSIDE ANALOGUES, NOVEL THERAPEUTICS AGAINST VIRUSES

(71) Applicant: UNIVERSITY OF MARYLAND, BALTIMORE COUNTY, Baltimore, MD (US)

(72) Inventor: Katherine L. RADTKE, Baltimore, MD (US)

(21) Appl. No.: 18/950,881

(22) Filed: Nov. 18, 2024

Related U.S. Application Data
(63) Continuation-in-part of application No. PCT/US2023/022464, filed on May 17, 2023.
(60) Provisional application No. 63/342,772, filed on May 17, 2022.
(60) Provisional application No. 63/351,843, filed on Jun. 14, 2022.
(60) Provisional application No. 63/377,360, filed on Sep. 28, 2022.

(51) Int. Cl. C07H 19/052; A61K 31/7064; A61K 31/7068; A61K 31/7072; A61P 31/20

(52) U.S. Cl. CPC C07H 19/052; A61K 31/7064; A61K 31/7068; A61K 31/7072; A61P 31/20

(57) ABSTRACT

Compounds, methods, and compositions for treating or preventing viral infections using flexible nucleosides analogs. The fleximer nucleoside analogues having increased flexibility and ability to alter their conformation structures to provide increased antiviral activity potential with the result of inhibiting at least one of flaviviruses, herpesviruses, polyomaviruses, alphaviruses, enteroviruses, filoviruses matonavirus, phenuivirus and/or coronaviruses.

### CROSS REFERENCE TO RELATED APPLICATIONS

This application is a continuation-in-part of, and claims priority to, International Patent Application No. PCT/US2023/022464 filed on May 17, 2023, which claims priority to each of U.S. Provisional Patent Application No. 63/342,772 filed on May 17, 2022 in the name of Katherine L. Radtke and entitled “Design, synthesis and methods of use of fleximer nucleoside analogues having anti-coronavirus activity against SARS-CoV-2 and COVID-19 and mutants thereof,” U.S. Provisional Patent Application No. 63/351,843 filed on Jun. 14, 2022 in the name of Katherine L. Radtke and entitled “Design, synthesis and methods of use of fleximer nucleoside analogues having anti-virus activity,” and U.S. Provisional Patent Application No. 63/377,360 filed on Sep. 28, 2022 in the name of Katherine L. Radtke and entitled “Flex-nucleoside analogues, novel therapeutics against coronaviruses, herpesviruses, alphaviruses, polyomaviruses, enteroviruses, filoviruses, matonaviruses, phenuiviruses, and flaviviruses,” which are all hereby incorporated by reference herein in their entirety.

### SEQUENCE LISTING

The text of the computer readable sequence listing filed herewith, titled “UMBC_40380_504_SequenceListing.xml,” created Nov. 18, 2024, having a file size of 26,750 bytes, is hereby incorporated by reference in its entirety.

### FIELD

The present invention is directed to nucleoside analogue compounds, compositions comprising same, and methods for treating or preventing any one of coronaviruses, herpesviruses, alphaviruses, enteroviruses, polyomaviruses, filoviruses, matonaviruses, phenuiviruses and/or flaviviruses using said nucleosides analogues. Specifically, the present invention provides for the design and synthesis of fleximer nucleoside analogues having increased flexibility and ability to alter their conformation to provide increased antiviral activity potential that can inhibit several viruses.

### BACKGROUND

Viruses are small infectious agents that can only multiply within the cells of animals, plants, and bacteria. The structures of viruses are simple compared to living cells and contain a small haploid DNA or

RNA genome and a protein or glycoprotein coat called a capsid. In addition, some viruses called enveloped viruses are surrounded by a lipid membrane.

A number of viruses appear on the United States National Institutes of Allergy and Infectious Disease (NIAID) list of Emerging Diseases/Pathogens list, which include Coronaviruses (e.g., SARS-1, SARS-2, Middle East respiratory syndrome (MERS), COVID-19, and mutants thereof), Flaviviruses (e.g., Dengue, Zika, yellow fever, tickborne encephalitis, HCV, and West Nile), Herpesviruses (e.g., herpes simplex virus 1, herpes simplex virus 2, varicella-zoster virus, Epstein-Barr virus, cytomegalovirus, Human herpesvirus-6, Human herpesvirus-7, and Kaposi's sarcoma herpes virus), alphavirus (e.g., eastern equine encephalomyelitis (EEE), Venezuelan equine encephalomyelitis (VEE) and western equine encephalomyelitis (WEE)), enteroviruses (e.g., echovirus and coxsackievirus), Filoviruses (e.g., Ebola virus, Sudan virus, and Marburg virus), matonaviruses (e.g., Rubella, Rustrela, and Ruhugu), and phenuiviruses (e.g., Rift Valley Fever virus) to name a few.

Filoviruses are enveloped viruses with a genome consisting of one linear single-stranded RNA segment of negative polarity. The viral genome encodes 7 proteins. Nucleoprotein (NP), virion protein 35 kDa (VP35) and virion protein 30 kDa (VP30) are associated with the viral ribonucleoprotein complex. Members of the filovirus genus include Zaire Ebola virus, Sudan Ebola virus, Reston Ebola virus, Cote d′Ivoire Ebola virus and Marburg virus. Ebola and Marburg viruses can cause severe hemorrhagic fever and have a high mortality rate. Ebola virus (Zaire and Sudan species) was first described in 1976 after outbreaks of a febrile, rapidly fatal hemorrhagic illness were reported along the Ebola River in Zaire (now the Democratic Republic of the Congo) and Sudan. The natural host for Ebola viruses is still unknown but is widely speculated to be bats. Marburg virus, named after the German town where it was first reported in 1967, is primarily found in equatorial Africa. The host range of Marburg virus includes non-human and human primates.

Viruses in the genus flavivirus are known to cause viral hemorrhagic fevers (VHFs). Flaviviruses are enveloped viruses with a genome consisting of one linear single-stranded RNA segment of positive polarity. The polyprotein is co- and post-transcriptionally cleaved by cell signal peptidase and the viral protease to generate individual viral proteins. Viral structural proteins include capsid (C), precursor to M (prM), minor envelope (M) and major envelope (E). Members of the flavivirus genus include yellow fever virus, Apoi virus, Aroa virus, Bagaza virus, Banzi virus, Bouboui virus, Bukalasa bat virus, Cacipacore virus, Carey Island virus, Cowbone Ridge virus, Dakar bat virus, dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Phenh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, tick-borne encephalitis virus, Tyuleniy virus, Uganda S virus, Usutu virus, Wesselsbron virus, West Nile virus, Yaounde virus, Yokose virus, Zika virus, cell fusing agent virus and Tamana bat virus.

Coronaviruses are enveloped viruses, having a capsid exhibiting a helical symmetry. They have a single-stranded positive sense RNA genome and are capable of infecting cells from birds and mammals. The viruses, which are members of this very wide family, are known to be causative agents for the common cold (for example, human coronaviruses (HCoV) 229E and OC43), bronchiolitis (for example NL63 virus) or even some forms of pneumoniae, e.g., as those observed during the SARS (such as the Severe Acute Respiratory Syndrome Coronavirus, SARS-CoV) epidemic. In January 2020 the World Health Organization reported infection from a new novel coronavirus SARS-CoV-2 (COVID-19 disease) which has shown a higher mortality rate than MERS-CoV or SARS-CoV and exhibits an ongoing risk of human-to-human transmission.

There are relatively few prophylactic or therapeutic agents for treatment of viral diseases caused by Coronaviruses, Alphaviruses, Enteroviruses, Herpesviruses. Flaviviruses, Matonaviridae, Phenuiviridae and/or Filoviruses. The need for new and more effective antiviral therapeutics, particularly those targeting emerging and reemerging infectious diseases and pathogens continues to increase. Thus, in light of the above discussion, there is a need for discovering and providing new and more efficient antiviral drugs.

### SUMMARY

The present invention provides for flexible and modified nucleoside analogues that allow access to more potential binding sites with the ability to retain their potency against viral diseases caused by a virus including, but not limited to, Coronaviruses, Polyomaviruses, Herpesviruses. Alphaviruses, Enteroviruses, Flaviviruses, Matonaviruses, Phenuiviruses and Filoviruses, since the flexible and modified nucleoside analogues of the present invention can “wiggle and jiggle” in the binding site. These findings are causing a paradigm shift in drug design having antiviral activity.

In one aspect, flexible nucleoside analogues are disclosed, selected from at least one of: (i) at least one compound comprising a Flex-Acyclovir scaffold:

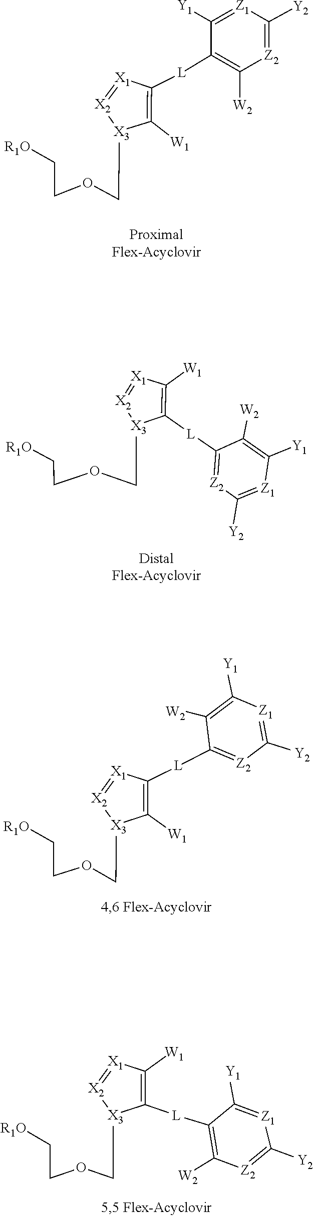

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, monophosphate, diphosphate, triphosphate, various prodrugs (e.g., esters, McGuigan ProTides, lipid phosphates, lipid esters, HepDirect, amino acid prodrugs) or —C(O)-alkyl;
   (ii) at least one compound comprising a Flex-tenofovir scaffold:

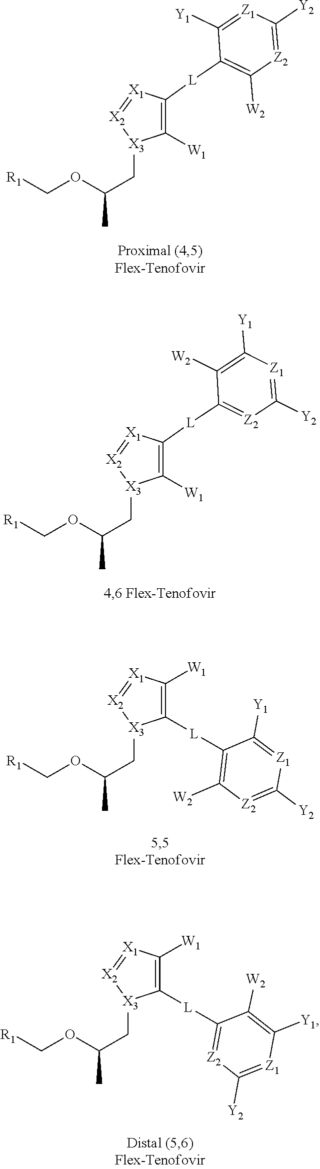

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, phosphonate, monophosphate phosphonate, and diphosphate phosphonate, various prodrugs (e.g., esters, McGuigan ProTides, diisoproxyl fumarate, lipid phosphates, lipid esters, HepDirect, amino acid prodrugs); and

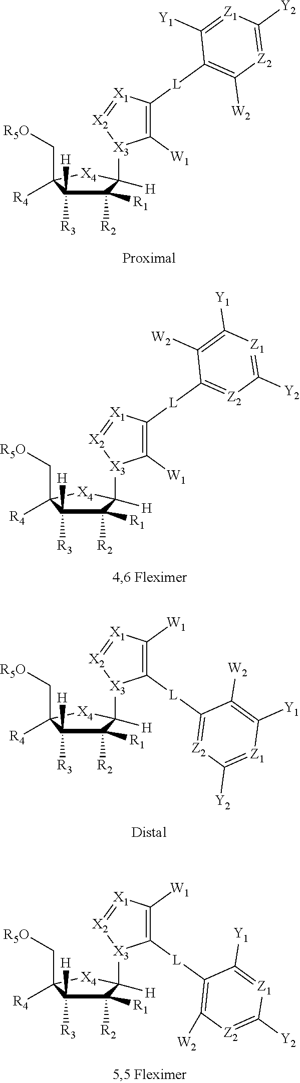

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof.

In another aspect, a pharmaceutical composition comprising at least one of the flexible nucleoside analogue and at least one pharmaceutically acceptable carrier is disclosed, wherein the at least one flexible nucleoside analogue is selected from at least one of:

(i) at least one compound comprising a Flex-Acyclovir scaffold:

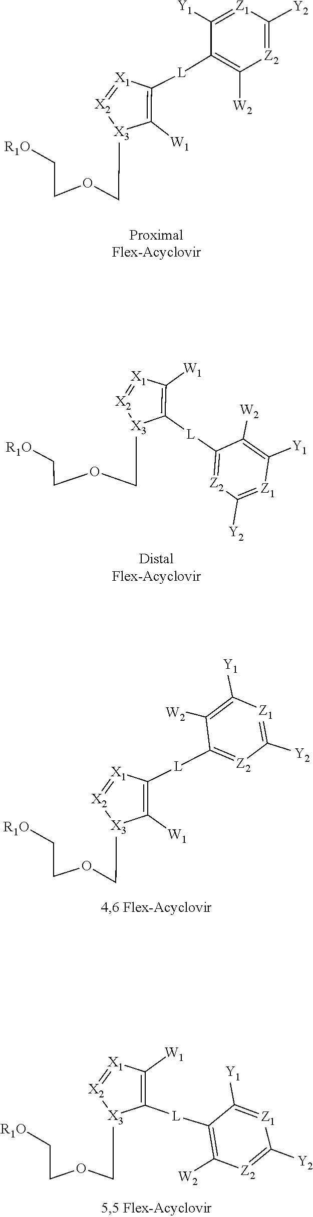

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, monophosphate, diphosphate, triphosphate, various prodrugs (e.g., esters, McGuigan ProTides, lipid phosphates, lipid esters, HepDirect, amino acid prodrugs) or —C(O)-alkyl;
   (ii) at least one compound comprising a Flex-tenofovir scaffold:

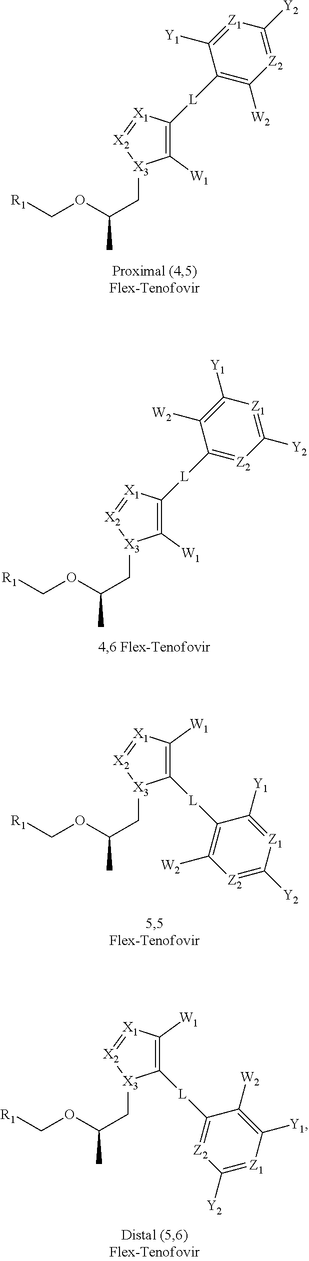

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, phosphonate, monophosphate phosphonate, and diphosphate phosphonate, various prodrugs (e.g., esters, McGuigan ProTides, diisoproxyl fumarate, lipid phosphates, lipid esters, HepDirect, amino acid prodrugs); and

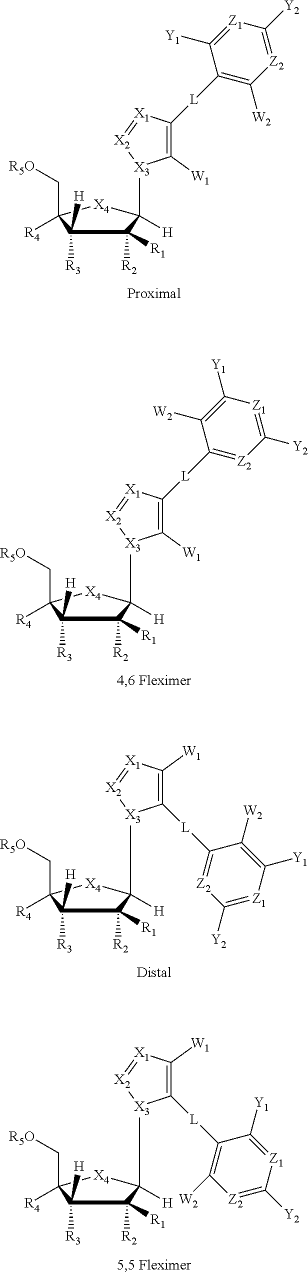

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs),
   or a pharmaceutically acceptable salt, isomer, hydrate, prodrug or solvate thereof.

In yet another aspect, a method for treating and/or preventing a viral infection in a subject is disclosed, wherein the viral infection is caused by at least one of a coronavirus, a herpesvirus, an alphavirus, a polyomavirus, an enterovirus, a filovirus, a matonavirus, a phenuivirus, and/or a flavivirus, comprising administration, to the subject, of a therapeutically effective amount of at least one fleximer nucleoside analogue, or a pharmaceutical composition comprising same, wherein the at least one fleximer nucleoside analogue is selected from at least one of:

(i) at least one compound comprising a Flex-Acyclovir scaffold:

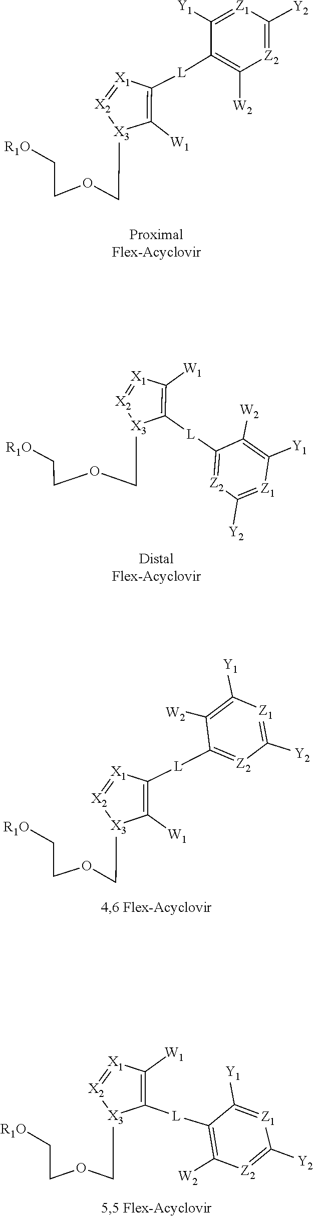

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, monophosphate, diphosphate, triphosphate, various prodrugs (e.g., esters, McGuigan ProTides, lipid phosphates, lipid esters, HepDirect, amino acid prodrugs) or —C(O)-alkyl;
   (ii) at least one compound comprising a Flex-tenofovir scaffold:

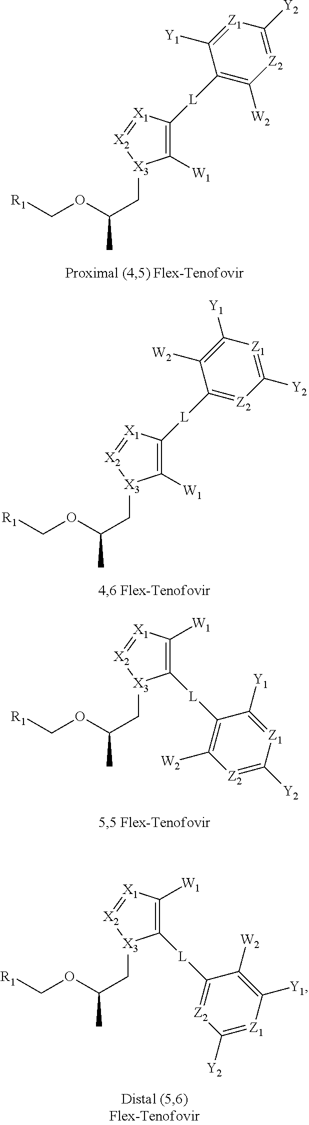

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, phosphonate, monophosphate phosphonate, and diphosphate phosphonate, various prodrugs (e.g., esters, McGuigan ProTides, diisoproxyl fumarate, lipid phosphates, lipid esters, HepDirect, amino acid prodrugs); and
   (iii) at least one compound comprising D-nucleoside fleximer:

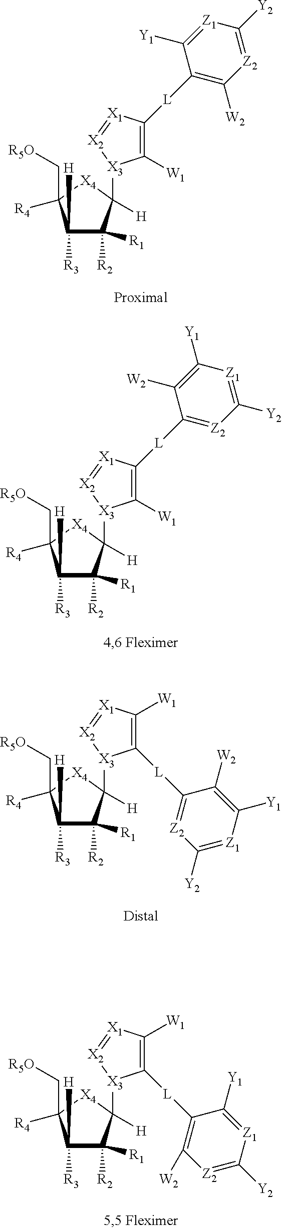

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs),
   or a pharmaceutically acceptable salt, isomer, hydrate, prodrug or solvate thereof.

Other aspects, features and embodiments of the invention will be more fully apparent from the ensuing disclosure and appended claims.

### BRIEF DESCRIPTION OF THE FIGURES

FIG. 1. An embodiment of a process to synthesize CHK-03.

FIG. 2. An embodiment of a process to synthesize CHK-05.

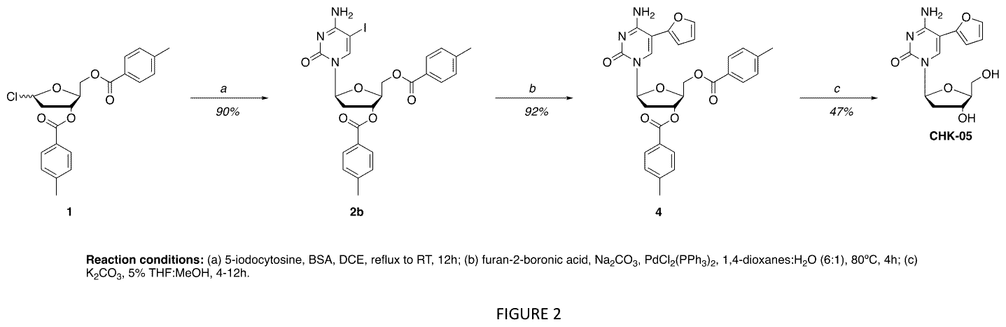

FIG. 3. An embodiment of a process to synthesize JT-001, JT-004, JT-005, and JT-006.

FIG. 4. An embodiment of a process to synthesize CDW3-002.

### DETAILED DESCRIPTION

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As defined herein, “alkyl” group corresponds to a C₁-C₆ straight or branched-chain group, having the general formula of CₙH₂ₙ₊₁ including, but not limited to, methyl, ethyl, n-propyl, isopropyl, n-butyl, sec-butyl, isobutyl, t-butyl, n-pentyl, 1-methylbutyl, 2-methylbutyl, 3-methylbutyl, 1-ethylpropyl, 1,1-dimethylpropyl, 1,2-dimethylpropyl, 2,2-dimethylpropyl, n-hexyl, 4-methylpentyl, 3-methylpentyl, 2-methylpentyl, 1-methylpentyl, 1,3-dimethylbutyl, 1,2-dimethylbutyl, 3,3-dimethylbutyl, 1-methyl-2,2-dimethylpropyl, 1-ethylbutyl, 1-ethyl-2-methylpropyl, and 2-ethylbutyl. In addition, an alkyl group includes a C₃-C₆ cycloalkyl group having the general formula of CₙH₂ₙ₋1 including, but not limited to, cyclopropyl, cyclobutyl, cyclopentyl, cyclopentenyl, and cyclohexyl.

As defined herein, a “halide” corresponds to fluoride, chloride, bromide or iodide.

As defined herein, an “aryl” group corresponds to a functional group derived from an aromatic ring (e.g., an aromatic hydrocarbon ring) by the removal of one hydrogen atom.

As defined herein, a “heteroatom” includes nitrogen, oxygen, sulfur, and silicon. In some embodiments, the heteroatom is nitrogen.

“Subject” as used herein refers to any vertebrate such as mammals, birds, reptiles, amphibians and fish including, but not limited to, a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, monkey, chimpanzee, and humans. In some embodiments, the subject is a human.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

As defined herein, “lipid phosphates” include, but are not limited to,

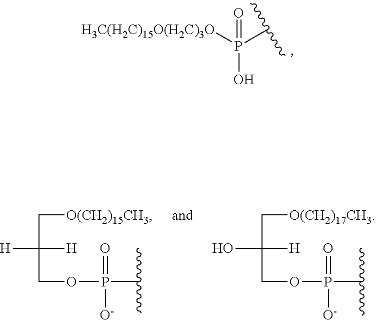

As defined herein, “McGuigan ProTides” correspond to prodrugs of monophosphate nucleotide analogs, including phosphoramidate prodrugs (known as “ProTide” prodrugs). These nucleotide ProTide prodrugs were developed by Prof. McGu example as described in PCT Applications WO90/05736; WO90/10012; WO9629336; WO2000/0 O2001/083501; WO2001085749; WO2003/061670; and WO2005/012327, which are incorporated herein by reference in their entirety. The McGuigan ProTides have been successfully applied to a vast number of nucleoside analogues with antiviral and anticancer activity ProTides consist of a 5′-nucleoside monophosphate in which the two hydroxyl groups are masked with an amino acid ester and an aryloxy component which once in the cell is enzymatically metabolized to deliver free 5′-monophosphate, which is further transformed to the active 5′-triphosphate form of the nucleoside analogue. In some embodiments, the McGuigan Protide has the structure:

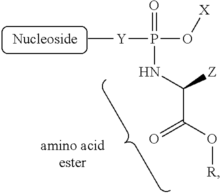

wherein
X is a substituted or unsubstituted aryl, naphthalene or biphenyl, wherein the substitutions include, but are not limited to, OH, NH₂, F, alkyl, O-alkyl, and/or NH-alkyl;
Z is the side chain of the amino acid including, but not limited to, methyl (i.e., the amino acid is alanine), and isopropyl (i.e., the amino acid is valine);
Y is O or CH₂; and
R is an alkyl or aryl group including, but not limited to, methyl, isopropyl, isobutyl, sec-butyl, or ethyl butyl.

### Flexible Nucleoside Analogues

Unique nucleoside analogues have been termed “fleximers” and were designed to explore how nucleobase flexibility affects the recognition, binding, and activity of nucleoside(tide) analogues. The fleximers possess a purine base scaffold in which the imidazole and pyrimidine moieties are attached by a single carbon-carbon bond, rather than being ‘fused’ as is typical for the purines. These analogues are designed to retain all of the requisite purine hydrogen bonding patterns while allowing the nucleobase to explore alternative binding modes. In some embodiments, fleximers allow access to more potential binding sites with the ability to retain their potency against resistant cancers and viral strains since they can “wiggle and jiggle” in the binding site. These findings are causing a paradigm shift in drug design having anticancer and antiviral activity.

The fleximers described herein may include several types of modifications: that being parent prox- and distal fleximers connected at 4,5 or 5,6, fleximers connected at 4,6 and 5,5, as well as fleximers with a linker positioned between the two heterocyclic base pieces, substituent(s) on the inside of the two heterocyclic pieces to hold the rotation of the flex-base in a particular conformation referred to as T3 analogues (“tune the twist”).

In a first aspect, the present invention relates to fleximer nucleoside analogues. In some embodiments of the first aspect, a series of flexible nucleoside analogues are described, wherein the flexible nucleoside analogues are selected from at least one of:

(i) at least one compound comprising a Flex-Acyclovir scaffold:

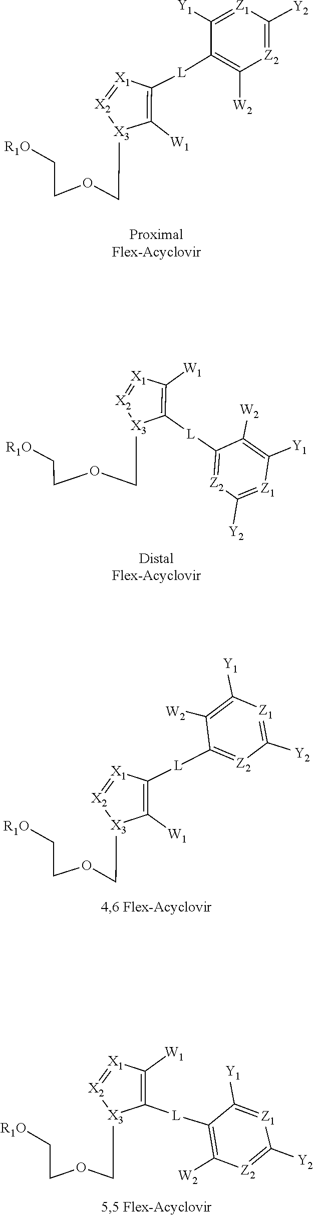

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, monophosphate, diphosphate, triphosphate, various prodrugs (e.g., esters, McGuigan ProTides, lipid phosphates, lipid esters, HepDirect, amino acid prodrugs) or —C(O)-alkyl;
   (ii) at least one compound comprising a Flex-tenofovir scaffold:

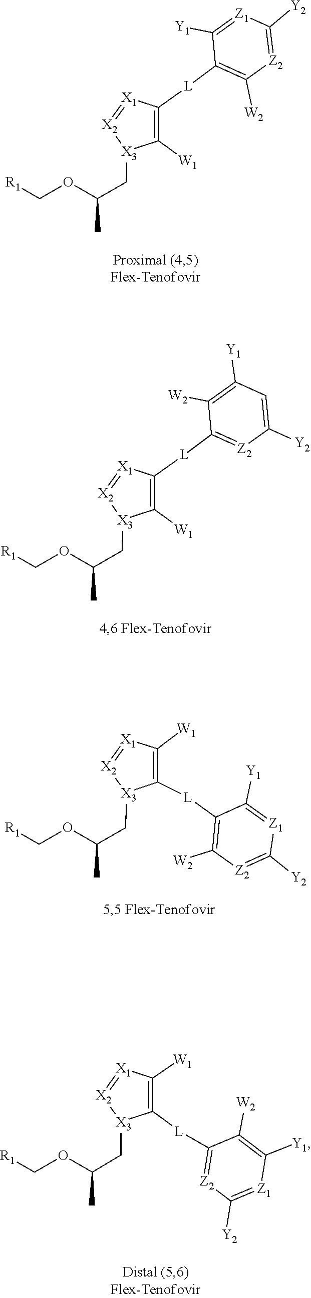

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, phosphonate, monophosphate phosphonate, and diphosphate phosphonate, various prodrugs (e.g., esters, McGuigan ProTides, diisoproxyl fumarate, lipid phosphates, lipid esters, HepDirect, amino acid prodrugs);
   (iii) at least one compound comprising a Flex-Cidofovir scaffold:

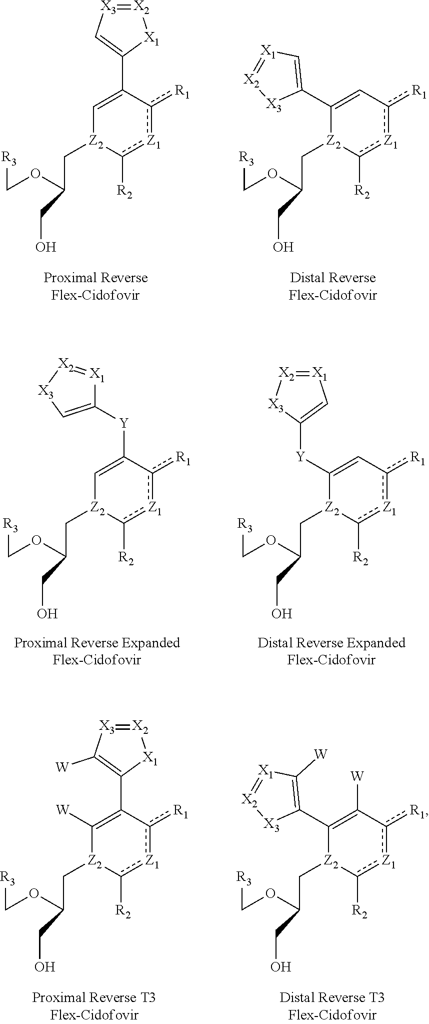

wherein
X₁ is C or N;
X₂ and X₃ are each independently selected from C, O, N, or S;
Y is CH₂, O, NH, S, vinyl, or ethynyl;
W is H, NH₂, OH, CH₃, O-alkyl, or NH-alkyl;
Z₁ and Z₂ are each independently selected from C or a heteroatom;
R₁ and R₂ are each independently selected from H, OH, F, NH₂, NH-alkyl, OMe, or NH—OH; and
R₃ is H, phosphonate, monophosphate phosphonate, and diphosphate phosphonate, various prodrugs (e.g., esters, McGuigan ProTides, lipid phosphates, lipid esters, HepDirect, amino acid prodrugs);
   (iv) at least one compound comprising a Flex-Sofosbuvir/Molnupiravir scaffold:

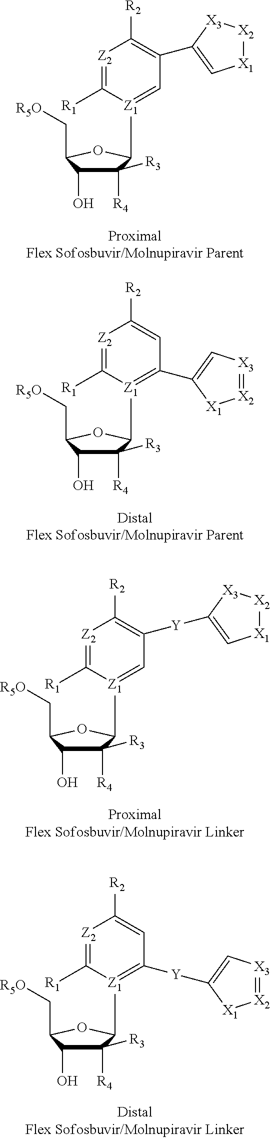

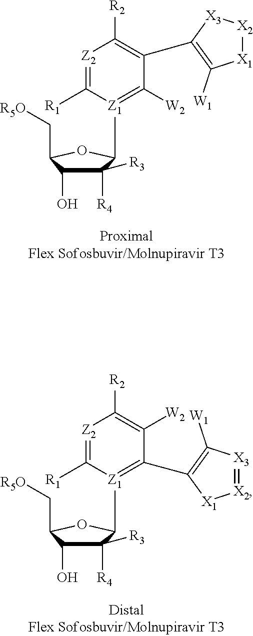

wherein
R₁ is H, NH₂, OH, O-alkyl, NH-alkyl, or F;
R₂ is H, NH₂, OH, NHOH, O-alkyl, or NH-alkyl;
R₃ is H, CH₃, or ethynyl;
R₄ is H, F, Cl, or OH;
R₅ is H, monophosphate, diphosphate, triphosphate, various prodrugs (e.g., esters, McGuigan ProTides, lipid phosphates, lipid esters, HepDirect, amino acid prodrugs);
Z₁ and Z₂ are each independently selected from C or N;
X₁, X₂, and X₃ are each independently selected from C, O, N, or S;
Y is NH, CH₂, O, ethynyl, or ethenyl;
W₁ is F, CH₃, or ethynyl; and
W₂ is OH, NH₂, O-alkyl, NH-alkyl, or F;
   (v) at least one compound comprising a Flex-BCNA scaffold:

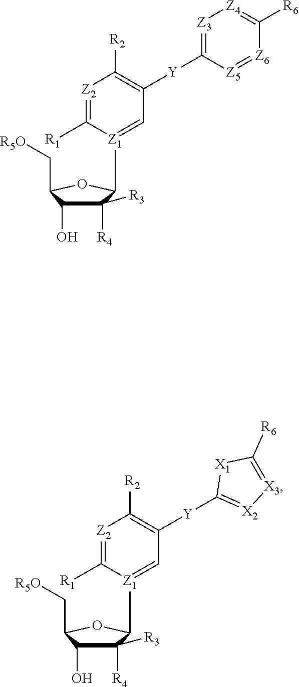

wherein
R₁ is H, NH₂, OH, O-alkyl, NH-alkyl, or F;
R₂ is H, NH₂, OH, NHOH, O-alkyl, or NH-alkyl;
R₃ is H, CH₃, ethynyl;
R₄ is H, F, Cl, or OH;
R₅ is H, monophosphate, diphosphate, triphosphate, various prodrugs (e.g., esters, McGuigan ProTides, lipid phosphates, lipid esters, HepDirect, amino acid prodrugs);
R₆ is NH₂, OH, alkyl, halide, NO₂, O-alkyl, NH-alkyl, COOH, CONH₂, CNHNH₂;
Z₁, Z₂, Z₃, Z₄, Z₅, and Z₆ are each independently selected from C or N;
X₁, X₂ and X₃ are each independently selected from C, O, N, or S; and
Y is NH(CH₂)ₙ, O(CH₂)ₙ, ethynyl, or ethenyl;
   (vi) at least one compound comprising D-nucleoside fleximer:

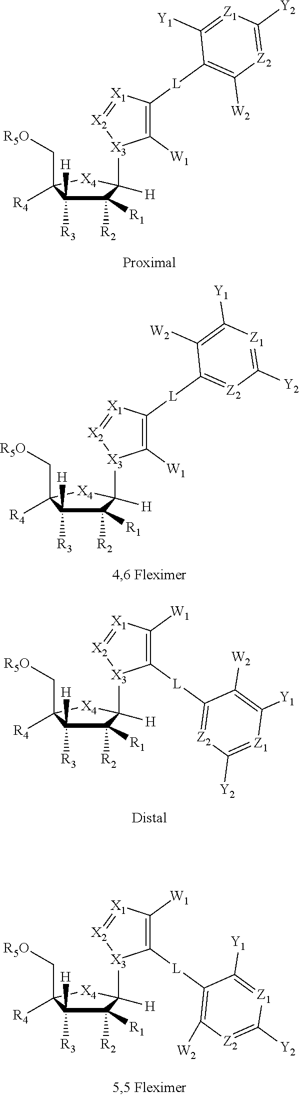

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs); and
   (vii) at least one reverse fleximer:

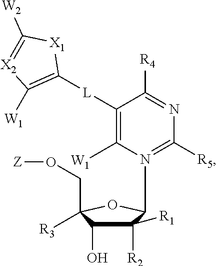

wherein
X₁ and X₂ are each independently selected from C or a heteroatom;
W₁ is H, NH₂, OH, O-alkyl, CF₃, NH-alkyl, F, or CH₃;
W₂ is H, NH₂, OH, alkyl, O-alkyl, CF₃, NH-alkyl, F, or NO₂;
R₁ is H, CH₃, or alkynyl;
R₂ is H, F, or OH;
R₃ is H;
R₄ is ═O, NH₂, NH-alkyl, or O-alkyl;
R₅ is ═O;
L is null, NH, CH₂, O, vinyl, ethynyl; and
Z is H, monophosphate, diphosphate, triphosphate, various prodrugs (e.g., esters, McGuigan ProTides, lipid phosphates, lipid esters, HepDirect, amino acid prodrugs),
   or a pharmaceutically acceptable salt, isomer, hydrate, prodrug or solvate thereof.

In some embodiments of the first aspect, the flexible nucleoside analogue comprises at least one of:

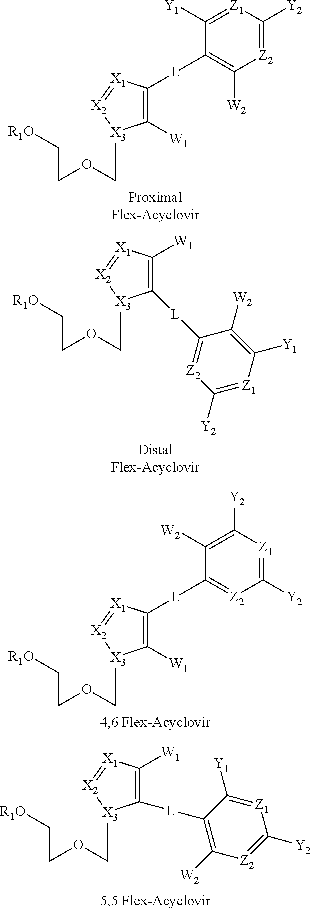

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, monophosphate, diphosphate, triphosphate, various prodrugs (e.g., esters, McGuigan ProTides, lipid phosphates, lipid esters, HepDirect, amino acid prodrugs),
   or a pharmaceutically acceptable salt, isomer, hydrate, prodrug or solvate thereof.
   In some embodiments, X₁=X₃=N and X₂ is C. In some embodiments, Z₁=Z₂=N. In some embodiments, W₁=W₂=H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, and L is null.

In some embodiments of the first aspect, the flexible nucleoside analogue comprises at least one of:

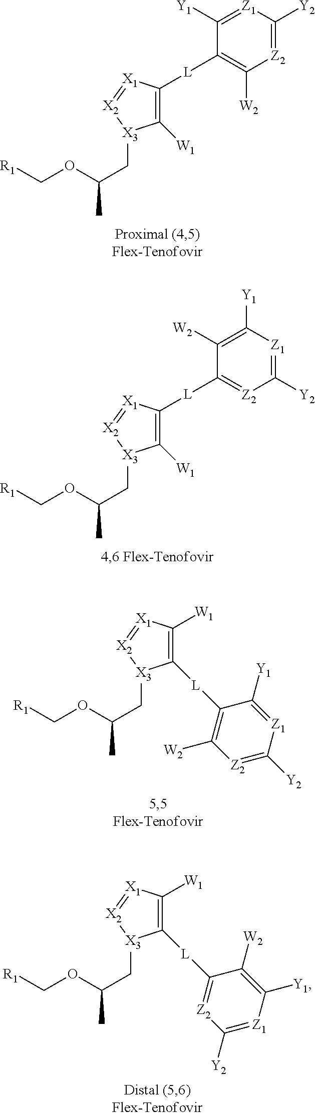

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, phosphonate, monophosphate phosphonate, and diphosphate phosphonate, various prodrugs (e.g., esters, McGuigan ProTides, lipid phosphates, lipid esters, HepDirect, amino acid prodrugs),
   or a pharmaceutically acceptable salt, isomer, hydrate, prodrug or solvate thereof.
   In some embodiments, X₁=X₃=N and X₂ is C. In some embodiments, Z₁=Z₂=N. In some embodiments, W₁=W₂=H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, and L is null.

In some embodiments of the first aspect, the flexible nucleoside analogue comprises at least one of:

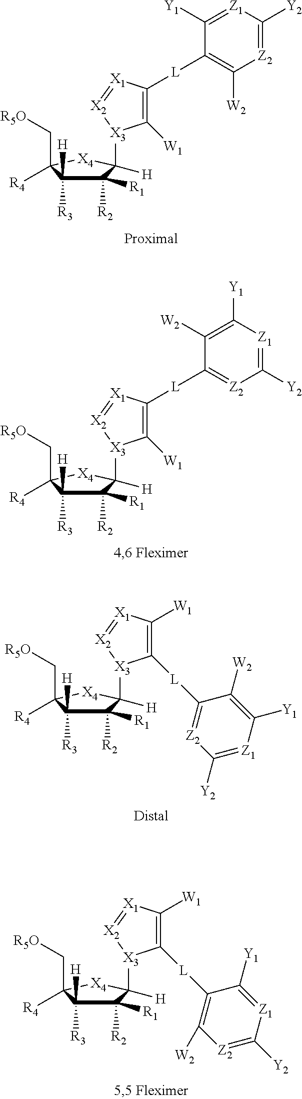

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs),
   or a pharmaceutically acceptable salt, isomer, hydrate, prodrug or solvate thereof.
   In some embodiments, X₁=X₃=N and X₂ is C. In some embodiments, Z₁=Z₂=N. In some embodiments, W₁=W₂=H. In some embodiments, R₁ is CH₃, R₂ is F, R₃ is OH and/or R₄ is H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and R₁ is CH₃, R₂ is F, R₃ is OH and/or R₄ is H. In some embodiments, X₁=X₃=N, X₂ is C, and L is null. In some embodiments, R₁ is CH₃, R₂ is F, R₃ is OH and/or R₄ is H and Z₁=Z₂=N. In some embodiments, R₁ is CH₃, R₂ is F, R₃ is OH and/or R₄ is H and W₁=W₂=H. In some embodiments, R₁ is CH₃, R₂ is F, R₃ is OH and/or R₄ is H and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, R₁ is CH₃, R₂ is F, R₃ is OH and/or R₄ is H, and L is null.

In some embodiments of the first aspect, the flexible nucleoside analogue comprises at least one of:

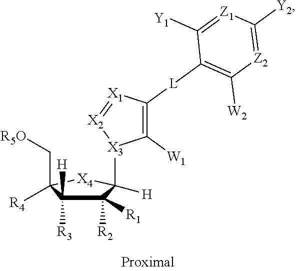

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs),
   or a pharmaceutically acceptable salt, isomer, hydrate, prodrug or solvate thereof.
   In some embodiments, X₁=X₃=N and X₂ is C. In some embodiments, Z₁=Z₂=N. In some embodiments, W₁=W₂=H. In some embodiments, R₁ is CH₃, R₂ is F, R₃ is OH and/or R₄ is H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and R₁ is CH₃, R₂ is F, R₃ is OH and/or R₄ is H. In some embodiments, X₁=X₃=N, X₂ is C, and L is null. In some embodiments, R₁ is CH₃, R₂ is F, R₃ is OH and/or R₄ is H and Z₁=Z₂=N. In some embodiments, R₁ is CH₃, R₂ is F, R₃ is OH and/or R₄ is H and W₁=W₂=H. In some embodiments, R₁ is CH₃, R₂ is F, R₃ is OH and/or R₄ is H and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, R₁ is CH₃, R₂ is F, R₃ is OH and/or R₄ is H, and L is null. In some embodiments, Y₁ is selected from the group consisting of —NH—CH₃, —O—CH₃, —NH-cyclopropyl, NH₂, and ═O. In some embodiments, Y₂ is selected from the group consisting of —NH₂, —O—CH₃, F, CH₃, H, —NH—CH₃, and ═O.

In some embodiments of the first aspect, the flexible nucleoside analogue comprises at least one of:

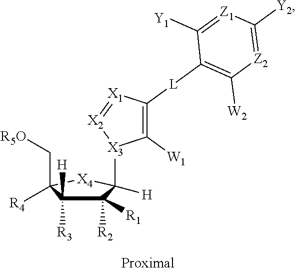

L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H,

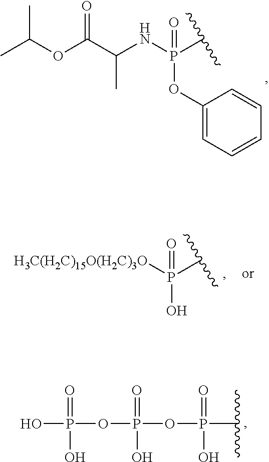

or a pharmaceutically acceptable salt, isomer, hydrate, prodrug or solvate thereof.
In some embodiments, X₁=X₃=N and X₂ is C. In some embodiments, Z₁=Z₂=N. In some embodiments, W₁=W₂=H. In some embodiments, R₁ is CH₃, R₂ is F, R₃ is OH and/or R₄ is H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and R₁ is CH₃, R₂ is F, R₃ is OH and/or R₄ is H. In some embodiments, X₁=X₃=N, X₂ is C, and L is null. In some embodiments, R₁ is CH₃, R₂ is F, R₃ is OH and/or R₄ is H and Z₁=Z₂=N. In some embodiments, R₁ is CH₃, R₂ is F, R₃ is OH and/or R₄ is H and W₁=W₂=H. In some embodiments, R₁ is CH₃, R₂ is F, R₃ is OH and/or R₄ is H and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, R₁ is CH₃, R₂ is F, R₃ is OH and/or R₄ is H, and L is null.

In some embodiments of the first aspect, the flexible nucleoside analogue comprises at least one of:

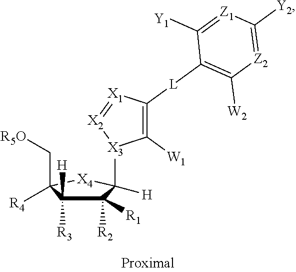

wherein
L is null;
X₁ and X₃ are each N;
X₂ is C;
X₄ is O;
W₁ and W₂ are each H;
Y₁ and Y₂ are each independently selected from H, ═O, OH, S, F, NH₂, NH-alkyl, O-alkyl, alkyl, or NH—OH;
Z₁ and Z₂ are N;
R₁ is alkyl;
R₂ is F;
R₃ is OH;
R₄ is H; and
R₅ is H, monophosphate, diphosphate, triphosphate, various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof.

In some embodiments of the first aspect, the flexible nucleoside analogue comprises at least one of:

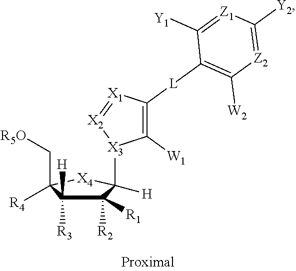

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl; Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is a McGuigan Protide having the structure

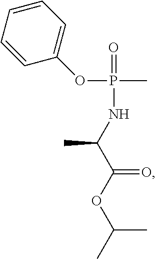 [SMILES: CC(C)OC(=O)[C@@H](C)NP(C)(=O)Oc1ccccc1]

or a pharmaceutically acceptable salt, isomer, hydrate, prodrug or solvate thereof.
In some embodiments, X₁=X₃=N and X₂ is C. In some embodiments, Z₁=Z₂=N. In some embodiments, W₁=W₂=H. In some embodiments, R₁ is CH₃, R₂ is F, R₃ is OH and/or R₄ is H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and R₁ is CH₃, R₂ is F, R₃ is OH and/or R₄ is H. In some embodiments, X₁=X₃=N, X₂ is C, and L is null. In some embodiments, R₁ is CH₃, R₂ is F, R₃ is OH and/or R₄ is H and Z₁=Z₂=N. In some embodiments, R₁ is CH₃, R₂ is F, R₃ is OH and/or R₄ is H and W₁=W₂=H. In some embodiments, R₁ is CH₃, R₂ is F, R₃ is OH and/or R₄ is H and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, R₁ is CH₃, R₂ is F, R₃ is OH and/or R₄ is H, and L is null.

In some embodiments of the first aspect, the flexible nucleoside analogue comprises at least one of:

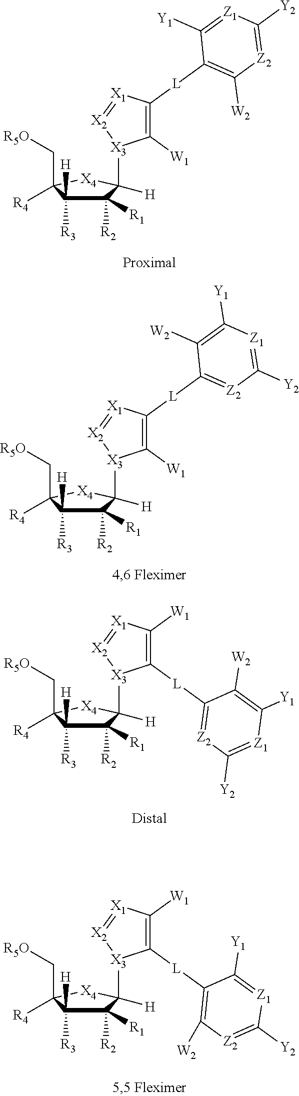

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan
ProTides, lipid phosphates, lipid esters, HepDirect, amino acid prodrugs), or a pharmaceutically acceptable salt, isomer, hydrate, prodrug or solvate thereof,
   wherein in the Proximal compound, when R₁ is CH₃, R₂ is F, R₃ is OH, R₄ is H, R₅ is a McGuigan Protide, W₁=W₂=H, Z₁=Z₂=N, L is null, X₁=X₃=N, X₂ is C, X₄ is O, and Y₂ is NH₂, Y₁ cannot be NH(CH₃).

In some embodiments, X₁=X₃=N and X₂ is C. In some embodiments, Z₁=Z₂=N. In some embodiments, W₁=W₂=H. In some embodiments, R₁ is CH₃, R₂ is F, R₃ is OH and/or R₄ is H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and R₁ is CH₃, R₂ is F, R₃ is OH and/or R₄ is H. In some embodiments, X₁=X₃=N, X₂ is C, and L is null. In some embodiments, R₁ is CH₃, R₂ is F, R₃ is OH and/or R₄ is H and Z₁=Z₂=N. In some embodiments, R₁ is CH₃, R₂ is F, R₃ is OH and/or R₄ is H and W₁=W₂=H. In some embodiments, R₁ is CH₃, R₂ is F, R₃ is OH and/or R₄ is H and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, R₁ is CH₃, R₂ is F, R₃ is OH and/or R₄ is H, and L is null.

In some embodiments of the first aspect, the flexible nucleoside analogue comprises at least one of:

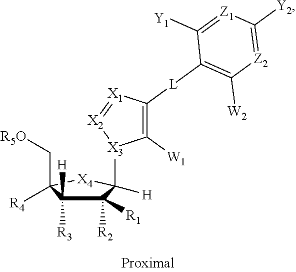

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof,
   wherein when R₁ is CH₃, R₂ is F, R₃ is OH, R₄ is H, R₅ is a McGuigan Protide, W₁=W₂=H, Z₁=Z₂=N, L is null, X₁=X₃=N, X₂ is C, X₄ is O, and Y₂ is NH₂, Y₁ cannot be NH(CH₃).
   In some embodiments, X₁=X₃=N and X₂ is C. In some embodiments, Z₁=Z₂=N. In some embodiments, W₁=W₂=H. In some embodiments, R₁ is CH₃, R₂ is F, R₃ is OH and/or R₄ is H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and R₁ is CH₃, R₂ is F, R₃ is OH and/or R₄ is H. In some embodiments, X₁=X₃=N, X₂ is C, and L is null. In some embodiments, R₁ is CH₃, R₂ is F, R₃ is OH and/or R₄ is H and Z₁=Z₂=N. In some embodiments, R₁ is CH₃, R₂ is F, R₃ is OH and/or R₄ is H and W₁=W₂=H. In some embodiments, R₁ is CH₃, R₂ is F, R₃ is OH and/or R₄ is H and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, R₁ is CH₃, R₂ is F, R₃ is OH and/or R₄ is H, and L is null.

In some embodiments of the first aspect, the flexible nucleoside analogue comprises at least one of:

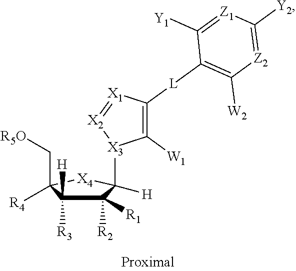

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is a McGuigan Protide having the structure

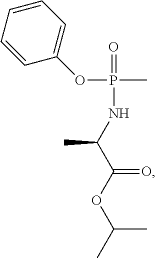 [SMILES: CC(C)OC(=O)[C@@H](C)NP(C)(=O)Oc1ccccc1]

or a pharmaceutically acceptable salt, isomer, hydrate, prodrug or solvate thereof,
wherein when R₁ is CH₃, R₂ is F, R₃ is OH, R₄ is H, R₅ is a McGuigan Protide, W₁=W₂=H, Z₁=Z₂=N, L is null, X₁=X₃=N, X₂ is C, X₄ is O, and Y₂ is NH₂, Y₁ cannot be NH(CH₃).

In some embodiments, X₁=X₃=N and X₂ is C. In some embodiments, Z₁=Z₂=N. In some embodiments, W₁=W₂=H. In some embodiments, R₁ is CH₃, R₂ is F, R₃ is OH and/or R₄ is H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and R₁ is CH₃, R₂ is F, R₃ is OH and/or R₄ is H. In some embodiments, X₁=X₃=N, X₂ is C, and L is null. In some embodiments, R₁ is CH₃, R₂ is F, R₃ is OH and/or R₄ is H and Z₁=Z₂=N. In some embodiments, R₁ is CH₃, R₂ is F, R₃ is OH and/or R₄ is H and W₁=W₂=H. In some embodiments, R₁ is CH₃, R₂ is F, R₃ is OH and/or R₄ is H and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, R₁ is CH₃, R₂ is F, R₃ is OH and/or R₄ is H, and L is null.

In some embodiments of the first aspect, the flexible nucleoside analogue comprises at least one of:

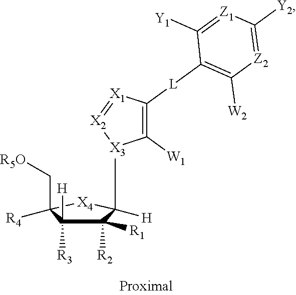

wherein
L is null;
X₁ and X₃ are each N;
X₂ is C;
X₄ is O;
W₁ and W₂ are each H;
Y₁ and Y₂ are each independently selected from H, ═O, OH, S, F, NH₂, NH-alkyl, O-alkyl, alkyl, or NH—OH;
Z₁ and Z₂ are N;
R₁ is alkyl;
R₂ is F;
R₃ is OH;
R₄ is H; and
R₅ is H, monophosphate, diphosphate, triphosphate, various prodrugs (e.g., esters, McGuigan ProTides, lipid phosphates, lipid esters, HepDirect, amino acid prodrugs),
   or a pharmaceutically acceptable salt, isomer, hydrate, prodrug or solvate thereof,
   wherein when R₁ is CH₃, R₅ is a McGuigan Protide, and Y₂ is NH₂, Y₁ cannot be NH(CH₃).

In some embodiments of the first aspect, the flexible nucleoside analogue comprises at least one of:

### (A)

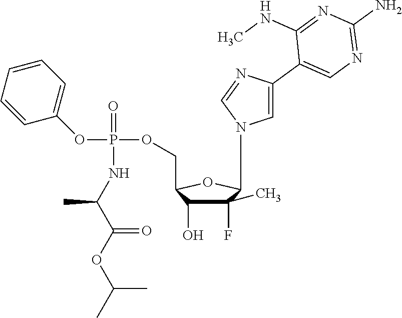

propan-2-yl-(2S)-2-(((((2R,3R,4R,5R)-5-(4-(2-amino-4-(methylamino)pyrimidin-5-yl)-1H-imidazol-1-yl)-4-fluoro-3-hydroxy-4-methyloxolan-2-yl)methyl)(phenyl)phosphono)amino) propanoate;

### (B)

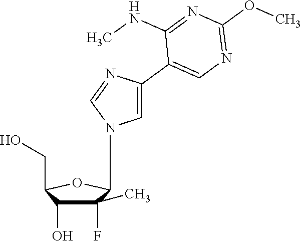

(2R,3R,4R,5R)-5-(4-(2,4-dimethoxypyrimidin-5-yl)-1H-imidazol-1-yl)-4-fluoro-2-(hydroxymethyl)-4-methyltetrahydrofuran-3-ol;

### (C)

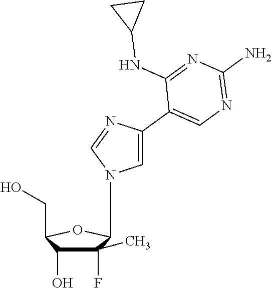

(2R,3R,4R,5R)-5-(4-(2-amino-4-(cyclopropylamino)pyrimidin-5-yl)-1H-imidazol-1-yl)-4-fluoro-2-(hydroxymethyl)-4-methyltetrahydrofuran-3-ol;

### (D)

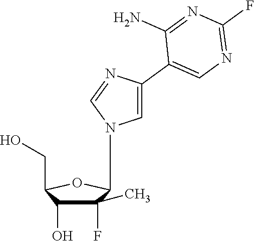

(2R,3R,4R,5R)-5-(4-(4-amino-2-fluoropyrimidin-5-yl)-1H-imidazol-1-yl)-4-fluor-2-(hydroxymethyl)-4-methyltetrahydrofuran-3-ol;

### (E)

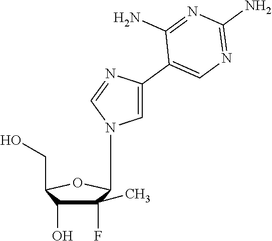

(2R,3R,4R,5R)-5-(4-(2,4-diaminopyrimidin-5-yl)-1H-imidazol-1-yl)-4-fluoro-2-(hydroxymethyl)-4-methyltetrahydrofuran-3-ol;

### (F)

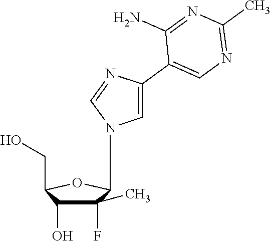

(2R,3R,4R,5R)-5-(4-(4-amino-2-methylpyrimidin-5-yl)-1H-imidazol-1-yl)-4-fluor-2-(hydroxymethyl)-4-methyltetrahydrofuran-3-ol;

### (G)

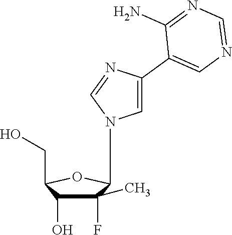

(2R,3R,4R,5R)-5-(4-(4-aminopyrimidin-5-yl)-1H-imidazol-1-yl)-4-fluor-2-(hydroxymethyl)-4-methyltetrahydrofuran-3-ol;

### (H)

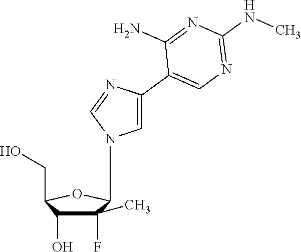

(2R,3R,4R,5R)-5-(4-(4-amino-2-(methylamino)pyrimidin-5-yl)-1H-imidazol-1-yl)-4-fluor-2-(hydroxymethyl)-4-methyltetrahydrofuran-3-ol;

### (I)

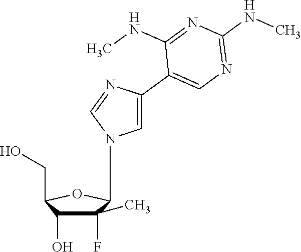

(2R,3R,4R,5R)-5-(4-(2,4-bis(methylamino)pyrimidin-5-yl)-1H-imidazol-1-yl)-4-fluor-2-(hydroxymethyl)-4-methyltetrahydrofuran-3-ol;

### (J)

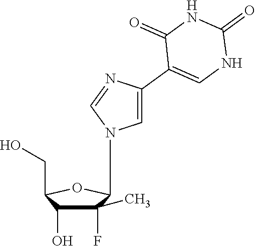

5-(1-((2R,3R,4R,5R)-3-fluoro-4-hydroxy-5-(hydroxymethyl)-3-methyltetrahydrofuran-2-yl)-1H-imidazol-4-yl)pyrimidine-2,4(1H,3H)-dione;

### (K)

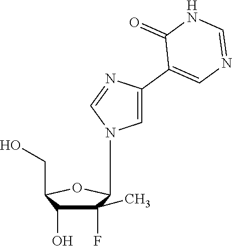

5-(1-((2R,3R,4R,5R)-3-fluoro-4-hydroxy-5-(hydroxymethyl)-3-methyltetrahydrofuran-2-yl)-1H-imidazol-4-yl)pyrimidin-4(3H)-one;

### (L)

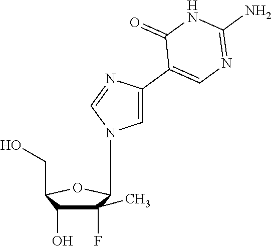

2-amino-5-(1-((2R,3R,4R,5R)-3-fluoro-4-hydroxy-5-(hydroxymethyl)-3-methyltetrahydrofuran-2-yl)-1H-imidazol-4-yl)pyrimidin-4(3H)-one; and

### (M)

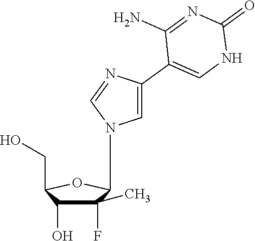

4-amino-5-(1-((2R,3R,4R,5R)-3-fluoro-4-hydroxy-5-(hydroxymethyl)-3-methyltetrahydrofuran-2-yl)-1H-imidazol-4-yl)pyrimidin-2(1H)-one
   or a pharmaceutically acceptable salt, isomer, hydrate, prodrug or solvate thereof.

In some embodiments, the flexible nucleoside analogue comprises at least one of compounds (B)-(M).

In some embodiments of the first aspect, the flexible nucleoside analogue comprises at least one of:

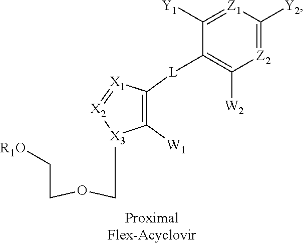

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, monophosphate, diphosphate, triphosphate, various prodrugs (e.g., esters, McGuigan ProTides, lipid phosphates, lipid esters, HepDirect, amino acid prodrugs) or —C(O)-alkyl,
   or a pharmaceutically acceptable salt, isomer, hydrate, prodrug or solvate thereof.
   In some embodiments, X₁=X₃=N and X₂ is C. In some embodiments, Z₁=Z₂=N. In some embodiments, W₁=W₂=H. In some embodiments, L is null. In some embodiments, X₁=X₃=N, X₂ is C, and Z₁=Z₂=N. In some embodiments, X₁=X₃=N, X₂ is C, and W₁=W₂=H. In some embodiments, X₁=X₃=N, X₂ is C, and L is null. In some embodiments, Z₁=Z₂=N and L is null. In some embodiments, Z₁=Z₂=N and W₁=W₂=H. In some embodiments, W₁=W₂=H and L is null. In some embodiments, X₁=X₃=N, X₂ is C, Z₁=Z₂=N, W₁=W₂=H, and L is null.

In some embodiments of the first aspect, the flexible nucleoside analogue comprises at least one of:

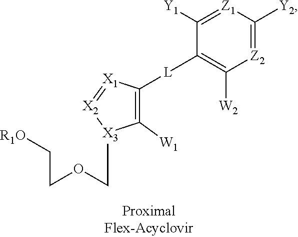

wherein
L is null;
X₁=X₃=N;
X₂ is C;
Y₁ and Y₂ are each independently selected from H, OH, S, F, NH₂, NH-alkyl, O-alkyl, or NH—OH;
W₁ and W₂ are each H;
Z₁ and Z₂ are each N; and
R₁ is H, monophosphate, diphosphate, triphosphate, various prodrugs (e.g., esters, McGuigan ProTides, lipid phosphates, lipid esters, HepDirect, amino acid prodrugs) or —C(O)-alkyl,
   or a pharmaceutically acceptable salt, isomer, hydrate, prodrug or solvate thereof.

In some embodiments of the first aspect, the flexible nucleoside analogue comprises at least one of:

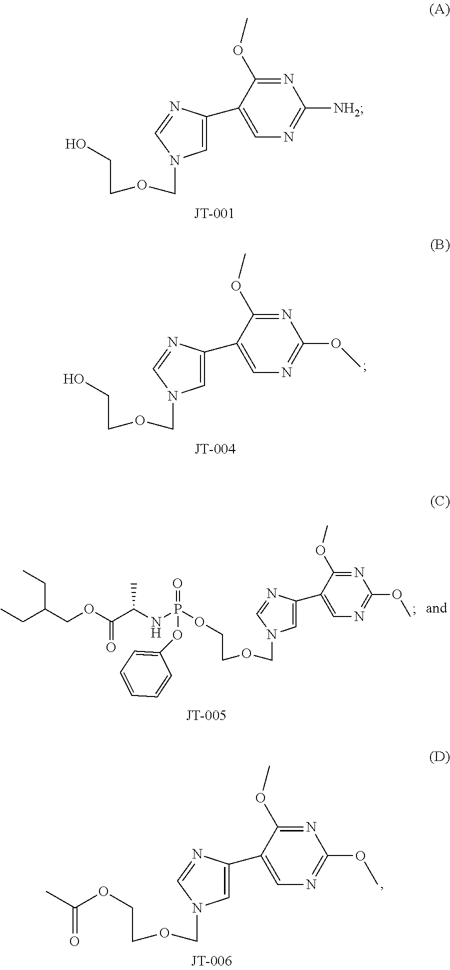

or a pharmaceutically acceptable salt, isomer, hydrate, prodrug or solvate thereof.

In a second aspect, the present invention relates to fleximer nucleoside analogues similar to Remdesivir, selected from:

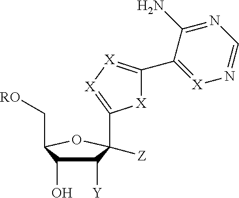

wherein
X is N or C;
Y is OH or F;
Z is H, CN, N₃ or C≡CH; and
R is H, acetoxy (OAc), monophosphate, diphosphate, triphosphate, various prodrugs such as McGuigan ProTides, Lipid phosphates, or esters, or a pharmaceutically acceptable salt, isomer, hydrate, prodrug or solvate thereof.

In some embodiments of the second aspect, the flexible nucleoside analogue comprises:

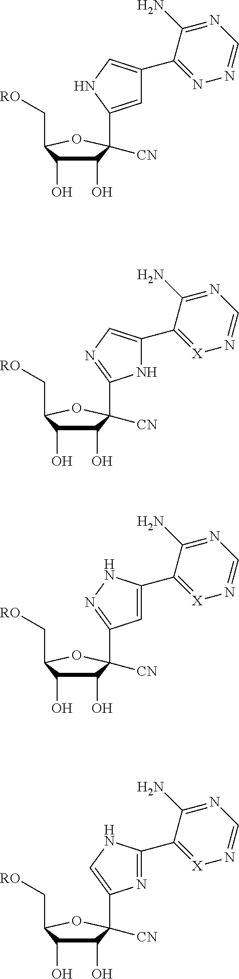

wherein
X is N or C;
R is H, acetoxy (OAc), monophosphate, diphosphate, triphosphate, various prodrugs such as McGuigan ProTides, Lipid phosphates, or esters,
or a pharmaceutically acceptable salt, isomer, hydrate, prodrug or solvate thereof.

In some embodiments of the second aspect, the flexible nucleoside analogue comprises:

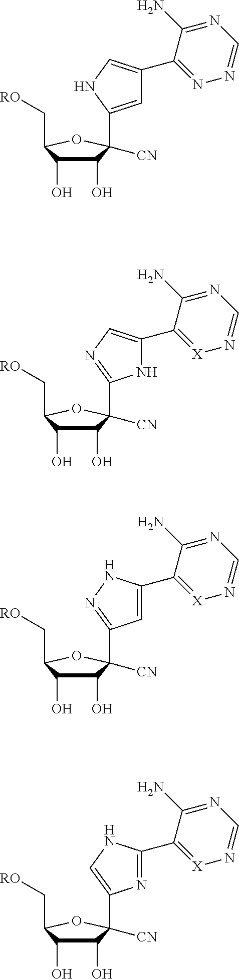

X is N or C;
R is H, acetoxy (OAc), monophosphate, diphosphate, triphosphate, various prodrugs such as McGuigan ProTides, Lipid phosphates, or esters,
or a pharmaceutically acceptable salt, isomer, hydrate, prodrug or solvate thereof.

### Compostions Comprising the Flexible Nucleoside Analogues Described Herein

The fleximer nucleoside analogues described herein may be administered in various ways and in various forms. In some embodiments, the fleximer nucleoside analogues described herein may be administered systemically, orally (including buccal or sublingual), topically, by inhalation (or spray) or by injection (e.g., intravenously, intramuscularly, subcutaneously, intravasularly, intrathecally, intradermally, intra-arterially). For the injections, the fleximer nucleoside analogues are generally present in the form of liquid suspensions, which can be injected by means of syringes or infusions, for example. In this regard, the fleximer nucleoside analogues described herein are generally dissolved in solutions that can be buffered, isotonic, physiological, and/or saline, and which are compatible with pharmaceutical use and known to those skilled in the art.

Accordingly, in a third aspect, a composition formulated for therapeutic use is described, wherein the composition comprises, consists of, or consists essentially of at least one fleximer nucleoside analogue of the first or second aspect, and at least one pharmaceutically acceptable carrier, excipient, or diluent. The pharmaceutical composition will typically be formulated in a format suitable for administration to the subject, for example, as a syrup, elixir, tablet, troche, lozenge, hard or soft capsule, pill, suppository, oily or aqueous suspension, dispersible powder or granule, emulsion, injectable, solution, sustained release formulation, or aerosol. Some examples of acceptable excipients are those that are non-toxic, will aid administration to the patient, and do not adversely affect the therapeutic benefit of the fleximer nucleoside analogue compound. Such excipient may be a solid, liquid, semi-solid or, in the case of an aerosol composition, a gaseous excipient, that is generally understood by one of skill in the art. In some embodiments, solid pharmaceutical excipients include, but are not limited to, starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients include, but are not limited to, glycerol, propylene glycol, water, ethanol, and various oils, including those of petroleum, animal, vegetable, or synthetic origin, e.g., peanut oil, soybean oil, mineral oil, sesame oil, etc. Preferred liquid carriers, particularly for injectable solutions, include, but are not limited to, water, saline, aqueous dextrose, and glycols. Liquid compositions may contain one or more agents or carriers chosen from dispersants, solubilizing agents, stabilizers, preservatives, and any combination thereof. Agents or carriers which can be used in liquid and/or injectable formulations include, but are not limited to, methylcellulose, hydroxymethylcellulose, carboxymethylcellulose, polysorbate 80, mannitol, gelatin, lactose, vegetable oils, and acacia. Other suitable pharmaceutical excipients and their formulations are described in Remington's Pharmaceutical Sciences, edited by E. W. Martin (Mack Publishing Company, 18th ed., 1990).

The amount of a fleximer nucleoside analogue of the first or second aspect in the composition can vary within the full range employed by those skilled in the art. In some embodiments, a composition may contain, on a weight percent (wt %) basis, from about 0.01-99.99 wt % of a fleximer nucleoside analogue, based on the total weight of the composition, with the balance being one or more suitable pharmaceutical excipients. In some embodiments, the composition comprises an amount of a fleximer nucleoside analogue of the first or second aspect of between 5 μg and 1000 mg, preferably between 1 and 500 mg, preferably between 5 and 100 mg. The ratio between the amounts by weight of a fleximer nucleoside analogue and of pharmaceutically acceptable carrier is between 5/95 and 95/5, preferably between 20/80 and 80/20.

The fleximer nucleoside analogues may be the only active ingredients in the composition of the third aspect, or they may be combined with other active ingredients. Accordingly, in some embodiments of the third aspect, the composition may comprise, consist of, or consist essentially of at least one fleximer nucleoside analogue of the first or second aspect, at least one other pharmaceutically active agent, e.g., at least one other medicament used for the treatment of viral infection, and at least one pharmaceutically acceptable carrier, excipient, or diluent. In some embodiments, the composition may comprise, or be combined with, one or more other antivirals or antiretrovirals (e.g., nucleoside or nucleotide and non-nucleoside inhibitors, protease inhibitors, entry inhibitors, etc.).

### Methods of Using

In a fourth aspect, a method for treating and/or preventing a filoviral, flaviviral, herpesviral, alphaviral, polyomaviral, enteroviral, matonaviral, phenuiviral, and/or coronaviral infection is described, comprising the administration, to a patient, of an effective amount of at least one fleximer nucleoside analogue of the first or second aspect or of a composition including same. In some embodiments, the fleximer nucleoside analogues, as active agents, will be administered in a therapeutically effective amount by any of the accepted modes of administration for agents that serve similar utilities. The “effective amount” will be an amount of a fleximer nucleoside analogue, as described herein, that would be understood by one skilled in the art to provide therapeutic benefits. The active agent can be administered once a week, two or more times per week, once a day, or more than once a day. As indicated above, all of the factors to be considered in determining the effective amount will be well within the skill of the attending clinician or other health care professional. In some embodiments, therapeutically effective amounts of a fleximer nucleoside analogue, as described herein, may range from approximately 0.05 to 50 mg per kilogram body weight of the subject per day; preferably about 0.1-25 mg/kg/day, more preferably from about 0.5 to 10 mg/kg/day. Thus, for administration to a 70 kg person, the dosage range would most preferably be about 35-700 mg per day.

In some embodiments of the fourth aspect, a method for treating and/or preventing a viral infection in a subject is described, wherein the viral infection is caused by at least one of a coronavirus, a herpesvirus, an alphavirus, a polyomavirus, an enterovirus, a filovirus, a matonavirus, a phenuivirus, and/or a flavivirus, comprising administration, to the subject, of a therapeutically effective amount of at least one fleximer nucleoside analogue selected from the first or second aspects described herein, or a composition of the third aspect described herein. In some embodiments, a therapeutically effective amount of the fleximer nucleoside analogue is from 0.05 to 50 mg per kilogram body weight of the subject per day. In some embodiments, the fleximer nucleoside analogue is present in, and administered as, a composition, as described in the third aspect herein. In some embodiments, the viral infection is caused by a coronavirus. In some embodiments, the viral infection is caused by a herpesvirus. In some embodiments, the viral infection is caused by an alphavirus. In some embodiments, the viral infection is caused by a polyomavirus. In some embodiments, the viral infection is caused by an enterovirus. In some embodiments, the viral infection is caused by a filovirus. In some embodiments, the viral infection is caused by a matonavirus. In some embodiments, the viral infection is caused by a phenuivirus. In some embodiments, the viral infection is caused by a flavivirus. In some embodiments, the method of administration is selected from systemically, orally, buccally, sublingually, topically, by inhalation, by spraying, intravenously, intramuscularly, subcutaneously, intrathecally, intradermally, intravascularly or intra-arterially.

In some embodiments of the fourth aspect, a method of treating and/or reducing the effects of Epstein Barr virus in a subject in need of such treatment is described, the method comprising administering to the subject a therapeutically effective amount of a fleximer nucleoside analogue selected from:

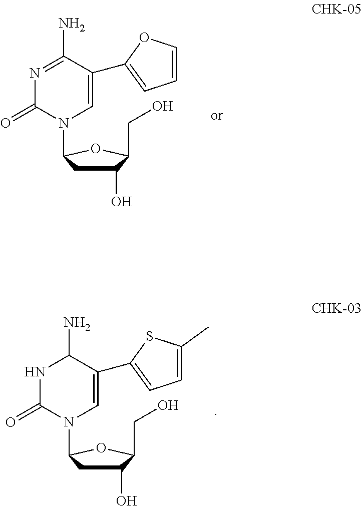

and a pharmaceutically acceptable salt, isomer, hydrate, prodrug or solvate thereof. In some embodiments, a therapeutically effective amount of the fleximer nucleoside analogue is from 0.05 to 50 mg per kilogram body weight of the subject per day.

In some embodiments of the fourth aspect, a method of treating and/or reducing the effects of Epstein Barr virus in a subject in need of such treatment is described, the method comprising administering to the subject a therapeutically effective amount of a fleximer nucleoside analogue selected from:

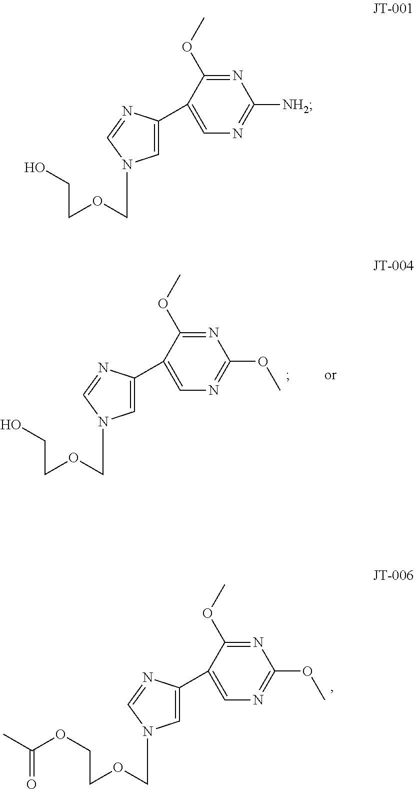

and a pharmaceutically acceptable salt, isomer, hydrate, prodrug or solvate thereof. In some embodiments, a therapeutically effective amount of the fleximer nucleoside analogue is from 0.05 to 50 mg per kilogram body weight of the subject per day.

In some embodiments of the fourth aspect, a method of treating and/or reducing the effects of a coronavirus, enterovirus, Chikungunya virus, Ebola virus and/or Dengue virus in a subject in need of such treatment is described, the method comprising administering to the subject a therapeutically effective amount of a fleximer nucleoside analogue having the following structure:

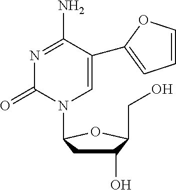

and a pharmaceutically acceptable salt, isomer, hydrate, prodrug or solvate thereof. In some embodiments, a therapeutically effective amount of the fleximer nucleoside analogue is from 0.05 to 50 mg per kilogram body weight of the subject per day.

In some embodiments of the fourth aspect, a method of treating and/or reducing the effects of Ebola virus in a subject in need of such treatment is described, the method comprising administering to the subject a therapeutically effective amount of a fleximer nucleoside analogue selected from:

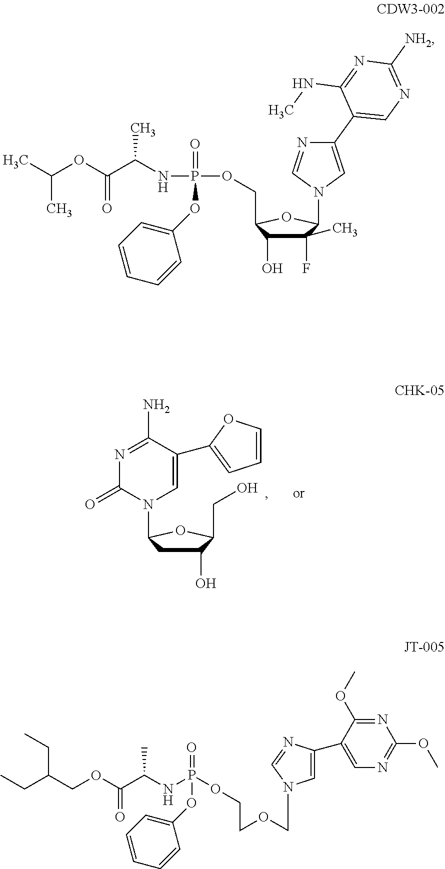

and a pharmaceutically acceptable salt, isomer, hydrate, prodrug or solvate thereof. In some embodiments, a therapeutically effective amount of the fleximer nucleoside analogue is from 0.05 to 50 mg per kilogram body weight of the subject per day.

In some embodiments, the viral infection is caused by a coronavirus selected from human coronaviruses (HCoV), Severe Acute Respiratory Syndrome Coronavirus (SARS-CoV), SARS-CoV-2, and Middle East respiratory syndrome (MERS), and mutants thereof. In some embodiments, the viral infection is caused by a herpesvirus selected from herpes simplex virus 1, herpes simplex virus 2, varicella-zoster virus, Epstein-Barr virus, cytomegalovirus, Human herpesvirus-6, Human herpesvirus-7, and Kaposi's sarcoma herpes virus. In some embodiments, the viral infection is caused by an alphavirus selected from eastern equine encephalomyelitis (EEE). Venezuelan equine encephalomyelitis (VEE), and western equine encephalomyelitis (WEE). In some embodiments, the viral infection is caused by a polyomavirus. In some embodiments, the viral infection is caused by an enterovirus selected from echovirus and coxsackievirus. In some embodiments, the viral infection is caused by a filovirus selected from Zaire Ebola virus, Sudan Ebola virus, Reston Ebola virus, Cote d'Ivoire Ebola virus and Marburg virus. In some embodiments, the viral infection is caused by a matonavirus selected from Rubella, Rustrela, and Ruhugu. In some embodiments, the viral infection is caused by a phenuivirus selected from Rift Valley Fever virus. In some embodiments, the viral infection is caused by a flavivirus selected from the group consisting of yellow fever virus, Apoi virus, Aroa virus, Bagaza virus, Banzi virus, Bouboui virus, Bukalasa bat virus, Cacipacore virus, Carey Island virus, Cowbone Ridge virus, Dakar bat virus, dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Phenh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, tick-borne encephalitis virus, Tyuleniy virus, Uganda S virus, Usutu virus, Wesselsbron virus, West Nile virus, Yaounde virus, Yokose virus, Zika virus, cell fusing agent virus and Tamana bat virus.

In some embodiments of the fourth aspect, a method for binding to, or interacting with, natural and/or mutated polymerases of a filovirus, flavivirus, alphaviruses, polyomaviruses, enteroviruses, herpesvirus, matonavirus, phenuivirus or coronavirus to induce inhibition activity of said virus is described, the method comprising administering to the subject a therapeutically effective amount of at least one fleximer nucleoside analogue of the first or second aspect, or a composition including same. Additional target enzymes include, but not limited to, various viral methyltransferases for all of the viruses, including the exonuclease (for CoVs), the RNA dependent RNA polymerases (RdRps) for all of the viruses, and the NiRAN (RdRp associated nucleotidyl transferase domain) for CoVs.

In another aspect, a use of at least one fleximer nucleoside analogue of the first or second aspect, or a composition including same, in a medicament for medicine is described. In a more specific embodiment hereof, said use as a medicine is for the prevention or treatment of a filovirus, herpesvirus, flavivirus, alphavirus, polyomavirus, enterovirus, matonavirus, phenuivirus and/or coronavirus in a subject, mammal or human. In some embodiments, a therapeutically effective amount of the fleximer nucleoside analogue is from 0.05 to 50 mg per kilogram body weight of the subject per day. In some embodiments, the use as a medicine is for the prevention or treatment of a coronavirus, SARS and MERS-CoV in a subject. In some embodiments, the use as a medicine is for the prevention or treatment of an infection of a COVID-19 coronavirus or a mutant thereof in a subject. In some embodiments, the use as a medicine is for the prevention or treatment of viruses including, but not limited to, filoviruses such as Ebola, Marburg and Sudan, and flaviviruses such as dengue, zika, yellow fever, and tickborne encephalitis, in a subject.

In another aspect, the manufacture of a medicament comprising at least one fleximer nucleoside analogue of the first or second aspect, or a composition including same, for the treatment of a coronavirus, herpesvirus, alphaviruses, enteroviruses, filovirus, matonavirus, phenuivirus and/or flavivirus is described.

In another aspect, novel intermediates or prodrugs which are useful for preparing at least one fleximer nucleoside analogue of the first or second aspect or converted to active agents in vivo is described. Prodrugs are selected and prepared in order to improve some selected property of the molecule, such as water solubility or ability to cross a membrane, temporarily. Most common (biologically labile) functional groups utilized in prodrug design include, but are not limited to, carbonates, esters, amino acyl esters, amides, carbamates, oximes, imines, ethers, or phosphates.

In a still further aspect, the present application provides for a method of treating a filovirus, flavivirus, alphaviruses, polyomaviruses, enteroviruses, herpesvirus, matonavirus, phenuivirus or coronavirus in a patient, comprising administering to said patient a therapeutically effective amount of at least one fleximer nucleoside analogue of the first or second aspect, or a composition including same, and at least one additional therapeutic agent having anti-viral properties.

In another aspect, a method for treating a viral infection comprises the administration, to a patient, of an effective amount of at least one fleximer nucleoside analogue of the first or second aspect, or a composition including same, is described. In some embodiments, the fleximer nucleoside analogue can be a prodrug or otherwise capable of releasing the active ingredient after in vivo metabolism.

In another aspect, a method for treating a CoV viral infection is described, comprising the administration, to a patient, of an effective amount of at least one fleximer nucleoside analogue of the first or second aspect, or a composition including same. The fleximer nucleoside analogue can further be a prodrug or in form of capable of releasing the active ingredient after in vivo metabolism.

In another aspect, a cell infected with a virus or to be infected with the virus is contacted with at least one fleximer nucleoside analogue of the first or second aspect, or a composition including same, wherein the virus is selected from a filovirus, flavivirus, alphaviruses, polyomaviruses, enteroviruses, herpesvirus, matonavirus, phenuivirus or coronavirus. In some embodiments, the amount of the at least one fleximer nucleoside analogue used is from about 1 μg/ml to about 40 μg/ml, and more preferably, from about 3 sg/ml to about 20 μg/ml.

In another aspect, methods for synthesis, analysis, separation, isolation, purification, characterization, and testing of the compounds of the first or second aspect are provided.

The features and advantages of the invention are more fully illustrated by the following non-limiting examples, wherein all parts and percentages are by weight, unless otherwise expressly stated.

### Example 1

### Testing Methods and Results

### Primary CPE Test Method (USU).

Four-concentration CPE inhibition assays are performed on confluent or near-confluent cell culture monolayers in 96-well plates. Cells are maintained in MEM or DMEM supplemented with FBS as required for each cell line. For antiviral assays the same medium is used but with FBS at 2% or less and supplemented with 50 μg/ml gentamicin (10 μg/mL EDTA and 1 IU/mL trypsin are added for influenza, and 25 mM MgCl₂ for rhinovirus and enterovirus-D68). The test compound is prepared for testing at 4-concentrations, usually 100, 10, 1.0, and 0.1 μg/ml or μM. Higher or lower concentrations are used at the direction of the sponsor/COR. Five microwells are used per dilution: three for infected wells and two for uninfected toxicity evaluation wells that are run in parallel. Controls for the experiment consist of six microwells that are infected but not treated (virus controls) and six that are untreated (cell controls). The virus control and cell control wells are on every 96-well test plate. A known active drug is tested in parallel with each assay run as a positive control, using the same method as is used for test compounds.

Growth medium is removed from the 96-well plates of cells, then the test compound is applied in 0.1 ml volume to wells at 2× concentration. Virus, normally <100 50% cell culture infectious doses (CCID₅₀) in 0.1 ml volume, is added to wells designated for virus infection. Medium devoid of virus is placed in toxicity control wells and cell control wells. Plates are incubated at 37° C. (33° C. for enterovirus-D68) with 5% CO₂ until maximum CPE is observed microscopically in virus control wells. Plates are then stained with 0.011% neutral red for approximately two hours at 37° C. with 5% CO₂. The neutral red medium is removed by complete aspiration, and the cells rinsed with phosphate buffered saline (PBS) to remove residual dye. The PBS is completely removed, and the incorporated neutral red is eluted with 50% Sorensen's citrate buffer/50% ethanol for at least 30 minutes. Neutral red dye penetrates living cells, thus, the more intense the red color, the larger the number of viable cells present. The dye content in each well is quantified by optical density (OD) on a spectrophotometer at 540 nm wavelength. The OD for each set of wells is converted to a percentage compared to untreated control wells using a Microsoft Excel™ computer-based spreadsheet. Infected wells are normalized to the virus control. The 50% effective (EC₅₀, virus-inhibitory) concentrations and 50% cytotoxic (CC₅₀, cell-inhibitory) concentrations are then calculated by regression analysis. The quotient of CC₅₀ divided by EC₅₀ gives the selectivity index (SI₅₀) value. The percent CPE in each well may also be read microscopically, calculated as above, and reported as a second data set from the same plate for verification.

Compounds showing SI₅₀ values 10 are considered active and merit further investigation at the discretion of the COR. For certain viruses that are difficult to inhibit, such as West Nile virus, compounds with SI₅₀ values≥5 may be considered for further testing as well. One set of data is provided to NIH for each compound tested and includes ECs, CC₅₀, and SI₅₀ obtained from the neutral red assay. For fastidious viruses that don't cause observable CPE (e.g., LCMV, Heartland, and SFTS), the cell lysate can be harvested and titrated as described in the secondary assay below. An EC₉₀ is then reported instead of an EC₅₀ value, and visual readings are used to determine the toxicity (CC₅₀).

### Secondary Assay Method: CPE and Virus Yield Reduction (VYR) Test (USU).

Active compounds are submitted for secondary assays at the discretion of the COR. The secondary assay confirms the CPE assay result, with the principal assessment being virus yield reduction (VYR). It employs a similar method as described for the primary assay in section 2C.8.1, with the differences noted in this section. The secondary assay is run independently of the primary test with cells, culture media, infectious virus solution, and test compound dilutions all newly prepared for this assay. Eight half-log₁₀ concentrations are tested for antiviral activity and cytotoxicity. After sufficient virus replication occurs (normally 3 days post infection), a sample of supernatant is taken from each infected well (three replicate wells are pooled) and titrated immediately (as with RSV) or frozen and stored for virus titration at a later time. Alternately, a separate plate may be prepared, and the plate may be frozen and then thawed to release intracellular or cell-associated virus to test the cell lysate rather than supernatant fluid. After maximum CPE is observed, the plates are stained with neutral red dye as described above to generate the neutral red EC₅₀, CC₅₀, and SI₅₀ values. Uninfected wells are tested in parallel for compound toxicity as in the primary assay explained above. The positive control compound is evaluated in parallel with each test.

The second step is to determine the virus titer in the supernatant or lysate samples. Samples from triplicate wells, collected as described above, are pooled and tested by endpoint dilution. This is a direct determination virus produced in the presence of the test compound compared to virus from untreated, infected controls. This is accomplished by making log₁₀ dilutions of the pooled samples and plating each dilution on 3 or 4 microwells with fresh monolayers of cells in 96-well plates. Plates are incubated until maximum CPE is observed, then each well is scored for presence (+) or absence (−) of virus and the virus titer calculated using the Reed-Meunch method (1938). Plotting the log of the inhibitor concentration versus log₁₀ of virus produced at each concentration allows calculation of the 90% (one log₁₀ reduction) effective concentration by linear regression. Dividing CC₅₀ toxicity obtained in the CPE assay by the EC₉₀ gives the SI₉₀ value (so named because it is derived from a 90% virus-inhibitory value). Two sets of data are provided to NIH: (1) EC₅₀, CC₅₀, and SI₅₀ obtained from the neutral red assay, and (2) EC₉₀ obtained from the VYR assay, CC₅₀, and SI₉₀. For fastidious viruses that do not cause notable CPE, the EC₉₀ and CC₅₀ will be reported, but no EC₅₀ value.

### Primary Plaque Reduction Assay Method for BSL-4 Viruses (Lassa Fever, Ebola, Marburg, and Nipah).

Confluent or near-confluent cell culture monolayers in 12-well disposable cell culture plates are prepared. 24- or 48-well plates could be used for EBOV and MARV as well. Cells are maintained in MEM supplemented with 5 to 10% FBS. The test compound is prepared at four log₁₀ final concentrations, usually 100, 10, 1.0, and 0.1 μg/ml or μM (depending upon the sponsor's preference) in 2×MEM. Lower concentrations are used when insufficient compound is provided, after discussion with the PI and COR. The virus control and cell control are run in parallel with each tested compound. Further, a known active drug is tested as a positive control drug (e.g., favipiravir) using the same experimental set-up as described for the virus and cell control. The positive control is tested with each test run. Test compounds and positive controls are typically tested in biological triplicates. The assay is initiated by first removing growth media from the 12-well plates of cells, and infecting cells with 0.01 MOI of virus or about 50 to 100 plaque forming units (pfu). Cells are incubated for 60 min: 100 μl inoculum/well, at 37° C., 5% CO₂ with constant gentle rocking. Virus inoculum is removed, cells washed and overlaid with either 1% agarose, 1.8% Tragacanth, or 1% methylcellulose diluted 1:1 with 2×MEM and supplemented with 2 to 5% FBS, 1% penicillin/streptomycin, and with the corresponding drug concentration. Cells are incubated at 37° C. with 5% CO₂ for 5 (Lassa fever), 10-12 (Ebola, Marburg), or 2-3 (Nipah) days. The overlay is then removed, and plates stained with 0.05% crystal violet in 10% buffered formalin for approximately twenty minutes at room temperature. The plates are then washed, dried, and the number of plaques counted. The number of plaques in each set of compound dilution is converted to a percentage relative to the untreated virus control. The 50% effective (EC₅₀, virus-inhibitory) concentrations are then calculated by linear regression analysis. Cytotoxicity is evaluated in parallel to the actual primary PR assay.

The cytotoxicity assay (In vitro Toxicology Assay Kit, Neutral red based; Sigma) is being performed in 96-well plates following the manufacturer's instructions. Briefly, growth medium is removed from confluent cell monolayers and replaced with fresh medium (total of 100 μl) containing the test compound with the concentrations as indicated for the primary assay. Control wells will contain medium with the positive control or medium devoid of compound. Wells without cells and growth medium only serve as blanks. A total of up to five replicates are performed for each condition. Plates are then incubated for 2 to 12 days (depending on the virus used) at 37° C. with 5% CO₂. The plates are then stained with 0.033% neutral red for approximately two hours at 37° C. in a 5% CO₂ incubator. The neutral red medium is removed by complete aspiration, and the cells rinsed with phosphate buffered saline (PBS) to remove residual dye. The PBS is completely removed, and the incorporated neutral red is eluted with 1% acetic acid/50% ethanol for at least 10 minutes. Neutral red dye penetrates into living cells, thus, the more intense the red color, the more viable cells are present in the wells. The dye content in each well is quantified using a 96-well spectrophotometer at 540 nm wavelength and 690 nm wavelength (background reading). The 50% cytotoxic (CC₅₀, cell-inhibitory) concentrations are then calculated by linear regression analysis. In addition, cytotoxic effects may be visually evaluated in infected cell monolayers of the plaque assay test, and these may be used to calculate the CC₅₀ value when this is more applicable. The quotient of CC₅₀ divided by EC₅₀ gives the selectivity index (SI₅₀) value. The positive control compound is evaluated in parallel in each test. One set of data is provided to NIH for each compound tested: EC₅₀, CC₅₀, and SI₅₀ obtained from the plaque reduction assay.

### Secondary VYR Assay for BSL-4 Viruses (Lassa Fever, Ebola, Marburg, and Nipah).

The secondary assay involves similar methodology to what is described in the previous paragraphs using 12, 24, or 48-well plates of cells. The differences are noted in this section. The secondary assay is run independently of the primary test by using fresh cells, fresh culture medium, freshly prepared virus (from frozen stock), and newly prepared compound dilutions. The test compound is prepared at eight half-log₁₀ final concentrations, usually 100, 32, 10, 3.2, 0.1, and 0.032 μg/ml or μM (depending upon the sponsor's preference). Lower concentrations are used when insufficient compound is provided upon approval of the PI. Test compound is applied in 0.2 to 1 ml (depending on the plate type used) of total volume. Tissue culture supernatant (TCS) aliquots are collected at appropriate time points and then be used to determine the compounds inhibitory effect on virus replication. Virus that was replicated in the presence of test compound is titrated and compared to virus from untreated, infected controls. For titration of TCS, serial ten-fold dilutions are prepared and used to infect fresh monolayers of cells. Cells are overlaid with overlay media as described in the previous section and the number of plaques is determined. Test compounds and positive controls are typically tested in biological triplicates. The viral titer in each set of compound dilution is converted to a percentage relative to the untreated virus control. Plotting the log₁₀ of the inhibitor concentration versus log virus titer or percentage of virus produced at each concentration allows calculation of the 90% (one log₁₀) effective concentration by linear regression. Cytotoxicity is determined in uninfected cells as described in the previous section, and 50% cytotoxic (CC₅₀). One set of data is provided to NIH for each compound tested: EC₉₀, CC₅₀, and SI₉₀ obtained from the virus yield reduction assay. The positive control compound is evaluated in parallel in each test.

### Antiviral Materials and Methods

### Cell Culture and Virus Strains.

Human foreskin fibroblast (HFF) cells prepared from human foreskin tissue were obtained from the University of Alabama at Birmingham tissue procurement facility with approval from its IRB. The tissue was incubated at 4° C. for 4 h in Clinical Medium consisting of minimum essential media (MEM) with Earl's salts supplemented with 10% fetal bovine serum (FBS) (Hyclone, Inc. Logan UT), L-glutamine, fungizone, and vancomycin. Tissue is then placed in phosphate buffered saline (PBS), minced, rinsed to remove the red blood cells, and resuspended in trypsin/EDTA solution. The tissue suspension is incubated at 37° C. and gently agitated to disperse the cells, which are collected by centrifugation. Cells are resuspended in 4 ml Clinical Medium and placed in a 25 cm² flask and incubated at 37° C. in a humidified CO₂ incubator for 24 h. The media is then replaced with fresh Clinical Medium, and the cell growth is monitored daily until a confluent monolayer has formed. The HFF cells are then expanded through serial passages in standard growth medium of MEM with Earl's salts supplemented with 10% FBS, L-glutamine, penicillin, and gentamycin. The cells are passaged routinely and used for assays at or below passage 10 (1,2). COS7, C-33 A, Guinea Pig Lung, and Mouse embryo fibroblast cells were obtained from ATCC and maintained in standard growth medium of MEM with Earl's salts supplemented with 10% FBS, L-glutamine, penicillin, and gentamycin.

Akata cells were kindly provided by John Sixbey (Louisiana State University, Baton Rouge, LA). BCBL-1 cells were obtained through the NIH AIDS Research and Reference Reagent Program, Division of AIDS, NIAID, NIH. Molt-3 cells were obtained from Scott Schmid at the Centers for Disease Control and Prevention, Atlanta, GA. Lymphocytes are maintained routinely in RPMI 1640 (Mediatech, Inc., Herndon, VA) with 10% FBS, L-glutamine and antibiotics and passaged twice a week, as described previously (Keith et al., 2018; Prichard et al., 2011).

The E-377 strain of HSV-1 (herpes simplex virus-1) was a gift of Jack Hill (Burroughs Wellcome). The HCMV strain AD169, HSV-2 strain G (herpes simplex virus-2), AdV5 strain Adenoid 75, GPCMV strain 22122 and MCMV (cytomegalovirus) strain Smith were obtained from the American Type Culture Collection (ATCC, Manassas, VA). The Copenhagen strain of VACV (vaccinia virus) and Brighton strain, CPXV (Cowpox Virus) were kindly provided by John W. Huggins (Department of Viral Therapeutics, Virology Division, United States Army Medical Research Institute of Infections Disease). VZV (varicella zoster virus), strain Ellen, the polyomavirus BK virus Gardner strain (BKV) and JC virus (JCV) MAD4 strain were obtained from the ATCC. Akata cells latently infected with EBV were obtained from John Sixbey. The Z₂₉ strain of HHV-6B (human herpesvirus) was a gift of Scott Schmid at the Centers for Disease Control and Prevention, Atlanta GA. HHV-8 (human herpesvirus) was obtained as latently infected BCBL-1 cells through the NIH AIDS Research and Reference Reagent Program.

### Antiviral Assays.

Each experiment that evaluates the antiviral activity of the compounds includes both positive and negative control compounds to ensure the performance of each assay. Concurrent assessment of cytotoxicity is also performed for each study in the same cell line and with the same compound exposure (see below).
i. CPE assays for HSV-1, HSV-2, VZV, HCMV (Human cytomegalovirus), MCMV (Murine cytomegalovirus), GPCMV (Guinea Pig cytomegalovirus), AdV (Adenovirus), VACV, CPXV. Assays were performed in monolayers as described (Hartline et al., 2018). Briefly, cells were seeded in 384 well plates and incubated for 24 h to allow the formation of confluent monolayers. Dilutions of test drug were prepared directly in the plates and the monolayers infected at a predetermined MOI based on virus used. After incubation, cytopathology was determined by the addition of CellTiter-Glo (CTG) reagent. Concentrations of test compound sufficient to reduce CPE by 50% (EC₅₀) or decrease cell viability by 50% (CC₅₀) were interpolated using standard methods in Microsoft Excel. For MCMV, the assays were run in 96 well plates in mouse embryo fibroblast cells, with drug dilutions performed as above. After a 7 d incubation, DNA was extracted and a qPCR was run to calculate drug efficacy using primers 5′-TCA GCC ATC AAC TCT GCT ACC AAC-3′ (SEQ ID NO: 1), 5′-ATC TGA AAC AGC CGT ATA TCA TCT TG-3′ (SEQ ID NO: (SEQ ID NO: 2), and probe 5′-TTC TCT GTC AGC TAG CCA ATG ATA TCT TCG AGC-3′(SEQ ID NO: 3). Toxicity was measured using CTG as above. For GPCMV, the assays were run in 384 well plates in Guinea Pig Lung cells, with drug dilutions performed as above. After a 7 d incubation, DNA was extracted and a qPCR was run to calculate drug efficacy using primers 5′-GAGGTCGAGAAGCTGATATTGG-3′ (SEQ ID NO: 4), 5′-GTCTCTTCCTATGCGGGTTATC-3′(SEQ ID NO: 5), and probe 5′-ACGTCACTITGAGGGCCAACTGAT-3′ (SEQ ID NO: 6). Toxicity was measured using CTG as above.
ii. Plaque reduction assays for HSV-1, HSV-2, VZV, VACV, CPXV. Monolayers of HFF cells were prepared in six-well plates and incubated at 37° C. for 2 d to allow the cells to reach confluency. Media was then aspirated from the wells and 0.2 ml of virus was added to each of three wells to yield 20-30 plaques in each well. The virus was allowed to adsorb to the cells for 1 h and the plates were agitated every 15 minutes. Compounds were diluted in assay media consisting of MEM with Earl's salts supplemented with 2% FBS, L-glutamine, penicillin, and gentamycin. Diluted drug was added to duplicate wells and the plates were incubated for various times, depending on the virus used. For VACV and CPXV, dilutions of 2× drug was added to an equal volume of 1% agarose. For HSV-1 and -2, the monolayers were then stained with 1% crystal violet in 20% methanol and the unbound dye removed by washing with dH₂O. For all other assays, the cell monolayer was stained with 1% Neutral Red solution for 4-6 h then the stain was aspirated, and the cells were washed with PBS. For all assays, plaques were enumerated using a stereomicroscope and the concentration of compound that reduced plaque formation by 50% (EC₅₀) was interpolated from the experimental data.
iii. Yield reduction assays for HSV-1, HSV-2, HCMV, VACV, CPXV and AdV. Monolayers of HFF cells were prepared in 96-well plates and incubated at 37° C. for 1 d to allow the cells to reach confluency. Media was then aspirated from the wells and cells infected at a high MOI. At 1 h following infection, the inocula were removed and the monolayers rinsed with fresh media. Compounds were then diluted in assay media consisting of MEM with Earl's salts supplemented with 2% FBS, L-glutamine, penicillin, and gentamycin. Drug dilutions were added to the wells and the plates were incubated for various times, depending on the virus used and represents a single replication cycle for the virus. A duplicate set of dilutions were also performed but remained uninfected to serve as a cytotoxicity control and received equal compound exposure. Supernatants from each of the infected wells were subsequently titered using CTG in a TCID₅₀ assay to quantify the progeny virus. For HCMV only, viral yield in the supernatants from the infected cells were titered using qPCR with HCMV primers 5′-AGG TCT TCA AGG AAC TCA GCA AGA-3′ (SEQ ID NO: 7), 5′-CGG CAA TCG GTT TGT TGT AAA-3′(SEQ ID NO: 8), and probe 5′-CCG TCA GCC ATT CTC TCG GC-3′(SEQ ID NO: 9). For cytotoxicity controls, cytotoxicity was assessed using CTG according to the manufacturer's suggested protocol. For all assays, the concentration of compound that reduced virus titer by 90% (EC₉₀) was interpolated from the experimental data, iv. Secondary Assay for GPCMV. Compounds that were positive in the CPE assay were confirmed in a similar assay in 96-well plates according to established laboratory protocols with the compounds added 1 h post infection to identify compounds that inhibit early stages of replication including adsorption and penetration. Genome copy number was determined by qPCR using primers 5′-GAG GTC GAG AAG CTG ATA TG G-3′ (SEQ ID NO: 10), 5′-GTC TCT TCC TAT GCG GGT TAT C-3′ (SEQ ID NO: 11), and probe 5′-ACG TCA CTT TGA GGG CCA ACT GAT-3′ (SEQ ID NO: 12).
v. Secondary assay for MCMV. Compounds that were positive in the CPE assay were confirmed in a similar assay in 96-well plates according to established laboratory protocols with the compounds added 1 h post infection to identify compounds that inhibit early stages of replication including adsorption and penetration. Additionally, 12 1:3 dilutions were done instead of 6 1:5 dilutions for added accuracy. Genome copy number was determined by qPCR using primers 5′-TCAGCCATCAACTCTGCTACCAAC-3′ (SEQ ID NO: 13), 5′ ATCTGAAACAGCCGTATATCATCTrG-3′ (SEQ ID NO: 14), and probe 5′-TTCTCTGTCAGCTAGCCAATGATATCTTCGAGC-3′ (SEQ ID NO: 15).
vi. Assays for EBV 9 (Epstein Barr), HHV-6B (Human Herpesvirus), and HHV-8 Assays for EBV (Epstein Barr), HHV-6B and HHV-8 were performed by methods previously reported (Keith et al., 2018). For EBV assays, Akata cells were induced to undergo a lytic infection with 50 μg/ml of a goat anti-human IgG antibody. Experimental compounds were diluted within plates; the cells were added and incubated for 72 h. For HHV-6 assays, compounds were serially diluted in plates then uninfected Molt-3 cells were added to each well. Infection was initiated by adding HHV-6B infected Molt-3 cells, at a ratio of approximately 1 infected cell for every 10 uninfected cells. Assay plates were incubated for seven days at 37° C. Assays for HHV-8 were performed in BCBL-1 cells. Similar plates were initiated without virus induction/addition and used for measuring cytotoxicity by the addition of CTG. For all assays, the replication of the virus was assessed by the quantification of viral DNA. For EBV, primers 5′-CCC AGG AGT CCC AGT AGT CA-3′ (SEQ ID NO: 16), and 5′-CAG TTC CTC GCCTTAGGTTG-3 (SEQ ID NO: 17), amplified a fragment corresponding to coordinates 96802-97234 in EBV genome (AJ507799). For HHV-8, 5′-TTC CCC AGA TAC ACG ACA GAA TC-3′ (SEQ ID NO: 18), reverse primer 5′-CGG AGC GCA GGC TAC CT-3′ (SEQ ID NO: 19), and probe 5′-(FAM) CCT ACG TGT TCG TCG AC (TAMRA)-3′ (SEQ ID NO: 20). Plasmid pMP218 containing a DNA sequence corresponding to nucleotides 14120-14182 (AF148805.2) was used to provide absolute quantification of viral DNA. For HHV-6B, 5′-GTT AGG GTA TAC CGA TGT GCG TGA T-3′ (SEQ ID NO: 21), 5′-TAC AGA TAC GGA GGC AAT AGA TTC G-3′ (SEQ ID NO: 22), and 5′-(FAM) TCC GAA ACA ACT GTC TGA CTG GCA AAA-3′ (SEQ ID NO: 23), were used to quantify virus DNA. Compound concentrations sufficient to reduce genome copy number by 50% were calculated from experimental data as well as compound cytotoxicity. Secondary assays are performed similarly, with the addition of 50 μg/ml of goat anti-human-IgG antibody to cells (EBV), initiation of infection by adding infected cells to uninfected cells (HHV-6B) or induction of HHV-8 in BCBL-1 cells—all performed and incubated for 1 h prior to adding the cells to plates containing diluted compounds at 12 three-fold concentrations.
vii. Assays for BK virus and JC virus. Primary assays for BKV and JCV were performed by methods previously reported (Keith et al., 2018). For BKV, compound dilutions were prepared in plates containing cells, subsequently infected and incubated for 7 d. Total DNA was prepared and genome copy number was quantified by real time PCR using the primers 5′-AGT GGA TGG GCA GCC TAT GTA-3′ (SEQ ID NO: 24), 5′-TCA TAT CTG GGT CCC CTG GA-3′ (SEQ ID NO: 25), and probe 5′-6-FAM AGG TAG AAG AGG TA GGG TGT TTG ATG GCA CAG TAMRA-3′ (SEQ ID NO: 26), (Leung et al., 2001). Plasmid pMP526 serves as the DNA standard for quantification purposes. Compounds that were positive in this assay were confirmed in a similar assay in 96-well plates according to established laboratory protocols with the compounds added 1 h post infection to identify compounds that inhibit early stages of replication including adsorption and penetration. Genome copy number was determined by methods described above.
   Primary evaluation of compounds against JC virus were also performed by methods similar to those for BK virus primary assays but were done in COS7 cells and utilized the 1-4 strain of JCV in COS7 cells. Viral DNA was quantified using primers 5′-CTG GTC ATG TGG ATG CTG TCA-3′(SEQ ID NO: 27), and 5′-GCC AGC AGG CTG TTG ATA CTG-3′ (SEQ ID NO: 28), and probe 5′-6-FAM-CCC TIT GTT TGG CTG CT-TAMRA-3 (SEQ ID NO: 29), together with the plasmid pMP508 to provide a standard curve for absolute quantification. Secondary assays against JCV were also performed in COS7 cells by methods similar to those for BK virus to identify compounds that inhibited adsorption or penetration of the virus.
      viii. Assays for HPV (Human papillomavirus)
Primay assay: An HPV11 replicon assay was developed and expresses the essential E1 and E2 proteins from the native promoter. The E2 origin binding protein interacts with the virus origin of replication and recruits the E1 replicative helicase which unwinds the DNA and helps to recruit the cellular DNA replication machinery (including DNA polymerases, type I and type II topoisomerases, DNA ligase, single-stranded DNA binding proteins, proliferating cell nuclear antigen). The replication complex then drives the amplification of the replicon which can be assessed by the expression of a destabilized NanoLuc reporter gene carried on the replicon. In this assay, the replicon (pMP619) is transfected into C-33 A cells grown as monolayers in 384-well plates. At 48 h post transfection, the enzymatic activity of the destabilized NanoLuc reporter is assessed with NanoGlo reagent. The reference compound for this assay is PMEG and its EC50 value is within the prescribed range of 2-9.2 μM and is similar to a compound reported previously (Beadle et al., 2016).
Secondary Assay: HPV genome replication is done by methods similar to HPV 11 but in a plasmid, system utilizing HPV16 and/or HPV18. If only one strain is tested, an additional replicate is tested to ensure that the estimates of EC₅₀ and EC₉₀ values are more precise. Recent publications demonstrate our ability to perform these studies (Beadle et al., 2016).

### Syntheses

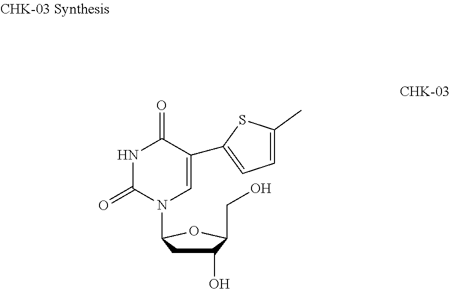

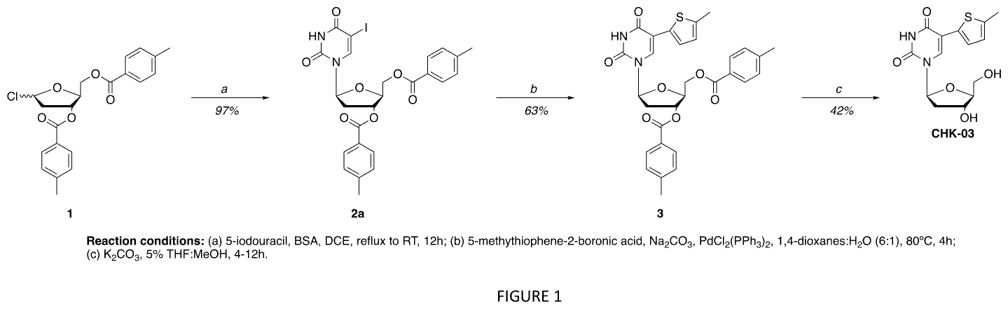

As shown in FIG. 1, to a suspension of 5-iodouracil (3.979 g, 16.72 mmol, 1.3 eq) in dichloroethane, N,O-Bis(trimethylsilyl)acetamide (6.3 mL, 25.73 mmol, 2.0 eq) was added. The reaction mixture was heated to 85° C. and refluxed under nitrogen until a clear solution was obtained (ca. 30 minutes). The reaction mixture was cooled quickly to room temperature using an ice-water bath. Compound 1 (5.002 g, 12.6 mmol, 1.0 eq) was added and stirred at room temperature overnight. The reaction mixture was diluted with ethanol and allowed to stir for 10 minutes. Solvent was removed via rotary evaporation to yield crude product 2a ((2S,3R,5S)-5-(5-iodo-2,4-dioxo-3,4-dihydropyrimidin-1(2H)-yl)-2-(((4-methylbenzoyl)oxy)methyl)tetrahydrofuran-3-yl 4-methylbenzoate) as a yellow syrup. The purified product was obtained via flash chromatography (50% EtOAc:Hexanes) as a white, crystalline solid in 7.364 g (12.47 mmol, 97% yield). TLC Rr: 0.44 (50% EtOAc:Hexanes). ¹H NMR (400 MHz, DMSO-d₆): δ 11.63 (s, 3-NH, 1H), 8.41 (s, H-6, 1H), 7.79 (t, Tol, 4H), 7.22 (t, Tol, 4H), 6.22 (t, H-1′, 1H), 5.51 (m, H-3′, 1H), 4.55 (d, H-5′, 2H), 4.50 (m, H-4′, 1H), 2.64 (m, H-2′, 2H), 2.28 (s, Tol-CH₃, 3H), 2.25 (s, Tol-CH₃, 3H). ¹³C NMR (400 MHz, DMSO-d₆): δ 164.4, 164.1, 161.2, 149.5, 143.8, 143.2, 142.7, 129.5, 125.7, 84.8, 81.1, 72.0, 70.1, 63.9, 36.2, 20.6. MS calcd for C₂₅H₂₃IN₂O₇ [M]⁺: 591.06, found: 591.1.

To a solution of 2a (0.500 g, 0.85 mmol, 1.0 eq) and dichlorobis(triphenylphosphine)Pd(II) (0.091, 0.13 mmol, 0.15 eq) dissolved in a 6:1 solution of dioxanes (30.0 mL) and water (5.0 mL), 5-methylthiophene-2-boronic acid (0.241 g, 1.70 mmol, 2.0 eq) and sodium carbonate (0.269 g, 2.54 mmol, 3.0 eq) were added. The reaction mixture was heated to 80° C. under nitrogen and allowed to stir for 4 h. The reaction mixture was then cooled to rt and filtered over a celite pad. The TLC of the crude reaction mixture showed the product as a vibrant blue spot under UV light. Solvent was removed via rotary evaporation to afford the crude product 3 (((2S,3R,5S)-3-((4-methylbenzoyl)oxy)-5-(5-(5-methylthiophen-2-yl)-2,4-dioxo-3,4-dihydropyrimidin-1(2H)-yl)tetrahydrofuran-2-yl)methyl-4-methylbenzoate) as an amber oil. Compound 3 was then used without further purification in the next step. TLC Rr: 0.68 (10% MeOH:DCM).

To a solution of 3 in 5% THF:MeOH, potassium carbonate (0.296 g, 2.14 mmol, 2.5 eq) was added. The reaction mixture was stirred at room temperature and monitored via TLC (15% MeOH:CHCl₃) until completion. Solvent was removed via rotary evaporation, yielding the crude product as an amber oil. Purification via flash chromatography (15% MeOH:CHCl₃) afforded the purified product CHK-03 (1-((2S,4R,5S)-4-hydroxy-5-(hydroxymethyl)tetrahydrofuran-2-yl)-5-(5-methylthiophen-2-yl)pyrimidine-2,4(1H,3H)-dione) as a pale yellow powder in 0.112 g (0.35 mmol, 42% yield). TLC Rr: 0.48 (15% MeOH:CHCl₃). ¹H NMR (400 MHz, DMSO-d₆): δ 11.61 (s, 3-NH, 1H), 8.42 (s, H-6, 1H), 7.17 (d, H-3″, 1H), 6.73 (d, H4″, 1H), 6.22 (t, H-1′, 1H), 5.30 (d, 3′-OH, 1H), 5.28 (d, 5′-OH, 1H), 4.29 (m, H-3′, 1H), 3.81 (m, H-4′, 1H), 3.65 (br s, H-5′, 2H), 2.38 (s, 5″-Me, 3H), 2.20 (t, H-2′, 2H). ¹³C NMR (400 MHz, DMSO-d₆): δ 162.4, 149.1, 139.3, 134.7, 131.2, 123.6, 122.2, 107.3, 87.4, 84.6, 70.1, 61.6, 13.9. MS calcd for C₂₅H₂₃IN₂O₇ [M+H]⁺: 325.09, found: 325.0. Elemental Analysis calcd for C₁₄H₁₆N₂O₅S+0.05 MeOH+0.45 H₂O: C, 52.07; H, 5.46; N, 7.66. Found: C, 52.34; H, 5.16; N, 7.39.

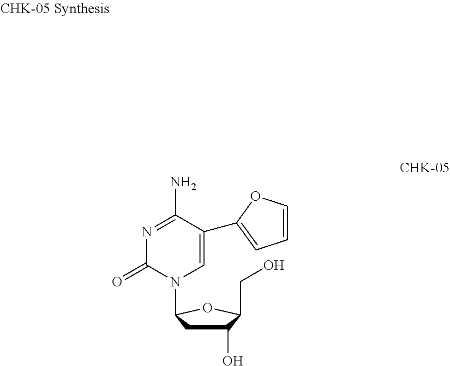

To a suspension of 5-iodocytosine (7.913 g, 33.39 mmol, 1.3 eq) in dichloroethane N,O-Bis(trimethylsilyl)acetamide (12.6 mL, 51.53 mmol, 2.0 eq) was added. The reaction mixture was heated to 85° C. and refluxed under nitrogen until a clear solution was obtained (ca. 30 minutes). The reaction mixture was cooled quickly to room temperature using an ice-water bath. Compound 1 (10.000 g, 25.72 mmol, 1.0 eq) was added and stirred at room temperature overnight. The reaction mixture was diluted with ethanol and allowed to stir for 10 minutes. The solvent was removed via rotary evaporation to yield the crude product 2b ((2S,3R,5S)-5-(4-amino-5-iodo-2-oxopyrimidin-1(2H)-yl)-2-(((4-methylbenzoyl)oxy)methyl)tetrahydrofuran-3-yl 4-methylbenzoate) as an amber syrup. The purified product was obtained via flash chromatography (50% EtOAc:Hexanes) as an off-white, crystalline solid in 13.642 g (23.15 mmol, 90% yield). TLC Rr: 0.32 (50% EtOAc:Hexanes). ¹H NMR (400 MHz, DMSO-d₆): δ 8.98 (s, H-6, 1H), 7.81 (t, Tol, 4H), 7.61 (br s, 4-NH₂, 1H), 7.21 (t, Tol, 4H), 6.68 (br s, 4-NH₂, 1H), 6.17 (t, H-1′, 1H), 5.76 (m, H-3′, 1H), 4.49 (d, H-5′, 2H), 4.04 (m, H-4′, 1H), 2.49 (m, H-2′, 2H), 2.26 (s, Tol-CH₃, 3H), 2.22 (s, Tol-CH₃, 3H). ¹³C NMR (400 MHz, DMSO-d₆): δ 164.8, 164.6, 161.6, 154.8, 149.3, 142.9, 142.6, 130.3, 126.4, 85.3, 82.8, 72.7, 70.5, 62.7, 38.1, 20.8. MS calcd for C₂₅H₂₃IN₂O₇ [M+H]⁺: 590.08, found: 589.9.

Furan-2-boronic acid (0.286 g, 2.55 mmol, 2.0 eq) and sodium carbonate (3.80 mmol, 3.0 eq) were added to a solution of 2b (0.751 g, 1.27 mmol, 1.0 eq) and dichlorobis(triphenylphosphine)Pd(II) (0.088 g, 0.13 mmol, 0.1 eq) dissolved in a 6:1 solution of dioxanes (30.0 mL) and water (5.0 mL). The reaction mixture was heated to 80° C. under nitrogen and allowed to stir for 4 h. The reaction mixture was then cooled to room temperature and filtered over a celite pad. The TLC of the crude reaction mixture showed the product as a vibrant blue spot under UV light. The solvent was removed via rotary evaporation to afford the crude product 4 ((2S,3R,5S)-5-(4-amino-5-(furan-2-yl)-2-oxopyrimidin-1(2H)-yl)-2-(((4-methylbenzoyl)oxy)methyl)tetrahydrofuran-3-yl 4-methylbenzoate) as an amber oil and used without further purification in the next step. TLC Rr: 0.40 (10% MeOH:DCM).

To a solution of 4 in 5% THF:MeOH potassium carbonate (0.442 g, 3.20 mmol, 2.5 eq) was added. The reaction mixture was stirred at room temperature and monitored via TLC (15% MeOH:CHCl₃) until completion. Solvent was removed via rotary evaporation, yielding the crude product as an amber oil. Purification via flash chromatography (15% MeOH:CHCl₃) followed by preparative TLC (15% MeOH:CHCl₃) afforded the purified product CHK-05 (4-amino-5-(furan-2-yl)-1-((2S,4R,5S)-4-hydroxy-5-(hydroxymethyl)tetrahydrofuran-2-yl)pyrimidin-2(1H)-one) as a crystalline white solid in 0.172 g (0.59 mmol, 47% yield). TLC Rr: 0.35 (15% MeOH:CHCl₃). ¹H NMR (400 MHz, DMSO-d): S 8.22 (s, H-6, 1H), 7.68 (m, H-5″, 1H), 7.63 (br s, 4-NH₂, 1H), 6.65 (br s, 4NH₂, 1H), 6.54 (m, H-4″+H-3″, 2H), 6.13 (t, H-1′, 1H), 5.18 (t, 3′-OH, 1H), 5.04 (t, 5′-OH, 1H), 4.16 (m, H-3′, 1H), 3.77 (m, H-4′, 1H), 3.54 (m, H-5′, 2H), 2.15 (m, H-2′, 1H), 2.02 (m, H-2′, 1H). ¹³C NMR (400 MHz, DMSO-d₆): δ 162.6, 154.2, 147.6, 142.8, 140.6, 111.8, 107.5, 98.0, 87.7, 86.0, 70.7, 61.9, 39.8. MS calcd for C₂₅H₂₃IN₂O₇ [M+H]⁺: 294.11, found: 294.0. Elemental Analysis calcd for C₁₃H₁₅N₃O₅+0.2 MeOH+0.45 H₂O: C, 51.51; H, 5.47; N, 13.65. Found: C, 51.44; H, 5.27; N, 13.44.

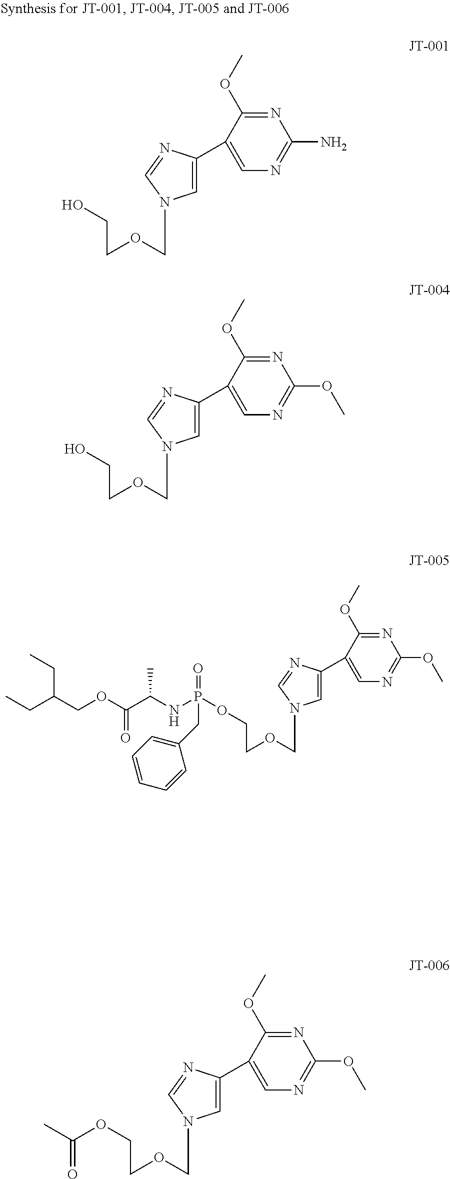

### JT-Int1

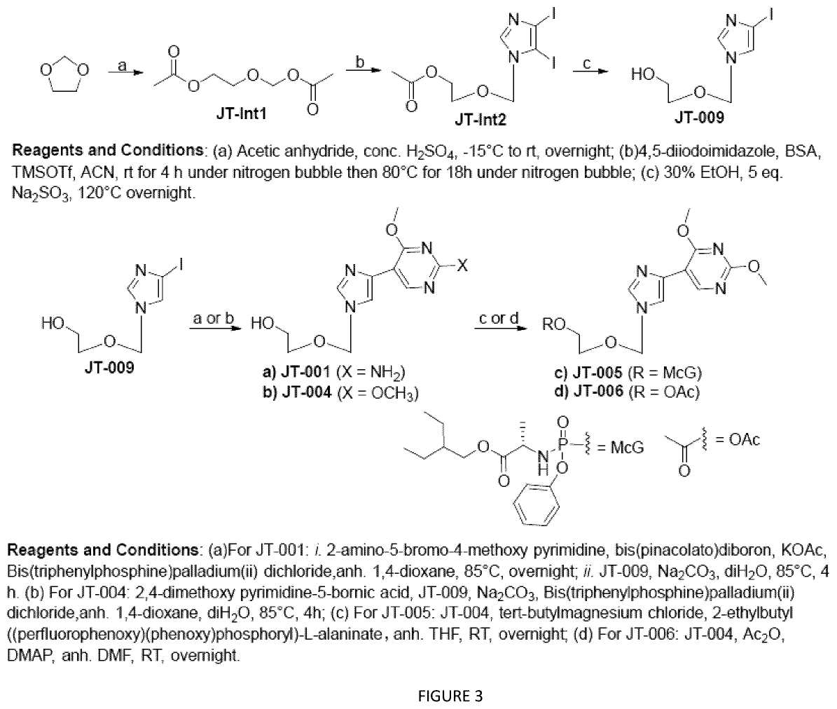

As shown in FIG. 3, to a stirred and cooled (−5° C.) solution of 1,3-dioxolane (23.5 mL, 35.0 mmol) and acetic anhydride (32.2 mL, 35 mmol), conc. H₂SO₄ (98%, d=1.84 g‘cm’, 0.2 mmol) was added dropwise. The reaction mixture was stirred at r.t. for 12 h, then sat. aq NaHCO₃ (10 mL) was added, and the mixture was extracted with CHCl₃ (2×15 mL). The organic extract was dried over anhydrous Na₂SO₄, CHCl₃ was evaporated, and the residue was distilled under reduced pressure to give a clear liquid. ¹H NMR (300 MHz, CDCl₃): δ=2.08, 2.10 (2×s, 6H, CH₃), 3.84-3.87 (m, 2H, CH₂), 4.21-4.24 (m, 2H, CH₂), 5.29 (s, 2H, CH₂). ¹³C NMR (75 MHz, CDCl₃): δ=20.4, 20.6, 62.9, 67.8, 88.7, 170.0, 170.4.

### JT-Int2

4,5-diiodoimidazole (10.0 g, 31.25 mmol) was suspended in anhydrous acetonitrile (200 mL) under nitrogen and stirred at room temperature for 10 mins. Then bis(trimethylsilyl)acetamide (46.0 mL, 187.68 mmol) and JT-Intl (6.0 mL, 37.46 mmol) were added, and the solution was allowed to stir at room temperature under nitrogen for 4 hours. The reaction was cooled to 0° C. and trimethylsilyl trifluoromethanesulfonate (8.2 mL, 47.22 mmol) was then added slowly dropwise over 5 minutes. The reaction was then heated to 80° C. under direct nitrogen for 18 hours. After cooling to room temperature, the reaction was cooled further in a salt/ice bath to 0° C. and quenched with NaHCO₃ (100 mL). After stirring for 10 minutes, the reaction was extracted 5× with CH₂Cl₂ (200 mL). The combined organic layers were dried with MgSO₄, gravity filtered, and the solvent was removed in vacuo to afford an amber oil with yellow solid. The crude mixture was then purified by flash column chromatography on silica gel (40-80% EtoAc in Hexanes) to give the pure product as a white solid with yellow oil (9.5 g, 21.78 mmol, 70%); R_{f}=0.39 (1:1 EtOAc/Hexanes); ¹H NMR (400 MHz, CDCl₃) δ 7.61 (s, 1H), 5.18 (s, 1H), 3.93-3.95 (t, J=4.6 Hz, 2H), 3.45-3.47 (t, J=4.6 Hz, 2H), 1.82 (s, 3H); ¹³C NMR (126 MHz, CDCl₃) δ 170.67, 142.06, 97.32, 82.71, 77.93, 66.69, 62.76, 21.07; MS (ESI+) m/z calc for C₈H₁₀I₂N₂O₃ [M+H⁺]: 436.99, found: 436.9.

### JT-009

To a solution of JT-Int2 (5.6 g, 12.87 mmol) in 30% EtOH and H₂O (125 mL total volume) was added Na₂SO₃ (8.17 g, 64.82 mmol). The solution was then refluxed at 120° C. for 18 hours. After cooling to room temperature, the reaction was concentrated in vacuo to remove the EtOH and was extracted 4× with CH₂Cl₂. The combined organic layers were dried with MgSO₄ and gravity filtered. The solvent was removed in vacuo to afford a white solid. The crude product was then purified by flash column chromatography on silica gel (0-5% CH₃OH in CH₂Cl₂) to give the pure product as a white solid (2.73 g, 10.30 mmol, 79%); R_{f}=0.79 (1:3 CH₃OH/CH₂Cl₂); ¹H NMR (400 MHz, DMSO-d₆) δ 7.74 (s, 1H), 7.46 (s, 1H), 5.31 (s, 2H), 4.65-4.68 (t, J=10.5 Hz, 1H), 3.42-3.47 (m, 2H), 3.35-3.37 (m, 2H); ¹³C NMR (126 MHz, DMSO-d₆) δ 140.5, 125.5, 83.4, 76.2, 70.7, 60.3; MS (ESI+) m/z calcd for C₆H₉IN₂O₂ [M+H⁺]: 269.05, found 269.0.

### JT-001

To a dry round bottom flask under nitrogen, 2-amino-5-bromo-4-methoxy pyrimidine (0.50 g, 2.45 mmol), bis(pinacolato)diboron (0.75 g, 2.94 mmol), and potassium acetate (0.72 g, 7.35 mmol), were added and suspended in 1,4-dioxane (30 mL). Bis(triphenylphosphine)palladium(ii) dichloride (0.172 g, 0.25 mmol) was added to the reaction flask. The reaction was then heated to 85° C. overnight with stirring. The sugar moiety, JT-009 (0.65 g, 2.45 mmol), Na₂CO₃ (0.52 g, 4.90 mmol), and 5 mL of diH₂O were added. The reaction was heated to 85° C. for 4 hours. After stirring, the reaction was cooled to room temperature and the solvent was removed in vacuo. The residue was suspended in CH₂Cl₂/EtOAc and Na₂CO₃ was gravity filtered away from the solution. Solvent was removed in vacuo to give the crude product as an orange oil. The crude product was purified via flash column chromatography (0-10% CH₃OH in CH₂Cl₂) to afford a yellow oil. The residue was then washed with minimal aqueous sodium thiosulfate and extracted with EtOAc 3-4×. The organic layer was dried with MgSO₄, gravity filtered, and the solvent was removed in vacuo to give a pink solid. The product was further purified via flash column chromatography on silica gel (5-10% CH₃OH in CH₂Cl₂) to afford the product as a light pink solid (0.19 g, 0.72 mmol, 30%); R_{f}=0.26 (1:9 CH₃OH/CH₂Cl₂); ¹H NMR (400 MHz, DMSO-d₆) δ 8.59 (s, 1H), 7.77 (s, 1H), 7.37 (s, 1H), 6.52 (s, 2H), 5.34 (s, 2H), 4.64-4.66 (t, J=11.0 Hz, 1H), 3.91 (s, 3H), 3.41-3.44 (t, J=10.0 Hz, 2H), 3.36-3.38 (t, J=9.6 Hz, 2H); ¹³C NMR (126 MHz, DMSO-d₆) δ 165.78, 162.24, 155.48, 138.08, 134.72, 117.18, 104.65, 76.21, 70.39, 60.44, 53.67; MS (ESI+) m/z calcd for C₁₁H₁₅N₅O₃ [M+H⁺]: 266.27, found 266.2; Elemental analysis: Anal. Calcd for C₁₁H₁₅N₅O₃: C, 49.81; H, 5.70; N, 26.40. Found: C, 49.74; H, 5.76; N, 26.12.

### JT-004

To a dry round bottom flask under nitrogen, 2,4-dimethoxy pyrimidine-5-bornic acid (0.69 g, 3.73 mmol) was suspended in anhydrous 1,4-dioxane and flushed with nitrogen for 15 minutes while stirring. JT-009 (0.5 g, 1.87 mmol), Na₂CO₃ (0.39 g, 3.73 mmol), and Bis(triphenylphosphine)palladium(ii) dichloride (0.131 g, 0.18 mmol) were added to the reaction along with 5 mL of diH₂O. The reaction was heated to 85° C. for 4 hours. After stirring, the reaction was cooled to room temperature and the solvent was removed in vacuo. The residue was suspended in CH₂Cl₂/CH₃OH and Na₂CO₃ was gravity filtered away from the solution. Solvent was removed in vacuo to give the crude product as a yellow solid with a yellow oil. The crude product was purified via flash chromatography on silica gel (0-5% CH₃OH in CH₂Cl₂) to afford the product as a white solid (0.31 g, 1.11 mmol, 48%); R_{f}=0.38 (1:9 CH₃OH/CH₂Cl₂); ¹H NMR (400 MHz, CD₃OD) δ 8.77 (s, 1H), 7.87 (s, 1H), 7.63 (s, 1H), 5.44 (s, 2H), 4.10 (s, 3H), 3.98 (s, 3H), 3.62-3.64 (t, J=9.1 Hz, 2H), 3.51-3.64 (t, J=9.6 Hz, 2H); ¹³C NMR (126 MHz, CD₃OD) δ 167.13, 163.70, 154.60, 137.89, 133.13, 118.77, 108.89, 76.54, 70.07, 60.57, 54.08, 53.48; MS (ESI+) m/z calcd for C₁₂H₁₆N₄O₄ [M+H⁺]: 281.28, found: 281.2; Elemental analysis: Anal. calcd for C₁₂H₁₆N₄O₄: C, 51.42; H, 5.75; N, 19.99. Found: C, 50.99; H, 5.68; N, 19.72.

### JT-005

To a solution of JT-004 (0.042 g, 0.15 mmol) in anh. THF (3 mL) under a nitrogen atmosphere was added tertbutylmagnesium chloride (1M in THF, 0.30 mL, 0.30 mmol) dropwise, and the reaction was stirred at room temperature for 30 minutes. In a separate flask, 2-ethylbutyl ((perfluorophenoxy)(phenoxy)phosphoryl)-L-alaninate was suspended in anh. THF (4 mL) and added to the reaction vessel. The reaction was allowed to stir at room temperature under a nitrogen atmosphere overnight. After stirring overnight, the reaction was quenched with CH₃OH (2 mL) and stirred for another 5 minutes before removal of solvent in vacuo. The crude mixture was then purified twice by flash column chromatography on silica gel (0-10% CH₃OH in CH₂Cl₂) to give the product as a yellow oil (0.075 g, 0.13 mmol, 86% as a mix of diastereomers); R_{f}=0.48 (1:9 CH₃OH/CH₂Cl₂); ¹H NMR (400 MHz, CDCl₃) δ 8.96 (s, 1H), 7.58-7.60 (d, J=5.9 Hz, 1H), 7.44-7.45 (d, J=2.7 Hz, 1H), 7.23-7.27 (m, 2H), 7.07-7.16 (m, 3H), 5.26-5.28 (app. d, 2H), 4.13-4.21 (m, 2H), 3.92-4.05 (m, 8H), 3.72-3.82 (m, 1H), 3.58-3.63 (m, 2H), 1.41-1.59 (m, 1H), 1.24-1.34 (m, 8H), 0.79-0.84 (m, 6H); ¹³C NMR (126 MHz, CDCl₃) δ 173.65, 166.94, 163.72, 155.64, 150.67, 137.30, 134.72, 129.70, 125.03, 120.20, 118.06, 109.32, 67.53, 65.66, 54.9, 54.16, 50.28, 40.25, 23.21, 21.16, 11.03; MS (ESI+) m/z calcd for C₂₇H₃₈N₅O₈P [M+H⁺]: 592.60, found: 592.3; Elemental Analysis: Anal. calcd for C₂₇H₃₈N₅O₈P+1% CH₃OH: C, 53.93; H, 6.79; N, 11.23. Found: C, 53.89; H, 6.55; N, 11.02.

### JT-006

JT-004 (0.050 g, 0.18 mmol) was suspended in anh. DMF (2 mL) and treated with acetic anhydride (0.051 mL, 0.53 mmol) and dimethylaminopyridine (0.0022 g, 0.018 mmol). The reaction was stirred at room temperature for 3 hours. After stirring, the solvent was removed in vacuo to give the crude product as an orange oil. The crude product was purified via flash chromatography on silica gel (0-5% CH₃OH in CH₂Cl₂) and placed on a vacuum pump overnight to afford the product as a peach colored solid (0.076 g, 0.24 mmol, 86%); R_{f}=0.41 (1:9 CH₃OH/CH₂Cl₂); ¹H NMR (400 MHz, CD₃OD) δ 8.77 (s, 1H), 7.86 (s, 1H), 7.60 (s, 1H), 5.42 (s, 2H), 4.13-4.16 (t, J=9.2 Hz, 2H), 4.09 (s, 3H), 3.97 (s, 3H), 3.66-3.68 (t, J=9.6 Hz, 2H), 1.96 (s, 3H); ¹³C NMR (126 MHz, CD₃OD) δ 171.23, 167.09, 163.70, 154.62, 137.89, 133.19, 118.70, 108.81, 76.32, 66.62, 62.92, 54.11, 53.50, 19.35; MS (ESI+) m/z calcd for C₁₄H₁₈N₄O₅ [M+H⁺]: 323.32, found: 323.2; Elemental analysis: Anal. calcd for C₁₄H₁₈N₄O₅+0.4% H₂O: C, 51.18; H, 5.79; N, 16.93. Found: C, 51.03; H, 5.75; N, 17.00.

### Synthesis of CDW3-002

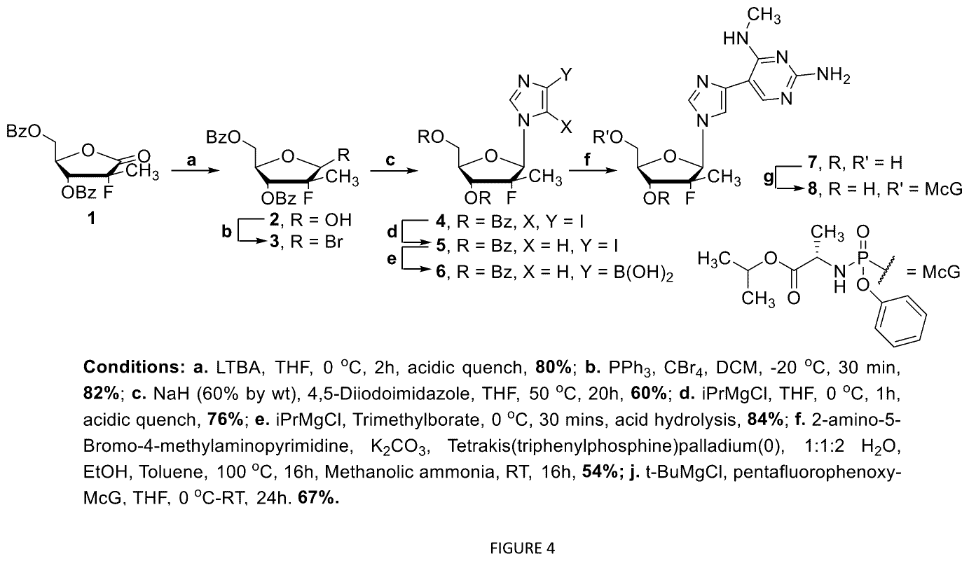

As shown in FIG. 4, to a dry 500 mL three neck round-bottom flask with stir bar and thermometer were added the lactone 1 (20 g, 53.7 mmol, 1 equiv.) and anhydrous THF (200 mL). The solution was cooled to 0° C. under a nitrogen atmosphere, and then was added a solution of lithium tri-tert-butoxyaluminohydride (1.0 M in THF, 64.5 mL, 64.5 mmol 1.2 equiv.), slowly via syringe. After completion of the addition, the reaction mixture was allowed slowly to warm to 25° C. over and stirred for 2 hours. The reaction mixture had the solvent removed under vacuum and the residue was resuspended in 200 mL of ethyl acetate, followed by 200 mL 3M HCl. The organic later was collected and the aqueous layer was extracted from with ethyl acetate (2×100 mL) and the organics were combined. Combined organic layer was washed with 200 mL of water, 200 mL saturated aq NaHCO₃, and 200 mL Brine. The organic layer was dried over MgSO₄, filtered and concentrated under reduced pressure to give a light-yellow syrup. The residue was purified by flash chromatography using 0-25% ethyl acetate/hexanes gradient to give lactol 2 as colorless syrup. The syrup was heated at 50° C. with sonication until a solid is formed to yield pure lactol 2 (((2R,3R,4R,5R)-3-(Benzoyloxy)-4-fluoro-5-hydroxy-4-methyltetrahydrofuran-2-yl)methyl Benzoate) as a white solid (16 g, 42.96 mmol, 80%). mp 79-80° C.; 1H NMR (DMSO-d6, 400 MHz) d 7.99 (m, 2H), 7.93 (m, 2H), 7.70 (m, 1H), 7.61 (m, 1H), 7.55 (m, 2H), 7.42 (m, 2H), 7.32 (d, J=5.2 Hz, 1H), 5.54 (dd, J=23.6, 7.2 Hz, 1H), 5.20 (dd, J=10.8, 5.2 Hz, 1H), 4.55-4.50 (m, 1H), 4.46-4.40 (m, 2H), 1.42 (d, J=22.4 Hz, 3H). 13C NMR (DMSO-d6, 100 MHz) d 166.4, 166.2, 134.9, 134.3, 130.5, 130.3, 130.2, 129.8, 129.6, 129.6, 101.2, 101.1, 77.1, 75.7, 65.9, 17.6.

To a stirred solution of PPh₃ (26.3 g, 100.2 mmol 2.5 equiv.) and β-lactol 2 (15 g, 40.06 mmol, 1 equiv.) in 200 mL DCM, kept below −20° C. under N₂ atmosphere, was added CBr₄ (26.6 g, 80.14 mmol, 2 equiv.) in gram additions, being sure to keep the temperature around −20° C. After completion of the addition, the reaction mixture was stirred at −20° C. for 30 min. Silica gel (25 g) was added to the mixture, filtered through a pad of silica gel (75 g) and washed with cold dichloromethane. The combined filtrates were concentrated under reduced pressure at room temperature to give colorless oil. The oil was purified by flash chromatography using 0-20% ethyl acetate/hexanes gradient to give 3 (((2R,3R,4R,5R)-3-(benzoyloxy)-5-bromo-4-fluoro-4-methyltetrahydrofuran-2-yl)methylbenzoate) as colorless oil which solidified upon standing to give a white solid. (14.36 g, 32.85 mmol, 82%). 1H NMR (CDCl₃, 400 MHz) d 8.13 (d, J=7.2 Hz, 2H), 8.02 (d, J=7.6 Hz, 2H), 7.63-7.56 (m, 2H), 7.50-7.42 (m, 4H), 6.34 (s, 1H), 5.29 (dd, J=5.3, 2.8 Hz, 1H), 4.89-4.86 (m, 1H), 4.80-4.76 (m, 1H), 4.65-4.61 (m, 1H), 1.72 (d, J=21.2 Hz, 3H). 13C NMR (CDCl₃, 100 MHz) d 165.9, 165.7, 133.7, 133.4, 130.0, 129.6, 129.2, 128.8, 128.5, 128.5, 94.6, 92.0, 81.9, 73.3, 62.4, 22.8.

To a dry 500 mL 3-neck round bottom flask with a thermometer, reflux condenser, and magnetic stir bar was added 4,5-Diiodo-(1H)-imidazole (13 g, 40.6 mmol, 1.2 equiv.) and anhydrous THF (200 mL), under a nitrogen atmosphere. The solution was cooled to 0° C. and NaH was added in 3 small portions (1.62 g, 40.6 mmol, 1.2 equiv.). The reaction vessel was allowed to stir and come up to room temperature for 30 minutes. In a separate round bottom flask containing 3 (14.8 g, 33.8 mmol, 1 equiv.) under a nitrogen atmosphere is added anhydrous THF (50 mL). 3 is dissolved with mild agitation and added to the three-neck round bottom flask via a cannula with nitrogen back pressure. The resulting solution is then heated to reflux for 18 hours, over which the solution will take on a dark brown color and a solid white precipitate will crash out. Reaction was cooled to room temperature and quenched with 50 mL of sat. aq. NH₄Cl and extracted three times with 100 mL of ethyl acetate. Combined organics were washed with brine, dried over MgSO₄, filtered and rotary evaporated to dryness. Crude sample was loaded onto silica and columned via Combi-flash chromatography system from 0-30% ethyl acetate/hexanes. Solvents were removed to afford 4 (((2R,3R,4R,5R)-3-(benzoyloxy)-5-(4,5-diiodo-1H-imidazol-1-yl)-4-fluoro-4-methyloxolan-2-yl)methyl benzoate) as a clear oil with a slightly yellow color. Resuspending in minimal DCM, adding hexanes until solution turns cloudy, and removing solvent under vacuum yields a off-white solid, 13.7 g, 20.28 mmol, 60% yield. R_{f}=0.5 in 25% ethyl acetate/hexanes. 1H NMR (400 MHz, DMSO-D6), 8.16 (1H, s), 8.0 (2H, d), 7.9 (2H, d), 7.69 (2H, dt), 7.61 (2H, dt), 7.53 (1H, t), 7.43 (1H, t), 6.05 (1H, d), 5.63 (1H, dd), 4.69 (3H, m), 1.17 (3H, d). 13C NMR (100 MHz, DMSO-D6) 165.6, 165.3, 141.1, 134.6, 134.1, 130.2, 129.7, 129.6, 129.5, 129.3, 128.9, 101.1, 99.3, 98.8, 94.1, 93.7, 85.4, 77.1, 73.7, 73.5, 63.9, 18.8, 18.5.

To a dry 250 mL round bottom flask compound 4 (1 g, 1.47 mmol, 1 equiv.) and dry THF (50 mL) was added under a nitrogen atmosphere. The solution was cooled to −20° C. in a 3:1 w/w Ice:Salt bath for 10 mins before slow addition of isopropylmagnesium chloride solution (0.75 mL, 2M in THF, 1.5 mmol, ˜1 equiv.), continually maintaining the temperature below −10° C. The reaction mixture was then stirred at −20° C. for 30 minutes before quenching with sat. aq. NH₄Cl at −20° C. The reaction mixture was allowed to come to room temperature and the aqueous layer was extracted 3× with ethyl acetate. The organic layers were washed with brine, dried over MgSO₄, filtered and rotary evaporated to dryness. The residue was loaded onto silica and columned via Combi-flash chromatography eluting with 0-50% ethyl acetate/hexanes. The solvents were removed via rotary evaporation to afford 5 (((2R,3R,4R,5R)-3-(benzoyloxy)-4-fluoro-5-(4-iodo-1H-imidazol-1-yl)-4-methyloxolan-2-yl)methyl benzoate) as a white solid (614 mg, 1.12 mmol, 76% yield). R_{f}=0.5 in 50% ethyl acetate/hexanes. 1H NMR (400 MHz, CDCl₃), 8.09 (4H, ddd), 7.63 (3H, m), 7.51 (4H, dt), 7.24 (1H, d), 5.90 (1H, d), 5.63 (1H, dd), 4.87 (1H dd), 4.69 (1H, m), 4.60 (1H, dd), 1.25 (3H, d). ¹³C NMR (100 MHz, DMSO-D6) 166, 165.4, 139.6, 134.1, 130.2, 129.7, 129.43, 129.4, 129, 122.9, 101.5, 99.8, 90.9, 90.5, 77.2, 73.5, 73.3, 63.7, 31, 17.5, 17.3.

To a dry 100 mL round bottom flask, 5 (1 g, 1.82 mmol) and 50 mL of dry THF under nitrogen. The flask was cooled to −20° C. in a 3:1 w/w ice/salt water bath for 10 mins before the slow addition of isopropylmagnesium chloride solution (0.91 mL, 2M in THF, 1.82 mmol, 1 equiv.). The reaction was stirred for 15 mins at −20° C. before the addition of trimethyl borate (1 mL, 9.1 mmol, 5 equiv.). The reaction was stirred for 15 mins at −20° C. before warming to room temperature, then stirred for another 15 mins. The reaction mixture was treated with 10 mL of 1M HCl (aq) and the solution left to stir for 20 minutes. The solution was poured into 100 mL of sat. aq NaHCO₃ and extracted with 50 mL of ethyl acetate (3×). The organic layers were combined, washed with brine (100 mL) and dried over MgSO₄, filtered, and the solvent was removed under vacuum. Coevaporation with DCM/Hexanes afforded a white foam 6 ((1-((2R,3R,4R,5R)-4-(benzoyloxy)-5-((benzoyloxy)methyl)-3-fluoro-3-methyloxolan-2-yl)-1H-imidazol-4-yl)boronic acid) that was used in the next step without further purification. 724.4 mg, 1.56 mmol, 85% yield. (ESI+) m/z calcd for C₂₃H₂₂BFN₂O₇ [M+H]: 469.24, found: 468.95.

In a Schlenk flask with stir bar 10 mL of 1:1:2 H₂O/ethanol/toluene was added. The solvent was then degassed using a freeze-pump-thaw cycle three times. The flask was then flushed with argon and compound 6 (725 mg, 1.6 mmol, 1.6 equiv.), 2-amino-5-Bromo-4-methylaminopyrimidine (204 mg, 1 mmol, 1 equiv.), K₂CO₃ (415 mg, 3 mmol, 3 equiv.) and tetrakis(triphenylphosphine)palladium(0) (116 mg, 1 mmol, 1 equiv.) were added. The flask was capped and briefly put under vacuum and then flushed with argon before sealing. The flask was heated at 100° C. for 16 hours, and then cooled to room temperature. The flask was unsealed, the solution transferred to a round bottom flask, and the solvents removed under vacuum. The residue was resuspended in ethyl acetate and washed with saturated aqueous NaHCO₃. The organic layer was filtered over celite and the solvent removed under vacuum. The residue was resuspended in minimal methanol and transferred to a clean Schlenk flask, followed by addition of 15 mL of 7M NH₃ in methanol. The reaction was then stirred at room temperature for 16 hours. The flask was sparged with N₂ to remove NH₃ and the methanol removed under vacuum. The residue was then loaded onto silica and purified by flash chromatography in 8:1:1:0.5 ethyl acetate/acetone/methanol/H₂O. Fractions containing peaks of interest were loaded onto C18 Silica and purified by flash chromatography eluting with 2.5-100% methanol/H₂O. The methanol was removed under vacuum and the residue diluted with H₂O and lyophilized to give a white fluffy solid CDW3-001 ((2R,3R,4R,5R)-5-(4-(2-amino-4-(methylamino)pyrimidin-5-yl)-1H-imidazol-1-yl)-4-fluoro-2-(hydroxymethyl)-4-methyloxolan-3-ol) (169 mg, 0.54 mmol, 54% yield). 1HNMR (400 MHz, DMSO-D6) 8.42 (1H, m), 8.04 (1H, s), 7.97 (1H, s), 5.99 (2H, s), 5.91 (1H, d), 5.59 (1H, s), 5.18 (1H, t), 4.05 (1H, dot), 3.83 (1H, m), 3.64 (1H, m), 2.86 (3H, d), 1.0 (3H, d) (ESI+) m/z calcd for C14H19FN6O3 [M+H]: 339.15, found: 339.13 Elemental analysis: Anal. calcd for C₁₄H₁₉FN₆O₃: C, 49.7; H, 5.66; N, 24.8. Found: C, 49.2; H, 5.68; N, 24.6.

To a dry 100 mL flask, 7 (50 mg, 0.14 mmol) and dry THF (20 mL) were added. The flask was cooled to 0° C. and 1M t-BuMgCl in THF (0.28 mL, 0.28 mmol, 2 equiv.) was added dropwise. The reaction was warmed to room temperature and stirred for 15 min, at which point (S)-2-[(S)-(2,3,4,5,6-pentafluorophenoxy)phenoxyphosphorylamino]propionic acid isopropyl ester (76 mg, 0.17 mmol, 1.2 equiv) dissolved in 5 mL of dry THF was added and the reaction stirred for 24 hours. The reaction was quenched with 10 mL sat aq. NaHCO₃, and diluted with 60 mL ethyl acetate and 20 mL H₂O. The organic layer was separated, washed with brine (100 mL), dried over MgSO₄, and filtered. Solvent was removed under vacuum and the sample was loaded onto silica with purification by flash chromatography in 0-10% methanol/DCM. Solvent was removed under vacuum to yield a white fluffy solid 8 (CDW3-002-propan-2-yl-(2S)-2-(((((2R,3R,4R,5R)-5-(4-(2-amino-4-(methylamino)pyrimidin-5-yl)-1H-imidazol-1-yl)-4-fluoro-3-hydroxy-4-methyloxolan-2-yl)methyl)(phenyl)phosphono)amino) propanoate) (57 mg, 0.094 mmol, 67% yield). 1H NMR (400 MHz, DMSO-D6): 8.52 (1H, s, broad), 8.1 (1H, s), 7.96 (1H, d), 7.57 (1H, s), 7.34-7.11 (6H, m), 6.11 (2H, s, broad), 6.04-5.96 (2H, m), 5.83 (1H, d), 4.75 (1H, p), 4.3 (2H, dodod), 4.08-4.02 (2H, m), 3.74 (1H, m), 2.86 (3H, d), 1.16 (3H, d), 1.07-1.04 (9H, m). (ESI+) m/z calcd for C₂₆H₃SFN₇O₇P [M+H]: 608.24, found: 608.1 Elemental analysis: Anal. calcd for C₂₆H₃₅FN₇O₇P: C, 51.4; H, 5.81; N, 16.14. Found: C, 51.2; H, 5.8; N, 15.95.

The results of the effectiveness of specific structures relating to different viruses are shown in Tables 1 to 7 set forth below. When reviewing the results, it should be noted that the lower the EC₅₀, the less the concentration of a drug is required to produce 50% of maximum effect and the higher the potency.

Table 1 relates to the use of the following structures to treat Epstein-Barr Virus. Reviewing the EC₅₀ values, the control showed an EC₅₀ value of 3.92. However, CHK-03 showed an EC₅₀ value of 2.97. Other relevant molecules showed the following values of JT-001 of 59.87; JT-004 of 86.77 and JT-006 of 79.91.

Table 2 relates to the use of the following structure to treat Epstein-Barr Virus. Reviewing the EC₅₀ values, the control showed an EC₅₀ value of 5.49. Notably the CHK-05 structure showed an EC₅₀ value of 69.85.

Table 3 relates to the use of the following structure to treat MERS Coronavirus. Reviewing the EC₅₀ values, the control showed an EC₅₀ value of 0.036. Notably the CHK-05 molecule showed an EC₅₀ value of >45. Notably this molecule also showed an SI value>10.

Table 4 relates to the use of the following structure to treat Enterovirus. Reviewing the EC₅ values, the control showed an EC₅₀ value of 0.12. Notably the CHK-05 molecule showed an EC₅₀ value of 24 and >36.

Table 5 relates to the use of the following structure to treat Chikungunya virus. Reviewing the EC₅₀ values, the control showed an EC₅₀ value of 3.2 and 7.9 respectively depending on the assay. Notably the CHK-05 molecule showed an EC₅₀ value of >68 and >42 again, depending on the assay.

Table 6 relates to the use of the following structures to treat Ebola virus. Reviewing the EC₅₀ values, the control showed an EC₅₀ value of 12. The CDW3-002 molecule showed an EC₅₀ of 30. The CHK-05 showed an EC₅₀ of 18 and the JT-005 molecule showed an EC₅₀ value of 12. Notably the CHK-05 molecule showed an EC₅₀ value of >68 and >42 again depending on the assay.

Table 7 relates to the use of the following structure to treat Dengue Virus. Reviewing the EC₅₀ values, the control showed an EC₅₀ value of 0.36 and 0.43 depending on the assays used. Notably the CHK-05 molecule showed an EC₅₀ value of 79.

TABLE 1
In Vitro Antiviral Screening Results (Epstein Barr Virus)

| | |
|---|---|
| Virus screened: | Epstein-Barr virus |
| Virus strain: | Akata |
| Cell line: | Akata |
| Vehicle: | DMSO |
| Drug conc. Range: | 0.032-100 μM |
| Control conc. Range: | 0.032-100 μM |
| Experiment number: | 22-ebv-011 |

| Control | Control | | | | | | |
|---|---|---|---|---|---|---|---|
| drug | assay | | | | | | |
| name | order | Control assay name | EC₅₀ | EC₉₀ | CC₅₀ | SI₅₀ | SI₉₀ |
| Cidofovir | Primary | Quantitative polymerase chain | 3.92 | 71.40 | >100.00 | >26 | >1 |
| | | reaction (DNA)/CellTiter-Glo | | | | | |
| | | (Toxicity) | | | | | |

EC₅₀—compound concentration that reduces viral replication by 50%
SI₅₀—CC₅₀/EC₅₀
EC₉₀—compound concentration that reduces viral replication by 90%
SI₉₀—CC₅₀/EC₉₀
CC₅₀—compound concentration that reduces cell viability by 50%

Screening Results

| | Date | | | | | | | | |
|---|---|---|---|---|---|---|---|---|---|
| Product | Received | Compound | Drug | | | | | | |
| number | (m/dd/yy) | name/ID | assay order | Drug assay name | EC₅₀ | EC₉₀ | CC₅₀ | SI₅₀ | SI₉₀ |
| C-2022- | Jun. 8, 2022 | CHK-03 | Primary | Quantitative | 2.97 | 18.74 | >100.00 | >34 | >5 |
| 60239 | | | | polymerase chain | | | | | |
| | | | | reaction | | | | | |
| | | | | (DNA)/CellTiter-Glo | | | | | |
| | | | | (Toxicity) | | | | | |
| C-2022- | Jun. 8, 2022 | JT-001 | Primary | Quantitative | 59.87 | >100.00 | \|>100.00 | >2 | 1 |
| 60266 | | | | polymerase chain | | | | | |
| | | | | reaction | | | | | |
| | | | | (DNA)/CellTiter-Glo | | | | | |
| | | | | (Toxicity) | | | | | |
| C-2022- | Jun. 8, 2022 | JT-004 | Primary | Quantitative | 86.77 | >100.00 | >100.00 | >1 | 1 |
| 60269 | | | | polymerase chain | | | | | |
| | | | | reaction | | | | | |
| | | | | (DNA)/CellTiter-Glo | | | | | |
| | | | | (Toxicity) | | | | | |
| C-2022- | Jun. 8, 2022 | JT-006 | Primary | Quantitative | 79.91 | >100.00 | >100.00 | >1 | 1 |
| 60271 | | | | polymerase chain | | | | | |
| | | | | reaction | | | | | |
| | | | | (DNA)/CellTiter-Glo | | | | | |
| | | | | (Toxicity) | | | | | |

TABLE 2
In Vitro Antiviral Screening Results (Epstein Barr Virus)

| | |
|---|---|
| Virus screened: | Epstein-Barr virus |
| Virus strain: | Akata |
| Cell line: | Akata |
| Vehicle: | DMSO |
| Drug conc. Range: | 0.032-100 μM |
| Control conc. Range: | 0.032-100 μM |
| Experiment number: | 22-ebv-012 |

| Control | Control | | | | | | |
|---|---|---|---|---|---|---|---|
| drug | assay | | | | | | |
| name | order | Control assay name | EC₅₀ | EC₉₀ | CC₅₀ | SI₅₀ | SI₉₀ |
| Cidofovir | Primary | Quantitative polymerase chain | 5.94 | 73.51 | >100.00 | >18 | >1 |
| | | reaction (DNA)/CellTiter-Glo | | | | | |
| | | (Toxicity) | | | | | |

EC₅₀—compound concentration that reduces viral replication by 50%
SI₅₀—CC₅₀/EC₅₀
EC₉₀—compound concentration that reduces viral replication by 90%
SI₉₀—CC₅₀/EC₉₀
CC₅₀—compound concentration that reduces cell viability by 50%

Screening Results

| | Date | | | | | | | | |
|---|---|---|---|---|---|---|---|---|---|
| Product | Received | Compound | Drug | | | | | | |
| number | (m/dd/yy) | name/ID | assay order | Drug assay name | EC₅₀ | EC₉₀ | CC₅₀ | SI₅₀ | SI₉₀ |
| C-2022- | Jul. 6, 2022 | CHK-05 | Primary | Quantitative | 69.85 | >100.00 | >100.00 | >1 | >5 |
| 60241 | | | | polymerase chain | | | | | |
| | | | | reaction | | | | | |
| | | | | (DNA)/CellTiter-Glo | | | | | |
| | | | | (Toxicity) | | | | | |

TABLE 3
In Vitro Antiviral Screening Results (MERS Coronavirus)

| | |
|---|---|
| Virus screened: | MERS Coronavirus |
| Virus strain: | EMC |
| Cell line: | Vero 76 |
| Vehicle: | DMSO |
| Drug conc. Range: | 0.1-100 μM |
| Control conc. Range: | 0.1-100 μg/mL |
| Experiment number: | MERS-303 |
| Drug assay order: | Primary |

| Control | Control | | | | | | |
|---|---|---|---|---|---|---|---|
| drug | assay | | | | | | |
| name | order | Control assay name | EC₅₀ | EC₉₀ | CC₅₀ | SI₅₀ | SI₉₀ |
| EIDD- | Primary | Visual (Cytopathic effect/Toxicity) | 0.036 | | >10 | >280 | |
| 1931 | | | | | | | |
| EIDD- | Primary | Neutral Red (Cytopathic | 0.034 | | >10 | >290 | |
| 1931 | | effect/Toxicity) | | | | | |

EC₅₀—compound concentration that reduces viral replication by 50%
SI₅₀—CC50/EC50
EC₉₀—compound concentration that reduces viral replication by 90%
SO₉₀—CC50/EC90
CC₅₀—compound concentration that reduces cell viability by 50%
Compounds with SI values >10 are considered active and merit further investigation

Screening Results

| | | | | | | | | | |
|---|---|---|---|---|---|---|---|---|---|
| | Date | | | | | | | | |
| Product | Received | | Compound | | | | | | |
| number | (m/dd/yy) | ARB# | name/ID | Drug assay name | EC₅₀ | EC₉₀ | CC₅₀ | SI₅₀ | SI₉₀ |

| | | | | | | | |
|---|---|---|---|---|---|---|---|
| C-2022- | Jul. 6, 2022 | C- | CHK-05 | Visual (Cytopathic | >100 | 100 | 0 |
| 60241 | | 2022- | | effect/Toxicity) | | | |
| | | 60241 | | | | | |
| C-2022- | Jul. 6, 2022 | C- | CHK-05 | Neutral Red | >45 | 45 | 0 |
| 60241 | | 2022- | | (Cytopathic | | | |
| | | 60241 | | effect/Toxicity) | | | |

TABLE 4
In Vitro Antiviral Screening Results (Enterovirus-71)

| | |
|---|---|
| Virus screened: | Dengue Virus 2 |
| Virus strain: | New Guinea C |
| Cell line: | Huh7 |
| Vehicle: | DMSO |
| Drug conc. Range: | 0.1-100 μM |
| Control conc. Range: | 0.1-100 μM |
| Experiment number: | DV-467 |
| Drug assay order: | Primary |

| Control | Control | | | | | | |
|---|---|---|---|---|---|---|---|
| drug | assay | | | | | | |
| name | order | Control assay name | EC₅₀ | EC₉₀ | CC₅₀ | SI₅₀ | SI₉₀ |
| NITD008 | Primary | Visual (Cytopathic effect/Toxicity) | 0.36 | | >100 | >280 | |
| NITD008 | Primary | Neutral Red (Cytopathic | 0.43 | | >100 | >230 | |
| | | effect/Toxicity) | | | | | |

EC₅₀—compound concentration that reduces viral replication by 50%
SI₅₀—CC50/EC50
EC₉₀—compound concentration that reduces viral replication by 90%
SO₉₀—CC50/EC90
CC₅₀—compound concentration that reduces cell viability by 50%
Compounds with SI values >10 are considered active and merit further investigation

Screening Results

| | Date | | | | | | | | |
|---|---|---|---|---|---|---|---|---|---|
| Product | Received | | Compound | | | | | | |
| number | (m/dd/yy) | ARB# | name/ID | Drug assay name | EC₅₀ | EC₉₀ | CC₅₀ | SI₅₀ | SI₉₀ |
| C-2022- | Jul. 6, 2022 | C- | CHK-05 | Visual (Cytopathic | 79 | | >100 | >1.3 | |
| 60241 | | 2022- | | effect/Toxicity) | | | | | |
| | | 60241 | | | | | | | |

TABLE 5
In Vitro Antiviral Screening Results (Chikungunya Virus)

| | |
|---|---|
| Virus screened: | Chikungunya Virus |
| Virus strain: | S27, ATCC VR-64 |
| Cell line: | HVero 76 |
| Vehicle: | DMSO |
| Drug conc. Range: | 0.1-100 μM |
| Control conc. Range: | 0.1-100 μg/mL |
| Experiment number: | CHIKV-286 |
| Drug assay order: | Primary |

| Control | Control | | | | | | |
|---|---|---|---|---|---|---|---|
| drug | assay | | | | | | |
| name | order | Control assay name | EC₅₀ | EC₉₀ | CC₅₀ | SI₅₀ | SI₉₀ |
| EIDD- | Primary | Visual (Cytopathic effect/Toxicity) | 3.2 | | >100 | >31 | |
| 1931 | | | | | | | |
| EIDD- | Primary | Neutral Red (Cytopathic | 7.9 | | >100 | >13 | |
| 1931 | | effect/Toxicity) | | | | | |

EC₅₀—compound concentration that reduces viral replication by 50%
SI₅₀—CC50/EC50
EC₉₀—compound concentration that reduces viral replication by 90%
SO₉₀—CC50/EC90
CC₅₀—compound concentration that reduces cell viability by 50%
Compounds with SI values >10 are considered active and merit further investigation

Screening Results

| | Date | | | | | | | | |
|---|---|---|---|---|---|---|---|---|---|
| Product | Received | | Compound | | | | | | |
| number | (m/dd/yy) | ARB# | name/ID | Drug assay name | EC₅₀ | EC₉₀ | CC₅₀ | SI₅₀ | SI₉₀ |
| C-2022- | Jul. 6, 2022 | C- | CHK-05 | Visual (Cytopathic | >68 | | 68 | 0 | |
| 60241 | | 2022- | | effect/Toxicity) | | | | | |
| | | 60241 | | | | | | | |
| C-2022- | Jul. 6, 2022 | C- | CHK-05 | Neutral Red | >42 | | 42 | 0 | |
| 60241 | | 2022- | | (Cytopathic | | | | | |
| | | 60241 | | effect/Toxicity) | | | | | |

TABLE 6
In Vitro Antiviral Screening Results (Ebola Virus)

| Virus screened: | Ebola Virus | |
|---|---|---|
| Virus strain: | Zaire | |
| Cell line: | Vero | |
| Vehicle: | DMSO | |
| Drug conc. Range: | 0.1-100 | μM |
| Control conc. Range: | 7.8-1000 | μM |

| | |
|---|---|
| Experiment number: | EBOV-17Jun22 |
| Drug assay order: | Primary |

| | Control | | | | | | |
|---|---|---|---|---|---|---|---|
| Control | assay | | | | | | |
| drug name | order | Control assay name | EC₅₀ | EC₉₀ | CC₅₀ | SI₅₀ | SI₉₀ |
| Favipiravir | Primary | Visual (Cytopathic effect/Toxicity) | 12 | | >1000 | >83 | |
| Favipiravir | Primary | Neutral Red (Cytopathic | | | | | |
| | | effect/Toxicity) | | | | | |

EC₅₀—compound concentration that reduces viral replication by 50%
SI₅₀—CC50/EC50
EC₉₀—compound concentration that reduces viral replication by 90%
SO₉₀—CC50/EC90
CC₅₀—compound concentration that reduces cell viability by 50%
Compounds with SI values >10 are considered active and merit further investigation

Screening Results

| | Date | | | | | | | | |
|---|---|---|---|---|---|---|---|---|---|
| Product | Received | | Compound | | | | | | |
| number | (m/dd/yy) | ARB# | name/ID | Drug assay name | EC₅₀ | EC₉₀ | CC₅₀ | SI₅₀ | SI₉₀ |
| C-2022- | Jul. 6, 2022 | C- | CHK-05 | Crystal Violet | 18 | | >100 | >5.6 | |
| 60241 | | 2022- | | (Plaque | | | | | |
| | | 60241 | | Reduction)/Neutral | | | | | |
| | | | | Red (Toxicity) | | | | | |
| C-2022- | Jun. 9, 2022 | C- | JT-005 | Crystal Violet | 12 | | >100 | >8.3 | |
| 60270 | | 2022- | | (Plaque | | | | | |
| | | 60270 | | Reduction)/Neutral | | | | | |
| | | | | Red (Toxicity) | | | | | |

TABLE 7
In Vitro Antiviral Screening Results (Dengue Virus 2)

| | |
|---|---|
| Virus screened: | Dengue Virus 2 |
| Virus strain: | New Guinea C |
| Cell line: | Huh7 |
| Vehicle: | DMSO |
| Drug conc. Range: | 0.1-100 μM |
| Control conc. Range: | 0.1-100 μM |
| Experiment number: | DV 467 |
| Drug assay order: | Primary |

| Control | Control | | | | | | |
|---|---|---|---|---|---|---|---|
| drug | assay | | | | | | |
| name | order | Control assay name | EC₅₀ | EC₉₀ | CC₅₀ | SI₅₀ | SI₉₀ |
| NITD008 | Primary | Visual (Cytopathic effect/Toxicity) | 0.36 | | >100 | >280 | |
| NITD008 | Primary | Neutral Red (Cytopathic | 0.43 | | >100 | >230 | |
| | | effect/Toxicity) | | | | | |

EC₅₀—compound concentration that reduces viral replication by 50%
SI₅₀—CC50/EC50
EC₉₀—compound concentration that reduces viral replication by 90%
SO₉₀—CC50/EC90
CC₅₀—compound concentration that reduces cell viability by 50%
Compounds with SI values >10 are considered active and merit further investigation

Screening Results

| | Date | | | | | | | | |
|---|---|---|---|---|---|---|---|---|---|
| Product | Received | | Compound | | | | | | |
| number | (m/dd/yy) | ARB# | name/ID | Drug assay name | EC₅₀ | EC₉₀ | CC₅₀ | SI₅₀ | SI₉₀ |
| C-2022- | Jul. 6, 2022 | C- | CHK-05 | Visual (Cytopathic | 79 | | >100 | >1.3 | |
| 60241 | | 2022- | | effect/Toxicity) | | | | | |
| | | 60241 | | | | | | | |

TABLE 8
In Vitro Antiviral Screening Results (Rubella)

| | |
|---|---|
| Virus screened: | Rubella Virus |
| Virus strain: | |
| Cell line: | A549 |
| Vehicle: | DMSO |
| Drug conc. Range: | |
| Control conc. Range: | |
| RA number: | RA-0001522 |
| Drug assay order: | |

IC₅₀—compound concentration that reduces viral replication by 50%
IC₉₀—compound concentration that reduces viral replication by 90%
CC₅₀—compound concentration that reduces cell viability by 50%

Screening Results

| | | | | | | |
|---|---|---|---|---|---|---|
| | Date | | | | | |
| Product | Received | Compound | Drug assay | | | |
| number | (m/dd/yy) | name/ID | name | IC₅₀ | IC₉₀ | CC₅₀ |

| | | | |
|---|---|---|---|
| CDW3-002 | 69.89 | >100 | 97.15 |

TABLE 9
In Vitro Antiviral Screening Results (Rift Valley Fever Virus)

| | |
|---|---|
| Virus screened: | Rift Valley Fever Virus |
| Virus strain: | |
| Cell line: | A551 |
| Vehicle: | DMSO |
| Drug conc. Range: | |
| Control conc. Range: | |
| RA number: | RA-0001522 |
| Drug assay order: | |

IC₅₀—compound concentration that reduces viral replication by 50%
IC₉₀—compound concentration that reduces viral replication by 90%
CC₅₀—compound concentration that reduces cell viability by 50%

Screening Results

| | | | | | | |
|---|---|---|---|---|---|---|
| | Date | | | | | |
| Product | Received | Compound | Drug assay | | | |
| number | (m/dd/yy) | name/ID | name | IC₅₀ | IC₉₀ | CC₅₀ |

| | | | |
|---|---|---|---|
| CDW3-002 | 74.87 | >100 | >100 |

### Example 2

The following compounds were synthesized according to methods described herein.

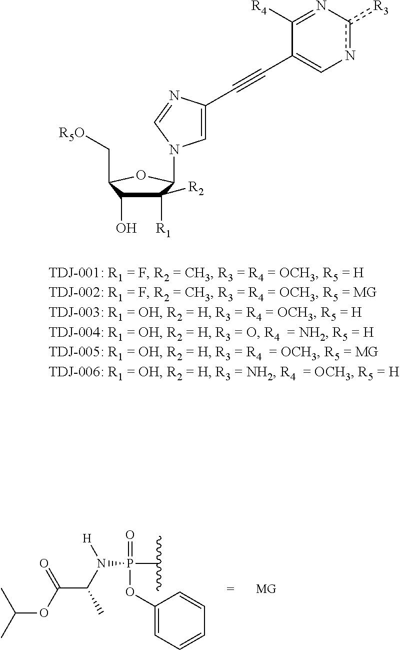

The antiviral activity of the compounds was tested as described herein and the results presented in Table 10 below:

TABLE 10
Ebola

| | |
|---|---|
| TDJ-001 | EC₅₀ > 100 μM |
| TDJ-002 | EC₅₀ = 44.2 μM (CC₅₀ > 200 μM) |
| TDJ-003 | IC₅₀ = 22.3 μM (CC₅₀ > 100 μM) |
| TDJ-004 | IC₅₀ = 20.9 μM (CC₅₀ > 100 μM) |
| TDJ-005 | IC₅₀ = 23.6 μM (CC₅₀ = 119 μM) |

SARS-CoV-2

| | |
|---|---|
| TDJ-002 | IC₅₀ = 34.20 μM |

Dengue

| | |
|---|---|
| TDJ-003 | IC₅₀ > 50 μM |
| TDJ-004 | IC₅₀ > 50 μM |

Chikungunya

| | |
|---|---|
| TDJ-002 | IC₅₀ = 54.29 μM |

### Example 3

The following compounds were synthesized according to methods described herein:

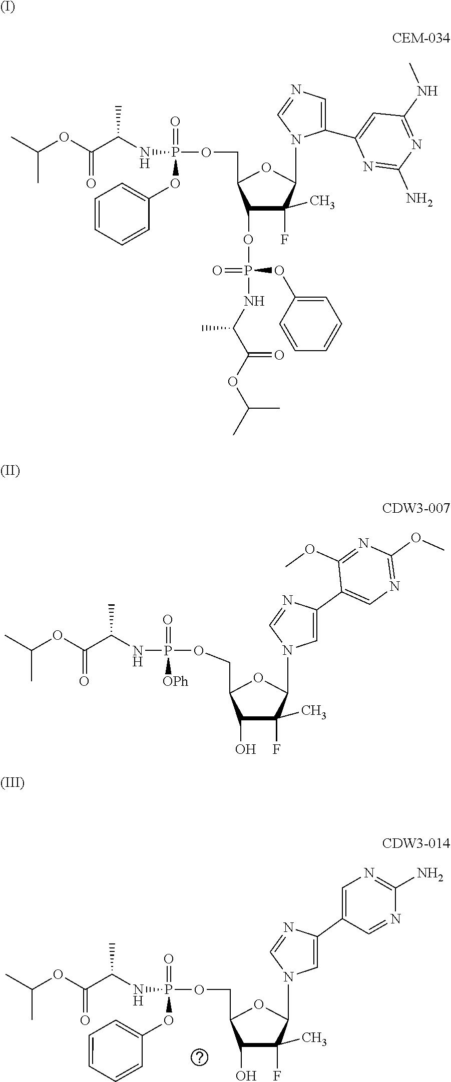

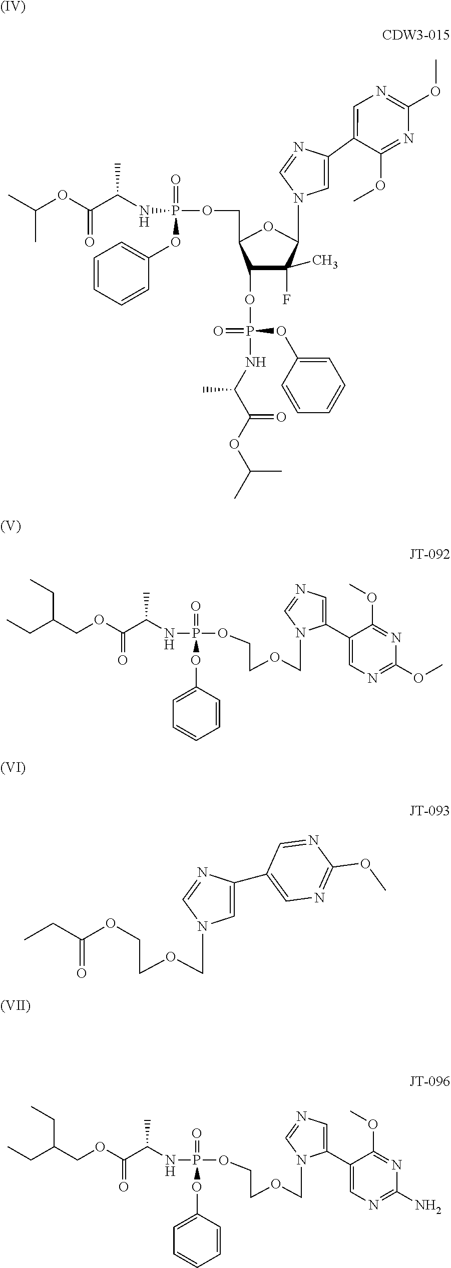

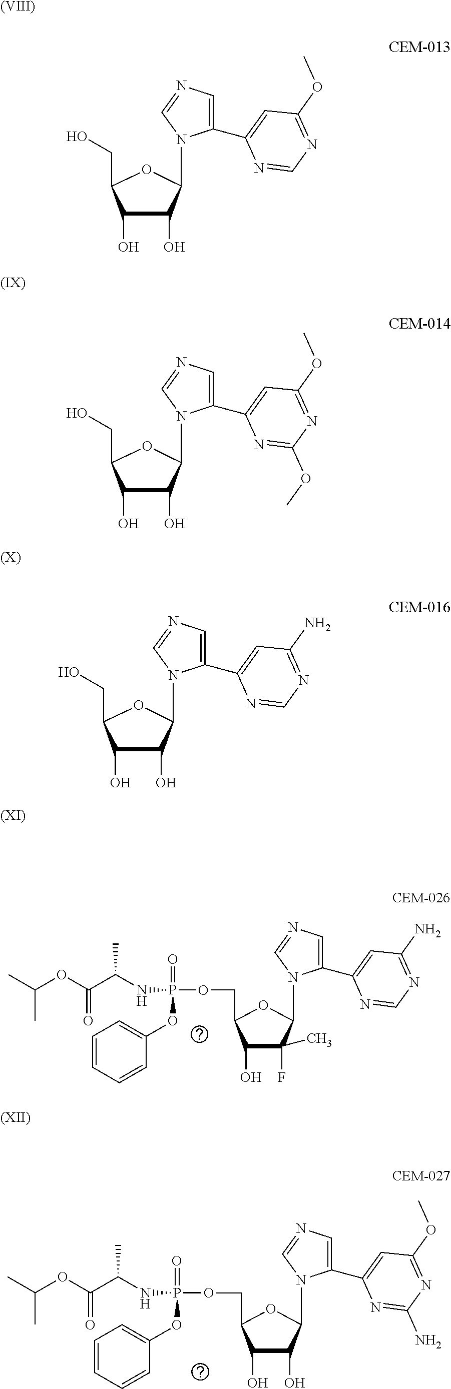

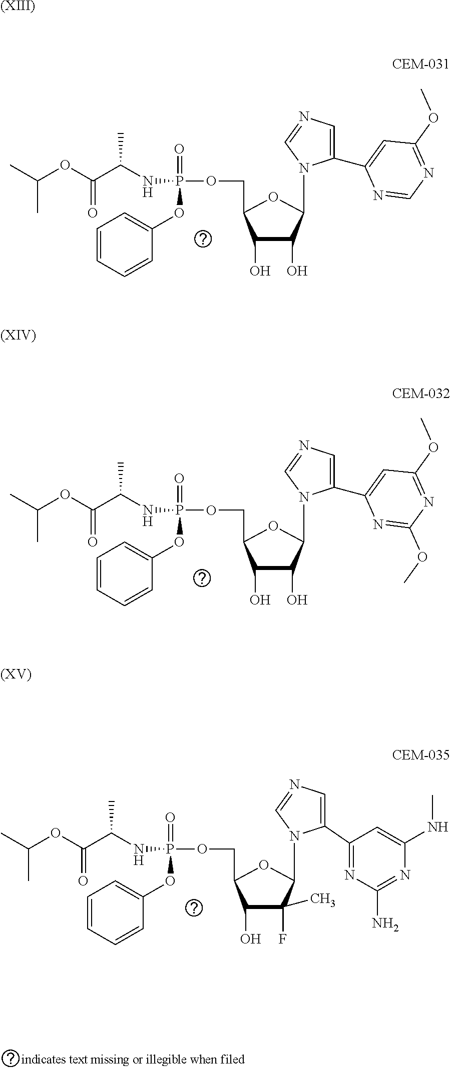

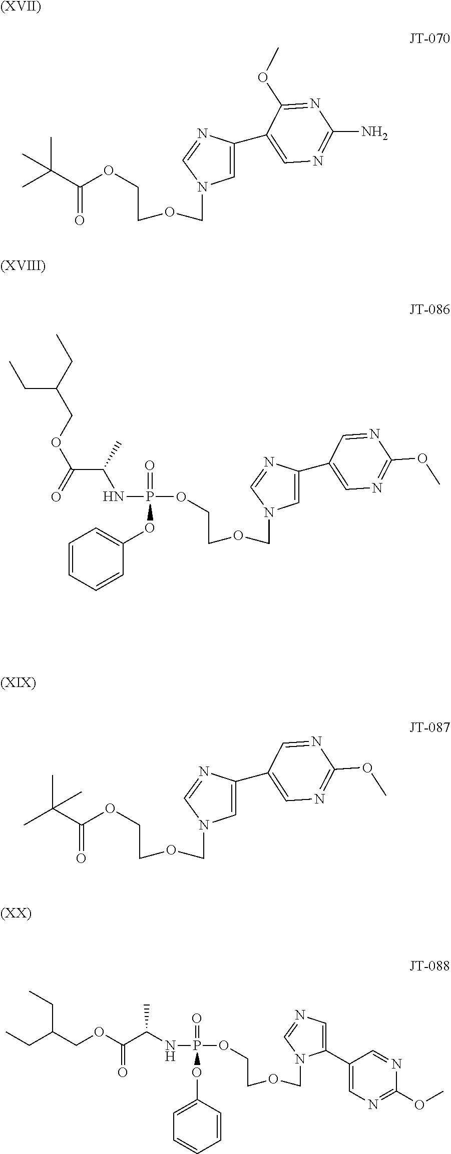

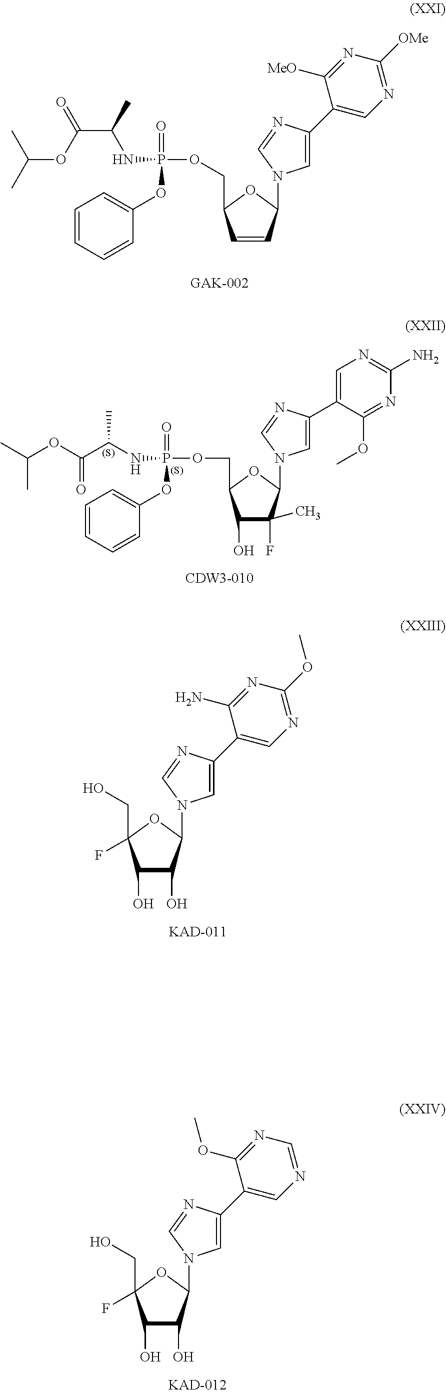

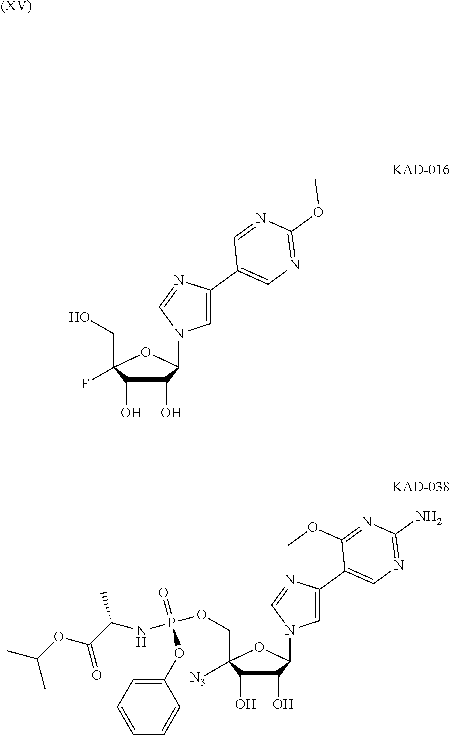

The results of the effectiveness of specific structures relating to different viruses are shown below in Tables 11-12. When reviewing the results, it should be noted that the lower the IC₅₀, the lower the concentration of drug required to produce 50% of maximum effect and thus, the higher the potency of the drug.

TABLE 11
IC₅₀ results.

| Virus | CEM-034 | CDW3-007 | CDW3-014 | CDW3-015 |
|---|---|---|---|---|
| DENV2 | IC50 = 14 μM | IC50 = 45 μM | 13.64% inhib | IC50 = 10 μM |
| | | | @50 μM | IC90 = 7.4 μM |
| DENV4 | IC50 = 18 μM | IC50 > 50 μM | 27.01% inhib | IC50 = 16 μM |
| | | | @50 μM | IC90 = 26 μM |
| | | | | CC50 > 100 μM |
| SARS-CoV-2 (Omicron) | IC50 = 22.6 μM | | | IC50 = 19.705 μM |
| (Calu3) #1 | CC50 = 35.41 μM | | | CC50 = 1.744 μM |
| SARS-CoV-2 | IC50 = 13.6 μM | | | IC50 = 12.387 μM |
| (WA)(A549) #1 | CC50 = 4.05 μM | | | CC50 > 100 μM |
| SARS-CoV-2 (A549) #2 | IC50 = 6.47 μM | | | IC50 = 2.947 μM |
| | CC50 = 15.44 μM | | | |
| EBOV (VP30, Huh) | IC50 = 13.5 μM | IC50 = 13 μM | IC50 = 10.5 μM | IC50 = 47.0 μM |
| | (Huh7) | CC50 = 274 μM | CC50 = 33.4 μM | CC50 > 200 μM |
| | CC50 = 85 μM | IC50 = 30.2 μM | | |
| | | CC50 > 200 μM | | |
| CHIKV | EC50 = 19.94 μM | | | |
| | CC50 > 100 μM | | | |
| VEEV | EC50 = 19.7 μM | | | |
| | CC50 > 100 μM | | | |

DENV2 = Dengue Virus 2; DENV4 = Dengue Virus 4; EBOV = Ebola Virus; CHIKV = Chikungunya Virus; VEEV = Venezuelan Equine Encephalitis Virus; OC43 = human coronavirus; SARS-CoV-2 (Omicron) was the Omicron strain in Calu-3 cells; SARS-CoV-2 (WA-1) was the WA-1 strain in A549 cells; #1 was a different lab than #2.

TABLE 12

| Virus | JT-092 | JT-093 | JT-096 |
|---|---|---|---|
| DENV2 | | IC50 = 21 μM | IC50 > 28 μM |
| DENV4 | | IC50 > 50 μM | IC50 > 42 μM |
| SARS-CoV-2 (Omicron) | | | |
| (Calu3) #1 | | | |
| SARS-CoV-2 | IC50 = 29.711 μM | | IC50 = 10.497 μM |
| (WA)(A549) #1 | IC90 = 272.98 μM | | IC90 = 46.317 μM |
| | CC50 > 100 μM | | CC50 = 73.259 μM |
| SARS-CoV-2 | IC50 = 82.777 μM | | IC50 = 31.414 μM |
| (WA)(Calu3) | IC90 = 172.18 μM | | IC90 = 106.1 μM |
| | CC50 > 100 μM | | CC50 > 100 μM |
| EBOV (VP30, Huh) | | | IC50 = 52 μM on |
| | | | Huh7 cells |
| | | | CC50 = 28 μM |

### Example 4

In some other embodiments, the flexible nucleoside analogue comprises at least one of:

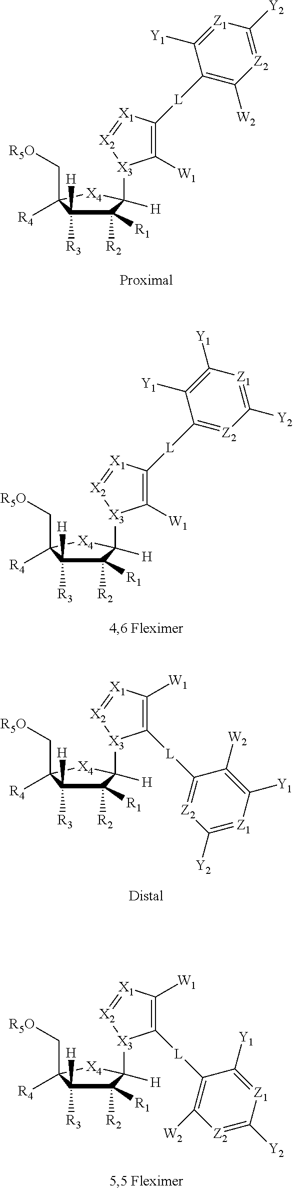

wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, CF₃, N-(alkyl)₂, NH-alkyl, O-alkyl, NH—OH, —NH—C(═O)(alkyl), or various prodrugs (e.g., esters, McGuigan ProTides diisoproxyl fumarate, lipid phosphates, lipid esters, HepDirect, amino acid prodrugs);
Z₁ and Z₂ are each independently selected from C or a heteroatom;
R₁ and R₂ are each independently selected from H, OH, F, Cl, CH₃, ethynyl, or —O—C(═O)(alkyl);
R₃ is H, OH, N₃, various prodrugs (e.g., esters, amino acid prodrugs), or —O—C(═O)(alkyl);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs), or —C(═O)(alkyl),
   or a pharmaceutically acceptable salt, isomer, hydrate, prodrug or solvate thereof.

The following compounds were synthesized according to methods described herein:

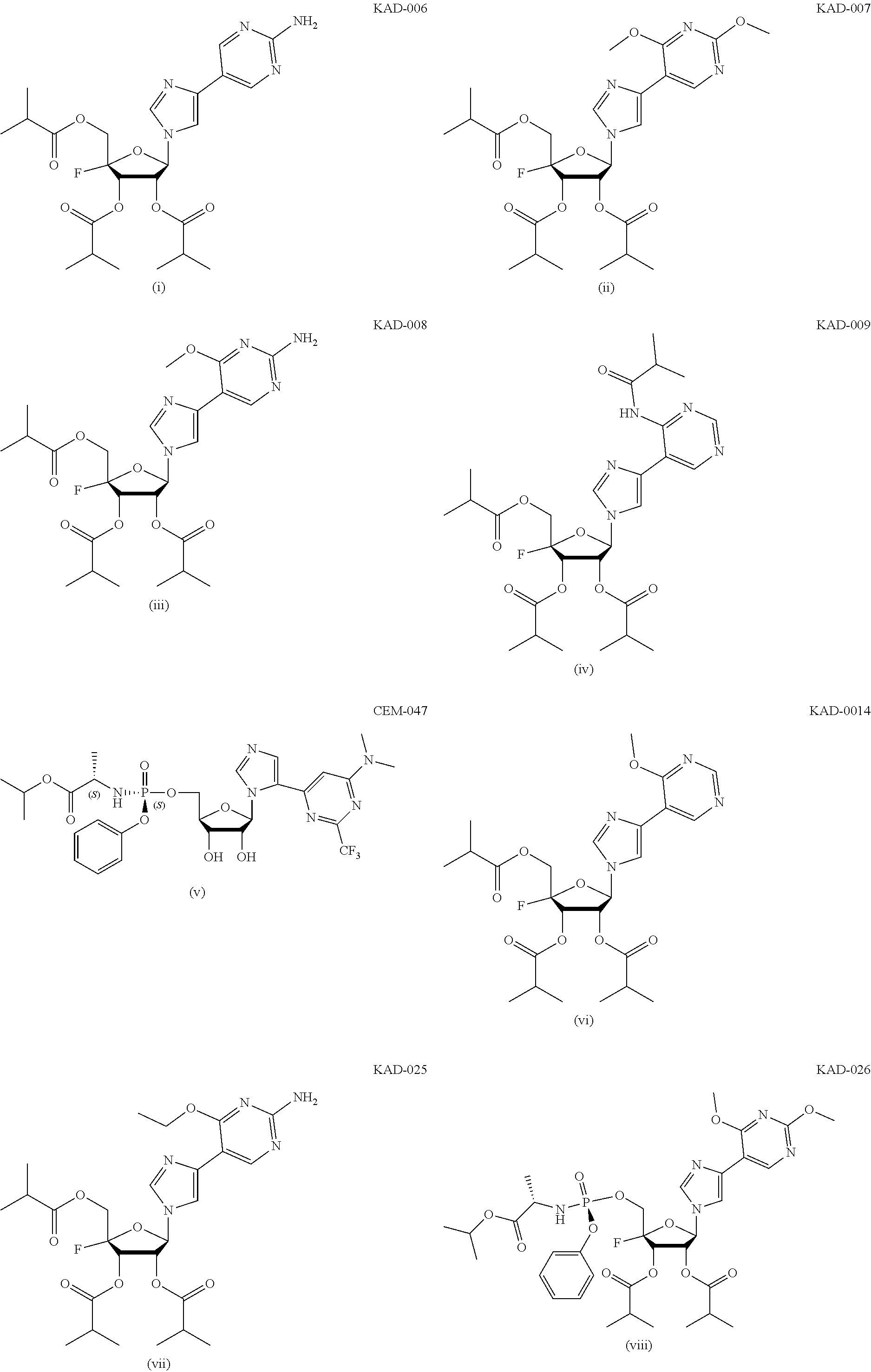

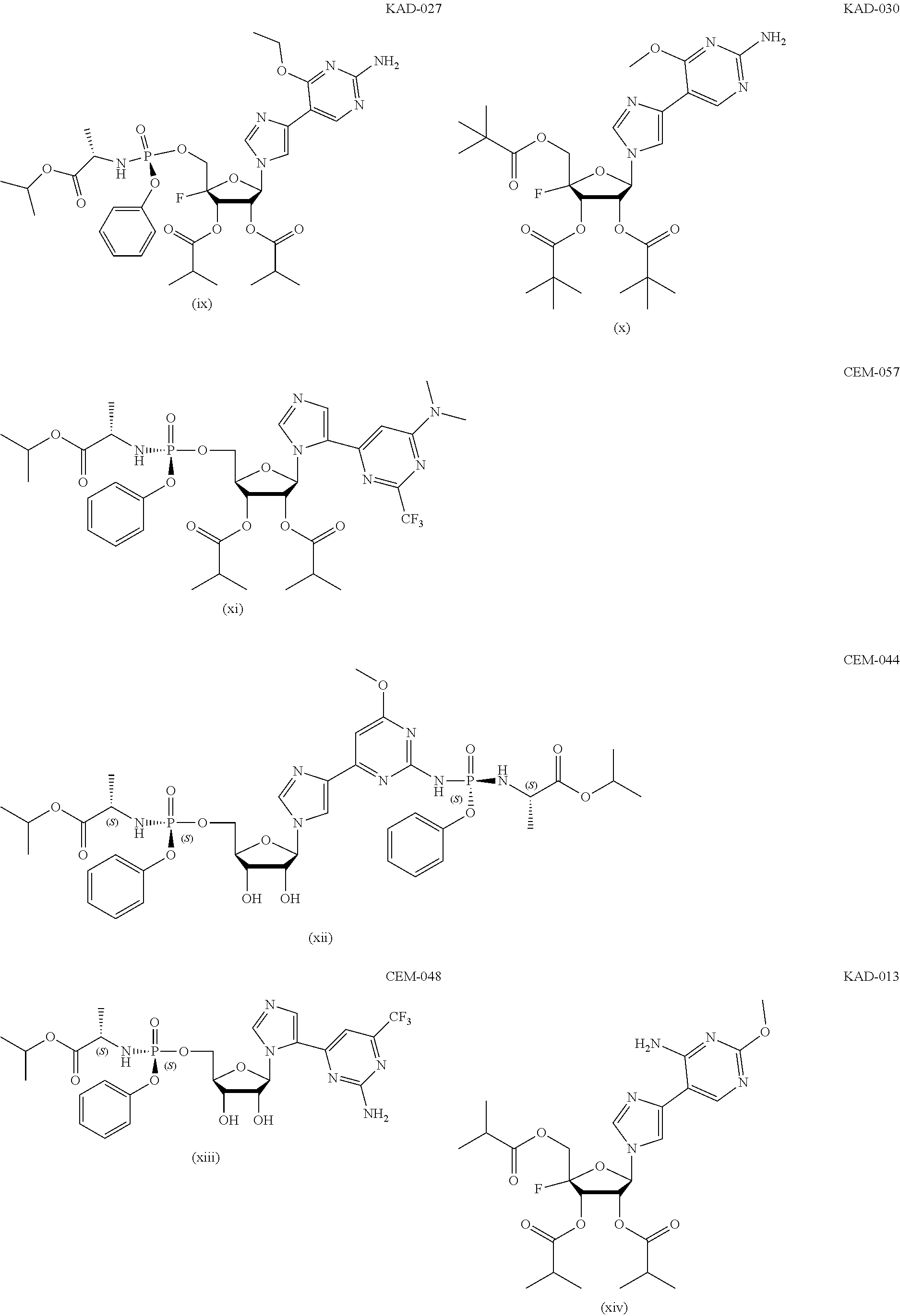

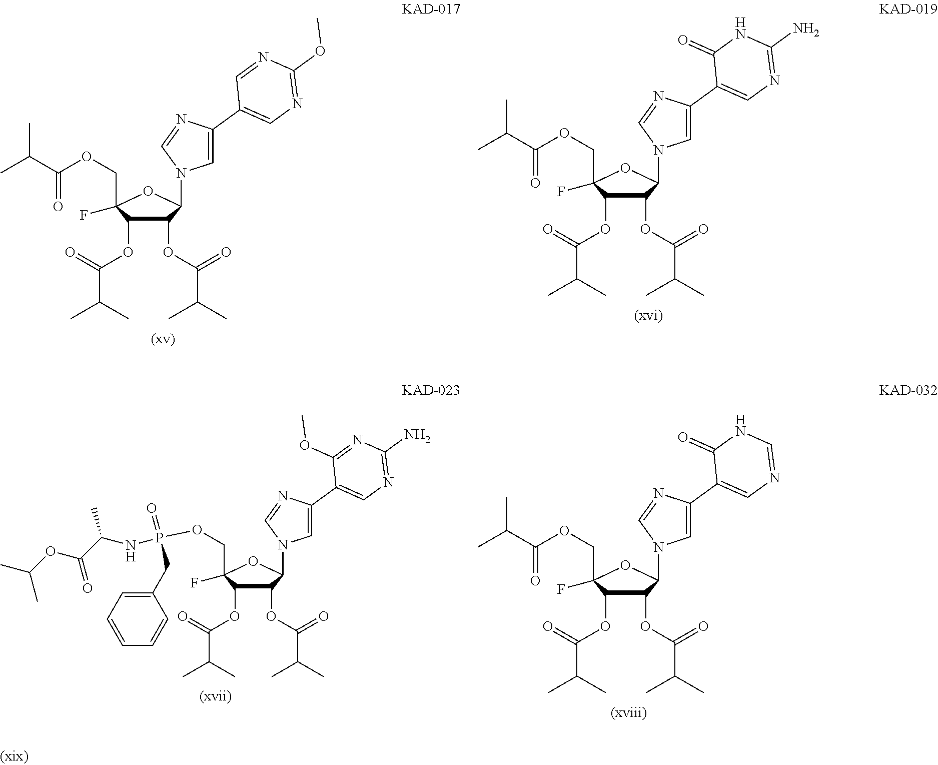

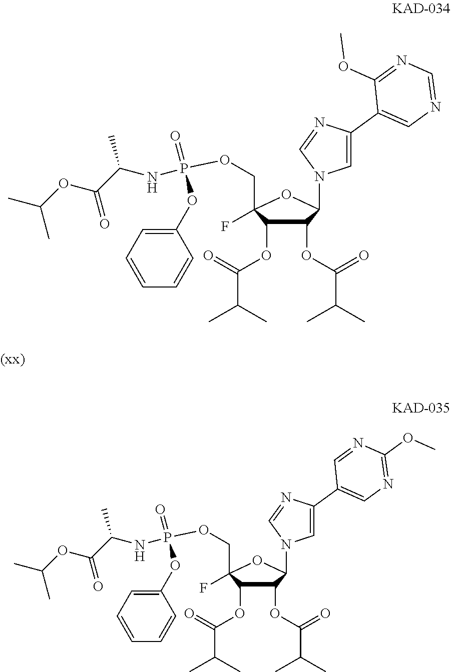

The results of the effectiveness of specific structures relating to different viruses are shown below in Tables 13-15.

TABLE 13

| Virus | KAD-006 | KAD-007 | KAD-008 | KAD-009 |
|---|---|---|---|---|
| DENV2 | IC50 = 27.0 μM | IC50 = 16.0 μM | IC50 = 2.3 μM | IC50 = 33.0 μM |
| | IC90 > 50 μM | IC90 = 27 μM | IC90 = 30 μM | |
| DENV4 | IC50 = 18 μM | IC50 = 23.0 μM | IC50 = 12.0 μM | IC50 = 49.0 μM |
| | IC90 > 50 μM | IC90 > 50 μM | IC90 > 50 μM | CC50 > 100 μM |
| | CC50 > 50 μM | CC50 > 100 μM | CC50 > 100 μM | |
| SARS-CoV-2 (Omicron) | IC50 > 100 μM | IC50 > 100 μM | IC50 = 60.825 μM | IC50 > 100 μM |
| (Calu3) #1 | CC50 = 77.587 μM | CC50 = 77.587 μM | CC50 = 41.272 μM | CC50 = 77.587 μM |
| SARS-CoV-2 | IC50 = 79.517 μM | IC50 = 29.460 μM | IC50 = 20.832 μM | IC50 = 83.439 μM |
| (WA)(A549) #1 | CC50 = 88.338 μM | CC50 = 30.014 μM | CC50 = 26.729 μM | CC50 > 100 μM |
| SARS-CoV-2 | | | IC50 = 33.18 μM | IC50 = 25.04 μM |
| | | | CC50 = 90.96 μM | CC50 > 100 μM |
| EBOV (VP30, Huh) | IC50 = 56.74 μM | | | |
| | CC50 > 200 μM | | | |
| CHIKV | 0.80% inhib at | 27.49% inhib at | 54.94% inhib at | |
| | 100 μM | 100 μM | 100 μM | |

TABLE 14

| Virus | CEM-047 | KAD-014 | KAD-025 | KAD-026 |
|---|---|---|---|---|
| DENV2 | IC50 = 27.0 μM | IC90 > 50 μM | IC50 = 14.1 μM | IC50 = 24.2 μM |
| DENV4 | IC50 = 22 μM | IC90 = 22 μM | IC50 = 27.0 μM | IC50 = 35.5 μM |
| | CC50 > 100 μM | | | |
| SARS-CoV-2 (Omicron) | IC50 = 46.165 μM | | | |
| (Calu3) #1 | CC50 > 100 μM | | | |
| SARS-CoV-2 | IC50 = 24.425 μM | | | |
| (WA)(A549) #1 | CC50 > 100 μM | | | |
| SARS-CoV-2 | IC50 = 23.56 μM | IC50 = 39.80 μM | IC50 = 13.94 μM | |
| | CC50 > 100 μM | CC50 = 121.4 μM | CC50 > 40 μM | |
| EBOV (VP30, Huh) | | IC50 = 21.3 μM | IC50 = 85.6 μM | |
| | | CC50 > 100 μM | CC50 > 100 μM | |
| CHIKV | 92.28% inhib at | 19.47% inhib at | 85.98% inhib at | 32.28% inhib at |
| | 100 μM | 100 μM | 100 μM | 100 μM |

TABLE 15

| Virus | KAD-027 | KAD-030 | CEM-057 |
|---|---|---|---|
| DENV2 | IC50 = 26.3 μM | IC90 = 6.9 μM | IC90 = 6.5 μM |
| DENV4 | IC50 = 34.7 μM | IC90 = 6.3 μM | IC90 = 7.2 μM |
| SARS-CoV-2 (Omicron) | | | |
| (Calu3) #1 | | | |
| SARS-CoV-2 | | | |
| (WA)(A549) #1 | | | |
| SARS-CoV-2 | EC50 > 40 μM | | IC50 = 11.2 μM |
| | CC50 > 40 μM | | CC50 > 50 μM |
| EBOV (VP30, Huh) | IC50 = 68.5 μM | IC50 = 25.56 μM | IC50 = 5.45 μM |
| | CC50 > 100 μM | CC50 = 84 μM | CC50 = 41 μM |
| CHIKV | 77.48% inhib at | | |
| | 100 μM | | |

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Beadle J R, Valiaeva N, Yang G, Yu J H, Broker T R, Aldern K A, et al. Synthesis and Antiviral Evaluation of Octadecyloxyethyl Benzyl 9-[(2-Phosphonomethoxy)ethyl]guanine (ODE-Bn-PMEG), a Potent Inhibitor of Transient HPV DNA Amplification. J Med Chem. 2016; 59(23):10470-8. PubMed PMID: 27933957.
Hartline C B, Keith K A, Eagar J, Harden E A, Bowlin T L, Prichard M N. Antiviral Res. 2018. A standardized approach to the evaluation of antivirals against DNA viruses: Orthopox-, adeno-, and herpesviruses. November; 159:104-112. doi: 10.1016/j.antiviral.2018.09.015. Epub 2018 Oct. 1.PMID: 30287226
Keith K A, Hartline C B, Bowlin T L, Prichard M N. Antiviral Res. 2018. A standardized approach to the evaluation of antivirals against DNA viruses: Polyomaviruses and lymphotropic herpesviruses. November; 159:122-129. doi: 10.1016/j.antiviral.2018.09.016. Epub 2018 Oct. 1. PMID: 30287227
Leung A Y, Suen C K, Lie A K, Liang R H, Yuen K Y, Kwong Y L. Quantification of polyoma B K viruria in hemorrhagic cystitis complicating bone marrow transplantation. Blood. 2001; 98(6):1971-8. PubMed PMID: 11535537.
Prichard M N, Frederick S L, Daily S, Borysko K Z, Townsend L B, Drach J C, et al. Benzimidazole analogs inhibit human herpesvirus 6. Antimicrob Agents Chemother. 2011; 55(5):2442-5. PubMed PMID: 21300829; PubMed Central PMCID: PMC3088228.

That which is claimed is:

1. A flexible nucleoside analogue comprising at least one D-nucleoside fleximer selected from: 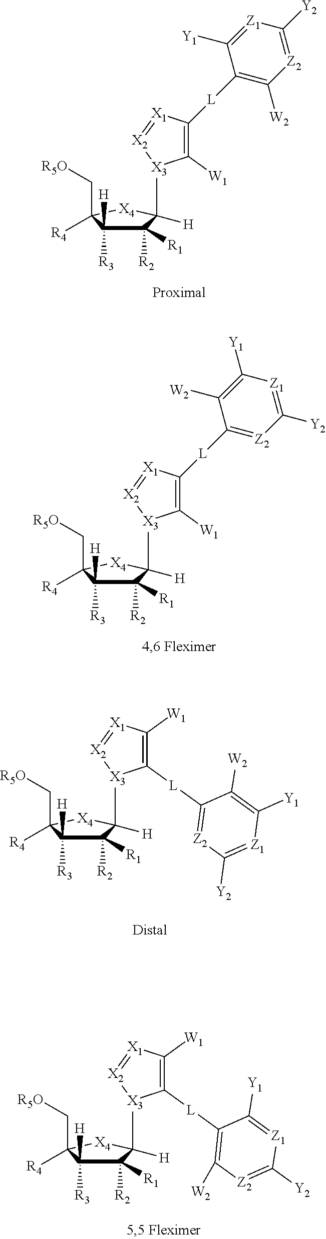
wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs),
or a pharmaceutically acceptable salt, isomer, hydrate, prodrug or solvate thereof.

2. The flexible nucleoside analogue of claim 1, comprising at least one compound selected from: 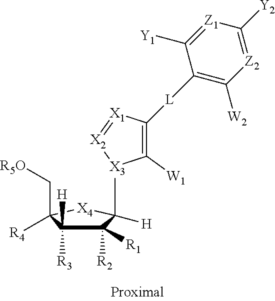
wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
X₄ is O, NH or S;
W₁ and W₂ are each independently selected from H, F, CH₃, CF₃, NH₂, OH, O-alkyl, or NH-alkyl;
Y₁ and Y₂ are each independently selected from H, ═O, OH, S, F, NH₂, NH-alkyl, O-alkyl, or NH—OH;
Z₁ and Z₂ are each independently selected from C or a heteroatom;
R₁ and R₂ are each independently selected from H, OH, F, Cl, CH₃ or ethynyl;
R₃ is H, OH, N₃, or various prodrugs (e.g., esters, amino acid prodrugs);
R₄ is H, F, N₃, ethynyl or cyano; and
R₅ is H, monophosphate, diphosphate, triphosphate, various prodrugs (e.g., esters, McGuigan ProTides, lipid phosphates, lipid esters, HepDirect, amino acid prodrugs),
or a pharmaceutically acceptable salt, isomer, hydrate, prodrug or solvate thereof.

3. The flexible nucleoside analogue of claim 1, comprising at least one of:
(i) X₁=X₃=N and X₂ is C;
(ii) Z₁=Z₂=N;
(iii) W₁=W₂=H;
(iv) R₁ is CH₃, R₂ is F, R₃ is OH and/or R₄ is H;
(v) L is null; or
(vi) any combination of (i)-(v).

4. The flexible nucleoside analogue of claim 2, wherein: X₁ and X₃ are each N; X₂ is C; X₄ is O; W₁ and W₂ are each H; Y₁ and Y₂ are each independently selected from H, ═O, OH, S, F, NH₂, NH-alkyl, O-alkyl, alkyl, or NH—OH; Z₁ and Z₂ are N; R₁ is alkyl; R₂ is F; R₃ is OH; R₄ is H; and R₅ is H, monophosphate, diphosphate, triphosphate, various prodrugs (e.g., esters, McGuigan ProTides, lipid phosphates, lipid esters, HepDirect, amino acid prodrugs),
or a pharmaceutically acceptable salt, isomer, hydrate, prodrug or solvate thereof.

5. The flexible nucleoside analogue of claim 1, wherein R₅ is

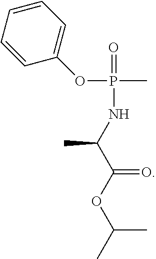 [SMILES: CC(C)OC(=O)[C@@H](C)NP(C)(=O)Oc1ccccc1]

6. The flexible nucleoside analogue of claim 1, wherein Y₂ is NH₂ and Y₁ is NH(CH₃).

7. The flexible nucleoside analogue of claim 1, wherein R₅ is H.

8. The flexible nucleoside analogue of claim 2, wherein when R₁ is CH₃, R₂ is F, R₃ is OH, R₄ is H, R₅ is a McGuigan Protide, W₁=W₂=H, Z₁=Z₂=N, L is null, X₁=X₃=N, X₂ is C, X₄ is O, and Y₂ is NH₂, Y₁ cannot be NH(CH₃).

9. The flexible nucleoside analogue of claim 1, comprising at least one of: 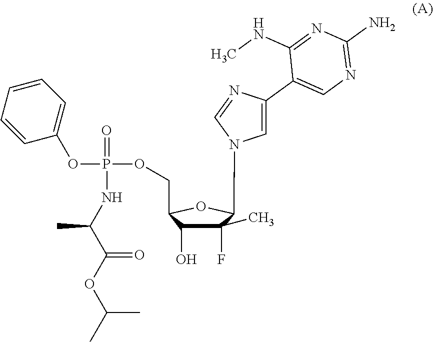
propan-2-yl-(2S)-2-(((((2R,3R,4R,5R)-5-(4-(2-amino-4-(methylamino)pyrimidin-5-yl)-1H-imidazol-1-yl)-4-fluoro-3-hydroxy-4-methyloxolan-2-yl)methyl) (phenyl)phosphono) amino) propanoate; 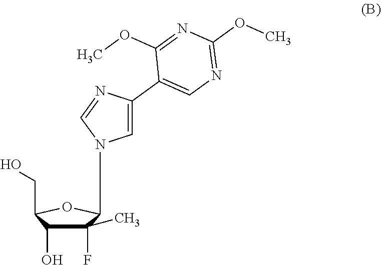
(2R,3R,4R,5R)-5-(4-(2,4-dimethoxypyrimidin-5-yl)-1H-imidazol-1-yl)-4-fluoro-2-(hydroxymethyl)-4-methyltetrahydrofuran-3-ol; 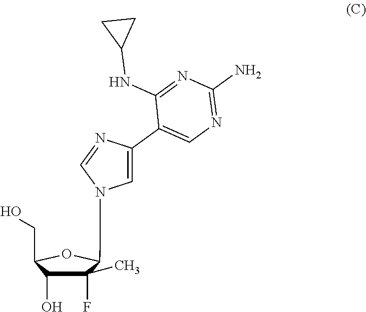
(2R,3R,4R,5R)-5-(4-(2-amino-4-(cyclopropylamino)pyrimidin-5-yl)-1H-imidazol-1-yl)-4-fluoro-2-(hydroxymethyl)-4-methyltetrahydrofuran-3-ol; 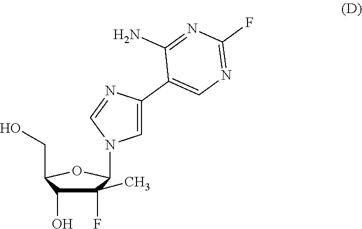
(2R,3R,4R,5R)-5-(4-(4-amino-2-fluoropyrimidin-5-yl)-1H-imidazol-1-yl)-4-fluoro-2-(hydroxymethyl)-4-methyltetrahydrofuran-3-ol; 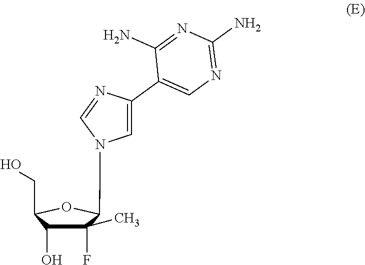
(2R,3R,4R,5R)-5-(4-(2,4-diaminopyrimidin-5-yl)-1H-imidazol-1-yl)-4-fluoro-2-(hydroxymethyl)-4-methyltetrahydrofuran-3-ol; 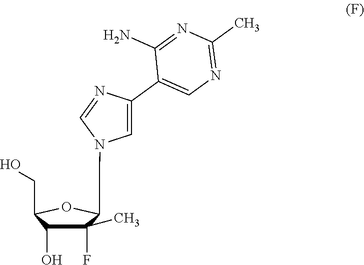
(2R,3R,4R,5R)-5-(4-(4-amino-2-methylpyrimidin-5-yl)-1H-imidazol-1-yl)-4-fluoro-2-(hydroxymethyl)-4-methyltetrahydrofuran-3-ol; 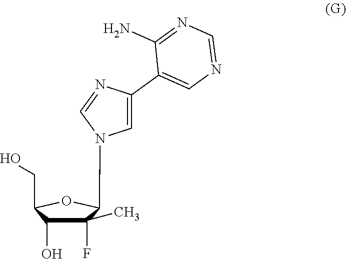
(2R,3R,4R,5R)-5-(4-(4-aminopyrimidin-5-yl)-1H-imidazol-1-yl)-4-fluoro-2-(hydroxymethyl)-4-methyltetrahydrofuran-3-ol; 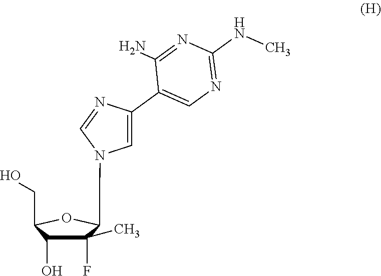
(2R,3R,4R,5R)-5-(4-(4-amino-2-(methylamino)pyrimidin-5-yl)-1H-imidazol-1-yl)-4-fluoro-2-(hydroxymethyl)-4-methyltetrahydrofuran-3-ol; 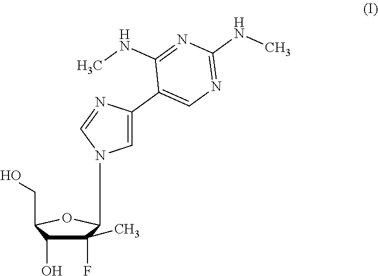
(2R,3R,4R,5R)-5-(4 (2,4-bis(methylamino)pyrimidin-5-yl)-1H-imidazol-1-yl)-4-fluoro-2-(hydroxymethyl)-4-methyltetrahydrofuran-3-ol; 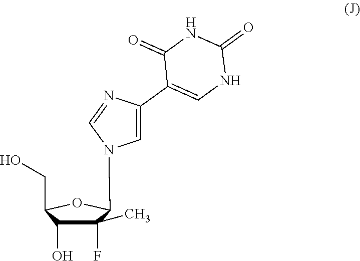
5-(1-((2R,3R,4R,5R)-3-fluoro-4-hydroxy-5-(hydroxymethyl)-3-methyltetrahydrofuran-2-yl)-1H-imidazol-4-yl)pyrimidine-2,4 (1H,3H)-dione; 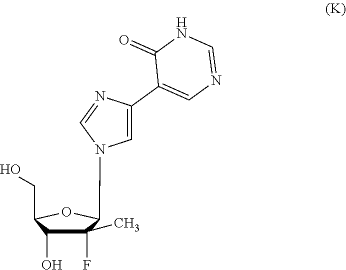
5-(1-((2R,3R,4R,5R)-3-fluoro-4-hydroxy-5-(hydroxymethyl)-3-methyltetrahydrofuran-2-yl)-1H-imidazol-4-yl)pyrimidin-4 (3H)-one; 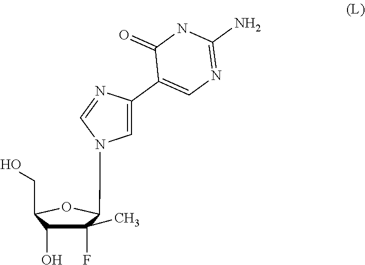
2-amino-5-(1-((2R,3R,4R,5R)-3-fluoro-4-hydroxy-5-(hydroxymethyl)-3-methyltetrahydrofuran-2-yl)-1H-imidazol-4-yl)pyrimidin-4 (3H)-one; and 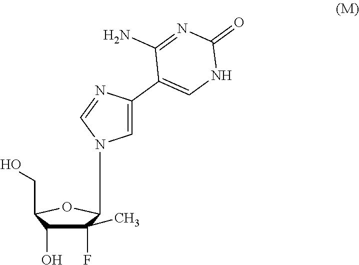
4-amino-5-(1-((2R,3R,4R,5R)-3-fluoro-4-hydroxy-5-(hydroxymethyl)-3-methyltetrahydrofuran-2-yl)-1H-imidazol-4-yl)pyrimidin-2 (1H)-one,
or a pharmaceutically acceptable salt, isomer, hydrate, prodrug or solvate thereof.

10. A pharmaceutical composition comprising at least one of the flexible nucleoside analogues of claim 1, and at least one pharmaceutically acceptable carrier.

11. The pharmaceutical composition of claim 10, further comprising at least one additional pharmaceutically active agent.

12. The pharmaceutical composition of claim 10, formulated as a syrup, elixir, tablet, troche, lozenge, hard or soft capsule, pill, suppository, oily or aqueous suspension, dispersible powder or granule, emulsion, injectable, solution, sustained release formulation, or aerosol.

13. A method for treating and/or preventing a viral infection in a subject, wherein the viral infection is caused by at least one of a coronavirus, a herpesvirus, an alphavirus, a polyomavirus, an enterovirus, a filovirus, a matonavirus, a phenuivirus, and/or a flavivirus, comprising administration, to the subject, of a therapeutically effective amount of at least one fleximer nucleoside analogue of claim 1.

14. The method of claim 13, wherein a therapeutically effective amount of the fleximer nucleoside analogue is from 0.05 to 50 mg per kilogram body weight of the subject per day.

15. The method of claim 13, wherein the method of administration is selected from the group consisting of systemically, orally, buccally, sublingually, topically, by inhalation, by spraying, intravenously, intramuscularly, subcutaneously, intrathecally, intradermally, intravascularly or intra-arterially.

16. The method of claim 13, wherein the viral infection is caused by:
a coronavirus selected from human coronaviruses (HCoV), Severe Acute Respiratory Syndrome Coronavirus (SARS-CoV), SARS-CoV-2, and Middle East respiratory syndrome (MERS), and mutants thereof;
a herpesvirus selected from herpes simplex virus 1, herpes simplex virus 2, varicella-zoster virus, Epstein-Barr virus, cytomegalovirus, Human herpesvirus-6, Human herpesvirus-7, and Kaposi's sarcoma herpes virus;
an alphavirus selected from eastern equine encephalomyelitis (EEE), Venezuelan equine encephalomyelitis (VEE), and western equine encephalomyelitis (WEE);
a polyomavirus;
an enterovirus selected from echovirus and coxsackievirus;
a filovirus selected from Zaire Ebola virus, Sudan Ebola virus, Reston Ebola virus, Cote d′Ivoire Ebola virus and Marburg virus;
a matonavirus selected from Rubella, Rustrela, and Ruhugu;
a phenuivirus selected from Rift Valley Fever virus; and/or
a flavivirus selected from the group consisting of yellow fever virus, Apoi virus, Aroa virus, Bagaza virus, Banzi virus, Bouboui virus, Bukalasa bat virus, Cacipacore virus, Carey Island virus, Cowbone Ridge virus, Dakar bat virus, dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Phenh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, tick-borne encephalitis virus, Tyuleniy virus, Uganda S virus, Usutu virus, Wesselsbron virus, West Nile virus, Yaounde virus, Yokose virus, Zika virus, cell fusing agent virus and Tamana bat virus.

17. A flexible nucleoside analogue selected from at least one of:
(i) at least one compound comprising a Flex-Acyclovir scaffold selected from: 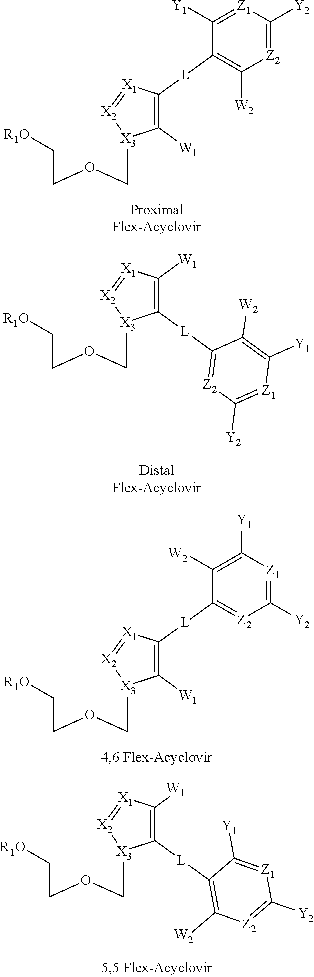
wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, monophosphate, diphosphate, triphosphate, various prodrugs (e.g., esters, McGuigan ProTides, lipid phosphates, lipid esters, HepDirect, amino acid prodrugs) or —C(O)-alkyl; and
(ii) at least one compound comprising a Flex-tenofovir scaffold selected from: 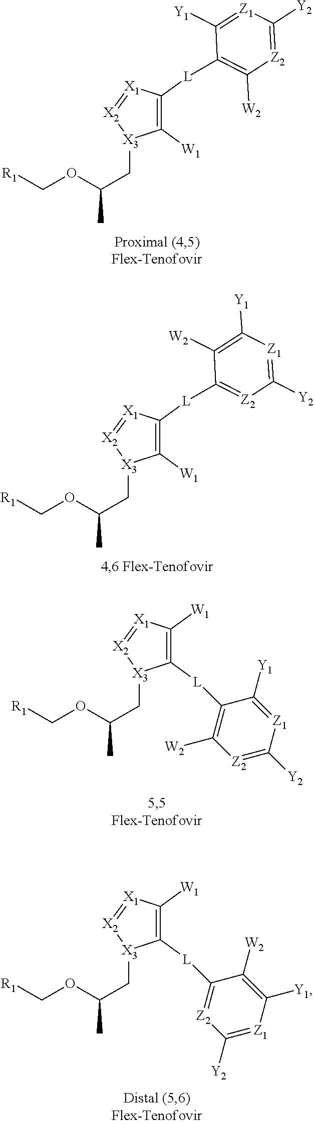
wherein
L is null, CH₂, S, vinyl, ethynyl, —O—(CH₂)ₙ—, or —NH—(CH₂)ₙ—;
n is any integer from 0-2;
X₁, X₂ and X₃ are each independently selected from C, O, N, or S;
Y₁ and Y₂ are each independently selected from H, OH, S, F, NH₂, NH-alkyl, O-alkyl, or NH—OH;
W₁ and W₂ are each independently selected from H, F, CH₃, CF₃, NH₂, OH, O-alkyl, or NH-alkyl;
Z₁ and Z₂ are each independently selected from C or a heteroatom; and
R₁ is H, phosphonate, monophosphate phosphonate, and diphosphate phosphonate, various prodrugs (e.g., esters, McGuigan ProTides, diisoproxyl fumarate, lipid phosphates, lipid esters, HepDirect, amino acid prodrugs),
or a pharmaceutically acceptable salt, isomer, hydrate, prodrug or solvate thereof.

18. A flexible nucleoside analogue having the structure: 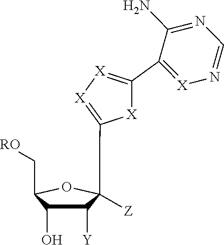
wherein
X is N or C;
Y is OH or F;
Z is H, CN, N₃ or C≡CH; and
R is H, acetoxy (OAc), monophosphate, diphosphate, triphosphate, various prodrugs such as McGuigan ProTides, Lipid phosphates, or esters,
or a pharmaceutically acceptable salt, isomer, hydrate, prodrug or solvate thereof.
